(19) United States
(12) Patent Application Publication
(10) Pub. No.: US 2024/0335149 A1
(43) Pub. Date: Oct. 10, 2024

(54) APPARATUSES AND METHODS FOR INTRAORAL PRESSURE SENSING

(71) Applicant: Align Technology, Inc., San Jose, CA (US)

(72) Inventors: Byong-Ho PARK, San Jose, CA (US); Sheyda NAZARIAN, San Jose, CA (US); Sangshik PARK, San Jose, CA (US); Zijie ZHU, San Jose, CA (US); Fred TING, San Jose, CA (US); Yixin XU, San Jose, CA (US); Vincent YEH, Fremont, CA (US); Hunho JO, San Jose, CA (US); Wesly WONG, Cupertino, CA (US); Zhichao ZHANG, Walnut Creek, CA (US); Robert NETTLES-MILLER, San Jose, CA (US)

(21) Appl. No.: 18/624,107

(22) Filed: Apr. 1, 2024

Related U.S. Application Data
(60) Provisional application No. 63/493,717, filed on Mar. 31, 2023.

(51) Int. Cl. A61B 5/22; A61B 5/00; A61B 5/08

(52) U.S. Cl. CPC A61B 5/228; A61B 5/0816; A61B 5/0826; A61B 5/4557; A61B 5/682; A61B 2562/0219; A61B 2562/0271

(57) ABSTRACT

Pressure sensor modules adapted for use with a dental appliance may include one or more pressure sensors encapsulated within a sealed cavity in which one or more walls of the sealed cavity are deformable so as to transduce pressure from outside of the cavity, due to deformation or movement of the dental appliance and/or the environment around the dental appliance, including the environment within a patient's mouth when wearing the dental appliance. Also described herein are method of making and using these apparatuses to identify and/or monitor the dental appliance, a patient condition and/or an orthodontic treatment. Also described herein are methods and apparatuses for identifying and/or characterizing sleep apnea using these pressure sensor modules.

### CLAIM OF PRIORITY

This patent application claims priority to U.S. Provisional Patent Application No. 63/493,717, titled “APPARATUSES AND METHODS FOR INTRAORAL PRESSURE SENSING,” and filed on Mar. 31, 2023, which is herein incorporated by reference in its entirety.

### BACKGROUND

Many physiological actions that occur in the vicinity of the oral cavity result in a change in intraoral pressure. Actions like breathing, swallowing, speaking, and snoring have distinct pressure profiles that can be measured and monitored for health sensing purposes. This invention details a specific configuration of intraoral pressure sensor, which can be placed in the intraoral environment as a fully scaled/encapsulated device. Deformability of the encapsulation materials allows the pressure sensor to transduce air pressure changes in the intraoral environment. Additionally, some clear advantages of this configuration when compared to previously disclosed atmospherically vented pressure sensors is the case of implementation (fewer materials and processes), inherent water resistance, and ability to perform mechanical force measurement.

### SUMMARY OF THE DISCLOSURE

Described herein are methods and apparatuses for sensing pressure within the oral cavity, e.g., oral pressure, as well as methods and apparatuses for using oral pressure, which may correspond to oral breathing, to detect, monitor and/or characterize (e.g., classify) one or more physiological states, including monitoring sleeping and/or sleep apnea. In general any of these methods and apparatuses may include a pressure sensor module that is adapted for use with a dental appliance, such as an orthodontic aligner, palatal expander, etc., and systems and methods including these pressure sensor modules.

The sealed pressure sensor modules described herein may include one or more pressure sensors encapsulated within a sealed cavity in which one or more walls of the sealed cavity are deformable so as to transduce pressure from outside of the cavity, e.g., due to deformation or movement of the dental appliance and/or the environment around the dental appliance, including the environment within a patient's mouth when wearing the dental appliance. Any of these apparatuses (e.g., devices, systems, etc.) may be configured to identify a condition or occurrence relevant to orthodontic treatment and/or the state of the dental appliance based on the sensed pressure.

For example, described herein are dental appliances comprising: a body forming one or more tooth-receiving cavities configured to be worn on a subject's teeth; a sealed pressure sensor module coupled to the body, the sealed pressure sensor module including: an airtight encapsulated cavity, wherein the airtight encapsulated cavity is encapsulated by one or more fluid-resistant (e.g., in some examples, fluid-impermeable) materials, wherein at least a portion of the one or more fluid-resistant materials forms a deformable surface; a pressure sensor encapsulated within the airtight encapsulated cavity and configured to measure pressure within the airtight encapsulated cavity, wherein the sealed pressure sensor module is held within a subject's mouth when the one or more tooth-receiving cavities are worn on the subject's teeth.

Any of these apparatuses may include a control circuitry receiving pressure data from the pressure sensor and configured to modify, store and/or transmit the pressure data. The control circuitry may include one or more processors, a memory, a controller, a communications module for communicating wirelessly (e.g., via BLUETOOTH, WI-FI, RF, ZIGBEE, etc.). Thus, the apparatus may include a wireless communications module as part of the pressure sensor module. The control circuitry may include a printed circuit board, chip, etc. The control circuitry may include a power control or power modulation module configured to control power to the components of the pressure sensor module, power supply, etc. The control circuitry may include a timer or clock. The control circuitry may include filters and/or amplifiers, e.g., for modulating via an analog and/or digital components, signals from the pressure sensor(s) and/or other sensors. The control circuity may be contained within the airtight encapsulated cavity, or it may be separate from the airtight encapsulated cavity and in electrical commination with the one or more sensors.

In general the control circuity may be configured to receive signals from the one or more pressure sensors and/or other sensors and may process, store and/or transmit signals from the one or more pressure sensors. In some examples the control circuity may include logic (e.g., analysis logic) for analyzing the signals (data signals) from the one or more pressure sensors and/or other sensors. In some examples the control circuity may include analysis logic that determines if the pressure signals, alone or in combination with one or more other sensor signals (e.g., temperature, accelerometer, etc.), indicate an external condition, a patient condition and/or a condition of the dental appliance. For example, the control circuitry may be configured so that the sealed pressure sensor module senses respiration and bruxism when the dental appliance is held within the subject's mouth.

Any of these apparatuses may include a power source. For example, the dental appliance may include a power source configured to apply power to the pressure sensor and to the control circuity. The power source may be a battery, capacitor, inductive coil, etc., or any combination of these. In some examples the power source is rechargeable. In some examples the power source is replaceable. The power source may be adapted for removing and/or recharging may be included in a region (e.g., cavity) that is separate from the airtight encapsulated cavity.

Any of the apparatuses described herein may include a palatal region extending between a first tooth-receiving cavity and a second tooth-receiving cavity of the one or more tooth-receiving cavities. For example, the apparatus may be configured as a palatal expander, retainer, etc. In any of these examples the sealed pressure sensor module may be positioned on the palatal region of the dental appliance. In some examples the apparatus includes a palatal expander, wherein the body comprises a palatal region extending between a first tooth-receiving cavity and a second tooth-receiving cavity of the one or more tooth-receiving cavities, wherein the sealed pressure sensor module is coupled to the palatal region.

In some examples the control circuitry receiving pressure data from the pressure sensor is configured to determine when treatment milestones are achieved. For example, the control circuitry may be configured to determine when a suture breaks based on a rate of change of sensed pressure exceeding a predetermined suture break threshold. In some examples this determination is performed locally (e.g., by the control circuitry, such as by a processor of the control circuitry). Alternatively, in some examples this is determined remotely (or shared between remote and local processors) by one or more remote processors receiving data (e.g., pressure data) from the pressure sensor module. The remote processor(s) may be cloud-based processors and/or processor of a smartphone, laptop, tablet, etc. in communication with the apparatus.

In some examples the control circuitry receiving pressure data from the pressure sensor and configured to determine the palatal expander needs to be changed based on a sensed pressure falling below a predetermined change appliance threshold.

The sealed pressure sensor module may be positioned in any appropriate position of the apparatus in order to sense pressure. As mentioned above, in some examples the pressure sensor module is positioned on the palatal region. In some examples the scaled pressure sensor module is positioned and/or included in or on a buccal or lingual side of the body. In some configurations the sealed pressure sensor may sense pressure due to airflow on or over the teeth or device; in some configurations the sealed pressure sensor may sense pressure due to saliva. In some configurations the sealed pressure sensor may sense pressure due to force acting to deflect or deform the body of the dental appliance. In any of these configurations the control circuity may be adapted to analyze the pressure data from the one or more pressure sensors encapsulated within the sealed pressure sensor module and to determine, in combination with the location of the sealed pressure sensor module, identify one or more parameters or states of the dental appliance (e.g., under stain/not under strain, damaged, etc.) and/or patient (e.g., patient health, patient sleep cycle, patient respiration, palatal expansion, etc.). Any of these apparatuses may also include one or more sensors in addition to the pressure sensor(s) within the airtight encapsulated cavity. Thus, any of these apparatuses may include one or more additional sensors within the sealed pressure sensor module. For example, an apparatus may include one or more thermal sensor(s), one or more accelerometers, one or more optical sensor(s), one or more force sensor(s), etc. The one or more additional sensors may be within the airtight encapsulated cavity, or may be external to the airtight encapsulated cavity.

In general, the airtight encapsulated cavity has a volume that is slightly larger than the volume of the one or more pressure sensors, and may be filled with a fluid (e.g., gas, liquid, etc.). The fluid may be a compressible fluid. In some examples the fluid may be an incompressible fluid. In some examples the fluid comprises air. In some examples the fluid comprises water. The airtight encapsulated cavity may have a volume of between about 0.1 cm³ and 100 cm³ in an uncompressed configuration.

The airtight encapsulated cavity may be formed of one or more materials, including one or more membranes, such as a polymeric material. The polymeric material may be elastically deformable. In some examples the polymeric material may be a synthetic or natural material (e.g., synthetic or natural rubbers), styrene-butadiene block copolymers, polyisoprene, polybutadiene, ethylene propylene rubber, ethylene propylene diene rubber, silicone elastomers, fluoroelastomers, polyurethane elastomers, and nitrile rubbers, etc. In general, the encapsulation material, and in particular the deformable surface of the encapsulation material may be formed of a polymeric material having a durometer of between about 20 and about 90 Shore 00 (e.g., less than 70 on the Shore A scale). For example, in some case the deformable surface comprises a polymeric material having a durometer of between about 0 and about 50 Shore A.

The deformable surface may have a surface area of between about 1 mm² and 1.5 cm² (e.g., between 2 mm² and 1.3 cm², between 5 mm² and 1 cm², etc.).

The sealed pressure sensor module may be integrated into and/or attached to the body of the apparatus. For example, in some cases the sealed pressure sensor module may be laminated to a body forming the one or more tooth-receiving cavities. The sealed pressure sensor module may be laminated to the palatal region. In some examples the deformable surface is configured to be on an outer surface of the dental appliance when the one or more tooth-receiving cavities are worn on the subject's teeth. In some examples the deformable surface is configured to be positioned adjacent to the subject's palate when the one or more tooth-receiving cavities are worn on the subject's teeth.

In general, the body (e.g., the tooth-receiving portion, palatal region, etc.) may comprise the same one or more fluid-resistant (e.g., in some examples, fluid-impermeable) materials as the sealed pressure sensor module.

Optionally, in any of the apparatuses and method described herein the airtight encapsulated cavity may be pressurized to an internal pressure that is greater than the surrounding pressure (e.g., within the oral cavity. For example, the airtight encapsulated cavity may be pressurized to greater than 1 atmosphere (e.g., greater than 1.1 atm, greater than 1.2 atm, greater than 1.3 atm, etc.), between 1 atm and 3 atm (e.g., between 1.1 atm and 3 atm, between 1.2 atm and 3 atm, between 1.3 atm and 3 atm, between 1 atm and 2.8 atm, between 1 atm and 2.7 atm, between 1 atm and 2.5 atm, etc.). In some variations, the pressure within the airtight encapsulated cavity may be at a pressure that is at atmospheric pressure (e.g., approximately 1 atm).

For example, a dental appliance may include: a scaled pressure sensor module including: an airtight encapsulated cavity, wherein the airtight encapsulated cavity is encapsulated by one or more fluid-resistant (e.g., in some examples, fluid-impermeable) materials; a pressure sensor encapsulated within the airtight encapsulated cavity and configured to measure pressure within the airtight encapsulated cavity; wherein at least a portion of the one or more fluid-resistant materials forms a deformable surface; a control circuitry receiving pressure data from the pressure sensor and configured to modify, store and/or transmit the pressure data; and a power source configured to apply power to the pressure sensor and the control circuity; a first tooth-receiving cavity and a second tooth-receiving cavity, wherein the first and second tooth-receiving cavities are configured to be worn on a subject's teeth; and a palatal region extending between the first and second tooth-receiving cavities, wherein the scaled pressure sensor module is coupled to the palatal region.

Also described herein are methods of making any of these apparatuses. For example, a method may include: forming a body having one or more tooth-receiving cavities configured to be worn on a subject's teeth; and coupling a sealed pressure sensor module to the body, wherein the sealed pressure sensor module includes: an airtight encapsulated cavity, wherein the airtight encapsulated cavity is encapsulated by one or more fluid-resistant (e.g., in some examples, fluid-impermeable) materials, wherein at least a portion of the one or more fluid-resistant materials forms a deformable surface; and a pressure sensor encapsulated within the airtight encapsulated cavity and configured to measure pressure within the airtight encapsulated cavity. As mentioned, the sealed pressure sensor module may include a control circuitry receiving pressure data from the pressure sensor and configured to modify, store and/or transmit the pressure data; and a power source configured to apply power to the pressure sensor and to the control circuity.

In some examples, these methods may be methods of making a dental appliance for detecting bruxism and respiration.

In any of these methods forming the body may comprise three-dimensionally printing the body. Alternatively or additionally, forming may include thermoforming the body.

Coupling may comprise laminating the scaled pressure sensor module to the body. In some examples, coupling comprises adhesively securing the sealed pressure sensor module to the body.

In any of these examples forming may comprising encapsulating the pressure sensor within the airtight cavity. Forming may comprise securing the power source to the body outside of the airtight cavity.

Any of these methods may include designing the dental appliance using a model of the subject's teeth. In particular, these methods may include designing the dental appliance using a digital model of the subject's teeth. In any of these methods, a dental treatment plan, showing multiple dental appliances for different stages or portions of a treatment plan, may be used; multiple different dental appliances may be formed, and the method may include forming a series of dental appliance including one or more pressure sensor (sensing modules) as described herein.

Also described herein are method of detecting a pressure change within the oral cavity using any of these apparatuses, including but not limited to one or more of: respiration, movement of teeth and/or suture, movement of the dental appliance itself, movement of saliva, etc. These apparatus and/or method may be configured to detect a pressure change corresponding to movement of the teeth and/or palate, including breaking or expansion of the patient's suture. In general, any of these methods may include receiving a signal from a pressure sensor within an airtight encapsulated cavity of a pressure sensor module coupled to a dental appliance being worn by a subject, wherein the airtight encapsulated cavity is encapsulated by one or more fluid-resistant (e.g., in some examples, fluid-impermeable) materials; and determining, from the signal, one or more sensed signals indicating, e.g., respiration, movement of teeth and/or suture, movement of the dental appliance itself, movement of saliva, etc. These sensed signals may be incorporated as part of a treatment, and may include alerting the patient and/or the clinician (e.g., orthodontist, dentist, etc.) based on the sensed pressure data.

For example, the sensor module may be positioned over or adjacent to the suture. In some examples, the sensor module may be positioned on or over the cavities of the teeth-receiving portions of the body of the dental appliance. The apparatus and/or method may be configured to detect pressure that the apparatus is exerting on the palate and/or teeth. In some examples the method or apparatus may be configured to detect changes in pressure on the dental appliance that indicate that the dental appliance is no longer applying a desired force on the patient's dentition (e.g., teeth, palate, etc.). The apparatus or method may be configured to detect if the dental appliance needs to be changed when the pressure detected falls below a predetermined threshold. For example, an apparatus as described herein may be configured to determine when a palatal expander needs to be changed based on the sensed pressure from a pressure sensing module as described herein, e.g., when pressure falls below a predetermined threshold resulting from a more gradual decrease/increase in force applied by the apparatus to the patient.

Also described herein are methods and apparatuses in which the pressure sensor module included with the apparatus provides data reflecting a patient's health status. The health status may be for general health (e.g., exercise, wellness, etc.) or for clinically relevant health status (e.g., detecting a disorder or disease such as a respiratory and/or cardiac disorder). In some cases the apparatus and method may be used to detect a sleep-related state, including snoring, apnea or other sleep-disordered breathing. For example, the apparatuses and methods described herein may be used to track breathing during the day to determine if baseline breathing level is good (e.g., within acceptable parameters) or bad (e.g., outside of acceptable parameters), or to determine when the patient is exercising/physically active. These methods and apparatuses may also or alternatively be used to determine breathing rate to track performance during exercise, to allow patients to keep breathing at a desired level (e.g., to keep exercise intensity within a desired heart zone), etc. Any of the apparatuses and methods described herein may be used in a clinical setting, including (but not limited to) a hospital setting. For example, any of these apparatuses may be used to tracking respiration in patients in a hospital (e.g., when breathing needs to be monitored during hospital stay) or with chronic/acute respiratory conditions (e.g., emphysema, COPD, pneumonia, COVID).

Also described herein are methods and apparatuses for detecting swallowing. For example, any of these methods and apparatuses may be configured to determine or detect saliva swallowing event/rate (when the tongue touches the palatal region and a spike in the pressure can be detected) which can be translated to salivary flow rate and body hydration/dehydration status.

For example, a method of monitoring respiration using a dental appliance may include: receiving a signal from a pressure sensor within an airtight encapsulated cavity of a pressure sensor module coupled to a dental appliance being worn by a subject, wherein the airtight encapsulated cavity is encapsulated by one or more fluid-resistant materials; and determining, from the signal, a respiration pattern.

In some examples these methods may include determining the respiration pattern by determining if the respiration pattern includes one or more of: mouth breathing, nasal breathing, snorting, and/or apnea. Determining the respiration pattern may comprise determining physical activity. In any of these examples determining the respiration pattern may comprise monitoring a patient for a respiratory disorder.

Any of these methods may include determining if the respiration pattern indicates if the subject is snoring or experiencing apnea. In some examples the method may include determining, from the signal, if the subject is experiencing bruxism.

In general, these methods may include applying or modifying a therapy based on the detected respiratory pattern. For example, these method may include adjusting a respiratory therapy based on the respiration pattern.

As mentioned, these methods and apparatuses may be configured for use with a sleeping patient. For example, these method may be configured for use when the signal is received while the subject is sleeping.

Receiving the signal may comprise wirelessly receiving the signal from the pressure sensor module. Any of these methods may include generating the signal by deforming a deformable outer surface of the pressure sensor module while the dental appliance is worn by the subject to change the internal pressure within the airtight encapsulated cavity.

Determining the respiration pattern may comprise determining if one or more frequency components of the signal is characteristic of a respiration pattern.

The method may include applying the dental appliance to the subject's mouth so that a pair of tooth-receiving portions fit onto the subject's teeth and the pressure sensor module is positioned adjacent to the subject's palate.

For example, a method of detecting bruxism and respiration may include: receiving a signal from a pressure sensor within an airtight encapsulated cavity of a pressure sensor module coupled to a dental appliance being worn by a subject, wherein the airtight encapsulated cavity is encapsulated by one or more fluid-resistant (e.g., in some examples, fluid-impermeable) materials; determining, from the signal, a respiration pattern; and determining, from the same signal, if the subject is experiencing bruxism. The respiration pattern may indicate if the subject is snoring or experiencing apnea. Determining the respiration pattern may comprise determining if the respiration pattern includes one or more of: mouth breathing, nasal breathing, snorting, and/or apnea.

Any of these methods may include adjusting a respiratory therapy based on the respiration pattern. The signal may be received while the subject is sleeping. Receiving the signal may comprise wirelessly receiving the signal from the pressure sensor module. Any of these methods may include generating the signal by deforming a deformable outer surface of the pressure sensor module to change the internal pressure within the airtight encapsulated cavity. Determining the respiration pattern may comprise determining if one or more frequency components of the signal is characteristic of a respiration pattern and/or bruxism.

The method may include applying the dental appliance to the subject's mouth so that a pair of tooth-receiving portions fit onto the subject's teeth and the pressure sensor module is positioned adjacent to the subject's palate.

Determining a respiration pattern may include using a trained neural network to determine the respiration pattern and/or if the subject is experiencing bruxism.

For example, a method of detecting bruxism and/or respiration may include: receiving a signal from a pressure sensor within an airtight encapsulated cavity of a pressure sensor module coupled to a dental appliance being worn by a subject, wherein the airtight encapsulated cavity is encapsulated by one or more fluid-resistant (e.g., fluid-impermeable) materials; determining, from the signal one or more of: a respiration pattern and if the subject is experiencing bruxism.

As mentioned, also described herein are methods of analyzing a subject's sleep, including determining if a subject has sleep apnea and/or characterizing the sleep apnea. For example, described herein are methods including: measuring an oral respiration signal using a sealed pressure sensor module coupled to a dental appliance worn on the subject's teeth; and estimating the probability of sleep apnea from the oral respiration signal.

In any of these methods, measuring the oral respiration signal may include measuring while the subject is sleeping. Estimating the probability of sleep apnea may comprise generating a representative respiratory signal for each of a plurality of subregions over time, extracting features from each representative respiratory signal, and using the extracted feature to estimate the probability of sleep apnea. For example, extracting features may comprise extracting morphological features of the representative signals. Any of these methods may include using a principle component analysis (PCA) to extract features. Using the extracted features to estimate the probability of sleep apnea may comprise identifying clusters of the extracted features corresponding to one or more of: low probability, medium probability, and high probability of apnea. In some examples, using the extracted features comprises using a trained machine learning agent to identify a probability of sleep apnea. Estimating the probability of sleep apnea may comprise aggregating a plurality of probability estimates corresponding to individual representative respiratory signals.

For example, a method of determining if a subject has sleep apnea may include: measuring an oral respiration signal using a sealed pressure sensor module coupled to a dental appliance worn on the subject's teeth; estimating the probability of sleep apnea from the oral respiration signal by generating a representative respiratory signal for each of a plurality of subregions over time, extracting features from each representative respiratory signal, and using the extracted feature to estimate the probability of sleep apnea.

Also described herein are systems configured to perform any of these methods. For example, a system may include: a dental appliance configured to be worn on a subject's teeth comprising a sealed pressure sensor module; one or more processors; and a memory coupled to the one or more processors, the memory storing computer-program instructions, that, when executed by the one or more processors, perform a computer-implemented method comprising: receiving an oral respiration signal from the sealed pressure sensor module; and estimate the probability of sleep apnea from the oral respiration signal.

For example, a system may include: a dental appliance configured to be worn on a subject's teeth comprising a sealed pressure sensor module; one or more processors; and a memory coupled to the one or more processors, the memory storing computer-program instructions, that, when executed by the one or more processors, perform a computer-implemented method comprising: receiving an oral respiration signal from the sealed pressure sensor module; and estimating the probability of sleep apnea from the oral respiration signal by generating a representative respiratory signal for each of a plurality of subregions over time, extracting features from each representative respiratory signal, and using the extracted feature to estimate the probability of sleep apnea.

Also described herein are sleep monitoring apparatuses (systems, devices, etc.). In general, these sleep monitoring systems may include the sealed pressure sensor modules described herein. These sleep monitoring apparatuses may be configured to perform any of the methods, including methods of detection pressure and/or respiration in the subject's mouth (e.g., oral cavity) and/or methods of determining if a subject has sleep apnea and/or tracking sleep apnea. The sleep monitoring apparatuses described herein can include a dental appliance that may continuously monitor changes of physiological parameters over time, including but not limited to respiration (oral pressure), temperature, etc.

In general, the apparatuses, and in particular the sleep monitoring apparatuses, described herein may include one or more of: a photoplethysmography sensor, an accelerometer, a barometer, and a temperature sensor. These sensors may be used in addition to a sealed pressure sensor module, and can be used to extract features, including (but not limited to) extracting features for sleep stage classification and sleep apnea/hypopnea detection. For example, these apparatuses, and methods of using them, may use a classification technique (e.g., a classification model) to determine sleep stage classification that uses one or more of: blood pressure or blood pressure features, heart rate, heart rate variability, respiratory rate, SpO2, sleep position, movement, and core body temperature. The methods and apparatuses described herein may determine one or more sleep parameters, including sleep latency, sleep waking, wakefulness, sleep efficiency. Sleep quality can be quantified using one or more of these sleep parameters. Apnea-hypopnea index (AHI) and oxygen desaturation index (ODI) can be calculated using the prediction results of sleep apnea/hypopnea detection and desaturation event detection algorithms.

For example, described herein are dental appliances comprising: a body forming one or more tooth-receiving cavities configured to be worn on a subject's teeth; a sealed pressure sensor module coupled to the body, the sealed pressure sensor module including an airtight encapsulated cavity that is encapsulated by one or more fluid-resistant materials, wherein at least a portion of the one or more fluid-resistant materials forms a deformable surface, and a pressure sensor encapsulated within the airtight encapsulated cavity and configured to measure pressure within the airtight encapsulated cavity; a power source; and a processor configured to receive pressure data from the pressure sensor and configured to modify, store and/or transmit the pressure data, wherein the sealed pressure sensor module, power source, and circuitry are arranged along a curve of a lingula side the body that is configured to be positioned on a lingual portion of the subject's dental arch when the one or more tooth-receiving cavities are worn on the subject's teeth.

The sealed pressure sensor module, power source, and circuitry may be within a pocket formed on the lingual side of the body. The pocket may include the airtight encapsulated cavity. In some examples the pocket may include a region comprising the airtight encapsulated cavity. As described, any of these dental appliances may include one or more temperature sensors; in some cases a temperature sensor may be located at an anterior position of the body, e.g., in a region corresponding to the canines and/or incisors when the appliance is worn by the subject. The sealed pressure sensor module, power source, and circuitry may be arranged on a substrate (e.g., a PCB substrate); in some cases this substrate may be configured to flex, including expanding and/or contracting, e.g., by including a serpentine and/or zig-zag pattern. The serpentine/zig-zag pattern may extend between left and right portion of the substrate and the left and right portion of the substrate may support the pressure sensor module and power source, respectively.

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A schematically illustrates one example of a dental appliance configured to detect respiratory rate and/or bruxism using a sealed pressure sensor module as described herein. {new para} FIG. 1B schematically illustrates an example of a pressure sensor module with wireless communication and charging circuitry.

FIG. 2A schematically illustrates an example of a dental appliance configured to detect respiratory rate and/or bruxism using a sealed pressure sensor module as described herein.

FIG. 2B schematically illustrates an example of a dental appliance configured to detect respiratory rate and/or bruxism using a sealed pressure sensor module as described herein.

FIG. 3 shows one example of a dental appliance including a sealed pressure sensor module as described herein.

FIGS. 4A-4C show bottom, top and back side views, respectively, of an example of a dental appliance including a sealed pressure sensor module similar to that shown in FIG. 3.

FIG. 5 schematically illustrates a method of forming a dental appliance as described herein.

FIG. 6 schematically illustrates a method of sensing pressure (and therefore one or both of a respiration pattern and/or bruxism) using an apparatus as described herein including a sealed pressure sensor module.

FIG. 7 illustrates the application of pressure directly to an example of a dental appliance including a sealed pressure sensor module as described herein.

FIG. 8 illustrates the indirect application of pressure (e.g., by increasing environmental pressure) to an example of a sealed pressure sensor module as described herein.

FIG. 9 shows an example of a dental appliance including a sealed pressure sensor module worn by a subject; the sealed pressure sensor monitor is concurrently transmitting a signal to a smartphone for display, storage, and/or analysis.

FIGS. 10A and 10B illustrate examples of pressure signals from a subject wearing a dental appliance including a sealed pressure sensor module; in FIGS. 10A and 10B the pressure signal indicates nasal breathing while wearing the dental appliance. Pressure is on the y-axis, time (in seconds) on the x axis.

FIG. 11 illustrates an example of a pressure signal from a subject wearing a dental appliance including a sealed pressure sensor module while breathing through the mouth. Pressure is on the y-axis, time (in seconds) on the x axis.

FIG. 12 illustrates an example of a pressure signal from a subject wearing a dental appliance including a sealed pressure sensor module while clenching the teeth. Pressure is on the y-axis, time (in seconds) on the x axis.

FIG. 13 illustrates an example of a pressure signal from a subject wearing a dental appliance including a sealed pressure sensor module while grinding the teeth. Pressure is on the y-axis, time (in seconds) on the x axis.

FIG. 14 illustrates an example of a pressure signal from a subject wearing a dental appliance including a sealed pressure sensor module while clenching and grinding the teeth. Pressure is on the y-axis, time (in seconds) on the x axis.

FIG. 15 illustrates an example of pressure within an airtight encapsulated cavity of a device during fabrication of the device using a thermoforming process.

FIGS. 16A-16F are graphs showing pressure from the pressure sensor of encapsulated pressure sensors encapsulated in sealed cavity having a relatively high internal pressure (FIGS. 16A-16B), a sealed cavity having a relatively low internal pressure (FIGS. 16C-16D), and a pressure sensor that is open to air (unsealed) (FIGS. 16E-16F), each when applying an applied pressure (FIGS. 16A, 16C and 16E) and an applied force (FIGS. 16B, 16D and 16F).

FIGS. 17A-17B show a COMSOL Multiphysics model 3D view (FIG. 17A) and cross-sectional view (FIG. 17B), including a solid body representing the encapsulated pressure sensor that is encapsulated between two deformable layers.

FIG. 18 is a graph showing a simulation of the effect of a range of pre-set internal pressures within the airtight encapsulated cavity of the simulated sealed pressure sensing module of

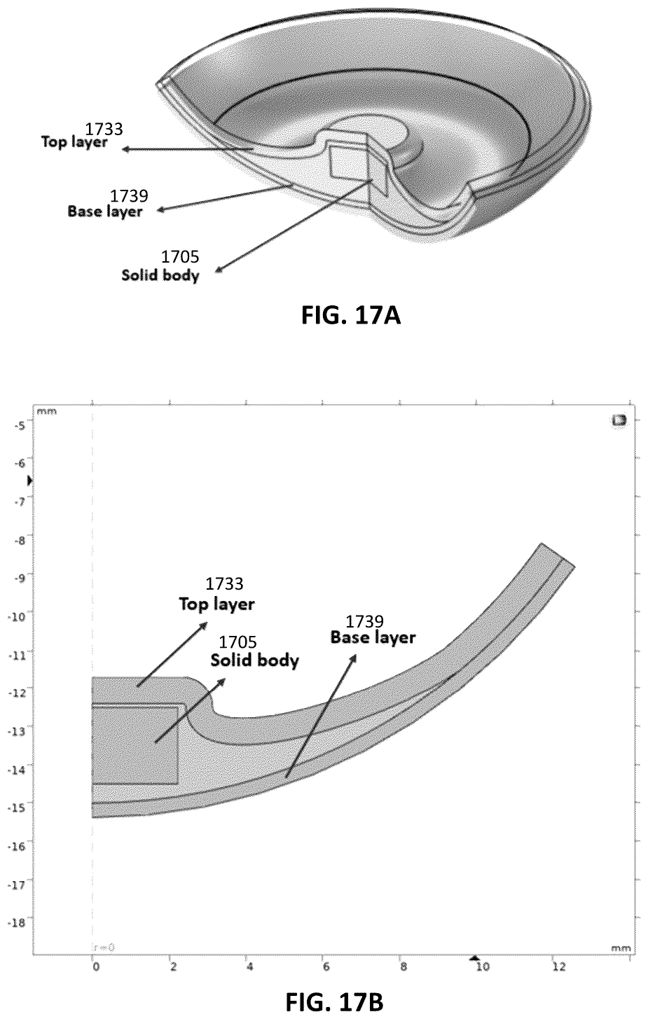

FIGS. 17A-17B. The results recorded from the COMSOL model show the cavity pressure change AP at different initial cavity pressure.

FIG. 19A schematically illustrates an example of a device including a sealed pressure sensor module configured to couple to attachments on a subject's teeth.

FIG. 19B schematically illustrates the device of FIG. 19A coupled to a subject's teeth.

FIG. 19C illustrates a device including a sealed pressure sensor module such as that shown in FIG. 19A coupled to the subject's teeth.

FIG. 20A schematically illustrates an example of a device including a sealed pressure sensor module configured to couple to a subject's teeth.

FIG. 20B schematically illustrates the device of FIG. 20A coupled to a subject's dentition.

FIG. 21 schematically illustrates an example of a device including a sealed pressure sensor module and an elastically adjustable band.

FIG. 22 schematically illustrates an example of a device including a sealed pressure sensor module and an elastically adjustable band.

FIG. 23 schematically illustrates the device of FIG. 21 attached to a subject's teeth.

FIGS. 24A-24D illustrate another example of a dental appliance including a sealed pressure sensor module as described herein. FIGS. 24B-24D show schematic illustrations of a top, bottom and side view, respectively, of a dental appliance including a sealed pressure sensor module.

FIGS. 25A-25B are graphs illustrating pressure measurements taken using a dental appliance including a sealed pressure sensor module when the subject is wearing the dental appliance and is ether breathing through the nose (FIG. 25A) or through the mouth (FIG. 25B).

FIG. 26 illustrates an example of a pressure signal from a subject wearing a dental appliance including a sealed pressure sensor module while clenching the teeth (three times) and grinding the teeth (three times). Pressure (left side) and force (right side) are shown on the y-axis, time (in seconds) on the x axis.

FIG. 27 is a graph illustrating temperature sensed with a dental appliance in which the temperatures sensor is positioned anteriorly and on a lingual side of the dental appliance (e.g., behind a canine) while the subject wearing the dental appliance is breathing through their mouth.

FIG. 28A schematically illustrates an example of a method of detecting apnea and determining apnea probability.

FIG. 28B illustrates an example of a method of determining characteristic features of a respiratory signal and reducing the dimensionality of the signal.

FIGS. 29A-29C illustrate one example of a method of generating a characteristic breathing pattern (e.g., a single characteristic ‘pulse’) including inspiration and expiration over an epoch (i.e., a time window).

FIG. 30 illustrates an example method where single pulses are generated separately for each of multiple phases of breathing (e.g., inspiration and expiration) and then concatenating them into a single combined pulse that includes information about the multiple phases (e.g., inspiration and expiration).

FIG. 31 illustrates one example of a method of reducing dimensionality of a single characteristic pulse.

FIG. 32A illustrates an example of mapping pulses on a feature space and clustering the pulses based on their mapped position in the feature space during a training phase using a supervised learning method.

FIG. 32B illustrates an example process of training a model to label identified clusters (e.g., labeling them with apnea probability values) based on the number of apnea and hypopnea events within each cluster.

FIG. 32C shows an example of identifying and labeling clusters corresponding to apnea probability.

FIG. 33 schematically illustrates an example of a technique for predicting the probability for apnea in a sleeping subject using clustering.

FIGS. 34A-34D show graphs illustrating the probabilities of a subject's apnea using the methods described here, e.g., in FIG. 28A. FIG. 34A illustrates the probabilities of a subject's apnea over time by characterizing individual 30-second ‘epochs’ as low, medium, high, and very high probability of apnea. FIGS. 34B and 34C show apnea probabilities over an 8 hour sleep period as a bar graph and pie chart, respectively. FIGS. 34D shows apnea probability correlated to body position (e.g., which may be measured by an accelerometer on the dental appliance) during an 8 hour sleep period.

FIGS. 35A-35B illustrate detection of respiratory rate. FIG. 35A schematically illustrates one example of a method of detecting respiratory rate. FIG. 35B is an example of a spectrogram of pressure that may be used to determine respiratory rate.

FIGS. 36A-36B illustrate detection of mouth breathing (and/or nose breathing). FIG. 36A schematically illustrates one example of a method of detecting mouth/nose breathing. FIG. 36B is an example of the detection of mouth/nose breathing.

FIGS. 37A-37C illustrate another example of the detection of sleep apnea. FIG. 37A schematically illustrates feature extraction from an intraoral pressure signal. FIG. 37B are graphs illustrating one example of preprocessing and feature extraction from the pressure signal. FIG. 37C schematically illustrates one example of training and applying a model for interpreting features extracted from the intraoral pressure signal to detect sleep apnea, and/or to classify the sleep apnea.

FIG. 38 schematically illustrates a method of determining sleep stages using an apparatus as described herein.

FIGS. 39A-39B show an example of determination of sleep position. FIG. 39A schematically illustrates a method of detecting sleep position. FIG. 39B shows examples of types of movement that may be extracted from accelerometer data provided by an apparatus as described herein.

FIG. 40 schematically illustrates one example of a method of detecting core body temperature using an apparatus as described herein.

FIG. 41 is an example of detection of bruxism using an apparatus as described herein.

FIG. 42 schematically illustrates examples of categories of sleep stage classifications that may be used with four exemplary sensors for any of the dental appliances described herein that may sense pressure (using a sealed pressure sensing module), temperature, barometer, and/or photoplethysmography.

FIG. 43 schematically illustrates the combined use of combinations or sub-combinations of multiple sleep detection models (e.g., sleep stage classification, sleep apnea detection, sleep position classification, mouth breathing detection, etc.) to provide output relevant to predicting and/or diagnosing a patient's sleep apnea.

### DETAILED DESCRIPTION

Described herein are methods and apparatuses (e.g., devices and systems, including hardware, software, and firmware) for sensing pressure and for determining the status of a patient, dental appliance, and/or a treatment. In general, these apparatuses may include a dental appliance having one or more sealed pressure sensor modules as described herein. These sealed pressure sensor modules are configured to detect pressure using one or more pressure sensors within the sealed (e.g., airtight) encapsulated cavity that may be filled with a fluid. The sealed pressure sensor module includes one or more elastically deformable surfaces surrounding the encapsulated cavity, and the pressure within the encapsulated cavity (“internal pressure”) may change as a result of deformation of one or more elastically deformable surfaces. This internal pressure may be detected and used to derive pressure outside of the sealed pressure sensor module (“external pressure”), for example, a pressure in the intraoral cavity when the pressure sensor module is within the intraoral cavity. The one or more elastically deformable surfaces may be configured to be sensitive to external pressure such that they deform based on the amount of external pressure. The external pressure may include the air pressure within the oral cavity and/or mechanical force applied onto the dental appliance to which the sealed pressure sensor module is attached. Air pressure is the force exerted by air, while mechanical force (or mechanical pressure) may be force applied by one or more object's, such as the movement of the jaw or tongue, acting on the scaled pressure sensor module directly or indirectly (e.g., acting on the body to which the pressure sensor module is coupled). In some embodiments, deformation of the elastically deformable surface(s) changes the volume of the internal airtight encapsulated cavity, which thus causes the internal pressure to change. The internal pressure is therefore related to the external pressure, such that the external pressure can be derived based on a measured internal pressure. The configuration of the scaled pressure sensor module may be more, or selectively, sensitive to external pressure, including amplifying and/or filtering the external pressure. In some examples the amount of deformation of the one or more deformable surfaces can be measured, e.g., by resistivity or strain gauge, and the amount of deformation can be used to supplement or derive external pressure. Alternatively or additionally, an acoustic sensor can be used to detect sound waves instead of measuring pressure for the same applications.

The process of translating pressure through the deformable surface(s) of the sealed pressure sensor module may modify the sensed external pressure so that it may be reliably and readily transduced by the one or more pressure sensors within the sealed encapsulated cavity, e.g., normalizing, filtering, and/or amplifying the sensed external pressure. For example, it may be easier to detect an external pressure using the encapsulated cavity since the encapsulated cavity may amplify sensed changes in external pressure. Thus these apparatuses may be highly sensitive. The combination of a dental appliance and one or more sealed pressure sensor module allows for mechanical pressure sensing using a sensor (such as an air pressure sensor), which allows a variety of previously difficult to reliably monitor physiological signals and actions to be monitored reliably and continuously/semi-continuously for an extended period. In addition, the scaled pressure sensor module may isolate and protect the pressure sensor from the environment of the oral cavity, which may otherwise be deleterious to the sensor.

Also described herein are methods of making these sealed pressure sensor modules and method of using them. For example, described herein are methods of using sealed pressure sensor modules to detect pressure data and analyzing the pressure data to determine a state of the associated dental appliance and/or the subject wearing the dental appliance. The location and orientation of the scaled pressure sensor module relative to the dental appliance may allow the detection of pressure related to specific states of the dental appliance, subject (e.g., patient) and/or treatment plan. For example, the methods an apparatuses described herein may orient the sealed pressure sensor module so that it detect forces acting on the dental appliance, including force applied to move teeth, palate, etc. As another example, pressure changes determined based on sensor readings may be used to determine when the appliance is in use (e.g., when the appliance is within the oral cavity), and track/log such usage. Thus, the scaled pressure sensor module may be used to track compliance. In some variations the scaled pressure sensor module may be oriented and configured so that it detects pressure changes due to respiration.

In any of these apparatuses and methods, the pressure sensor module may detect pressure from respiration, speaking (e.g., lingual contact force on hard palate during speech), and/or from biting (e.g., bruxism, eating/chewing, etc.). Although the one or more pressure sensors within the encapsulated cavities may be fully sealed off from atmosphere in a deformable airtight cavity coupled with and/or integrated into the dental appliance, these sealed pressure sensor modules may have numerous benefits and advantages for intraoral applications. The use of one or more pressure sensors sealed within an airtight encapsulated cavity may, in effect, form a separate inner pressure chamber that will still be sensitive to fluctuations in external pressure due to the deformability of the encapsulation material, transmitting pressure to the fluid (e.g., air) that is within the encapsulated inner cavity or chamber. This configuration may result in sensor output corresponding to an internal pressure, Pᵢₙₜₑᵣₙₐₗ, that may be described as:

$${P}_{internal} = {P}_{baseline} + Δ P \qquad [ 1 ]$$

where P_{baseline} is a baseline pressure of the encapsulated cavity and AP is an amount of pressure that may be provided by, for example, the external pressure of the oral cavity and/or pressure applied by force acting on the dental appliance to which the scaled pressure sensor module is attached. In some examples, this internal pressure may then be used to derive an external pressure based on the relationship between the two values, as explained in further detail below. In some examples, one or more calibration steps may be performed (e.g., while the subject wears the pressure sensor module) to allow for deriving more accurate external pressures. Advantages of this configuration may include having a lower complexity in the final apparatus design and construction, using less materials and processes, etc. For example, these apparatuses may not need venting material (unlike the more conventional vented pressure sensors). These apparatuses may also have improved moisture resistance, especially in the face of moisture condensation and full submersion of the device, for example, when it is placed in an oral cavity or cleaned. In a conventional vented pressure sensor, such conditions could block the vent and allow negative pressure to draw moisture into the electronics housing. These apparatuses may also be significantly safer, e.g., due to less moisture ingress and a reduction of potential leachants. In addition, these apparatuses may transduce mechanical forces (including stress and/or strain on the dental appliance) into an appreciable pressure, allowing recording of physiological signals not previously detectable using conventional pressure sensors (e.g., sensing lingual contact pressure on the hard palate during speech). In some of these examples the apparatus may include one or more pressure sensors within the airtight encapsulated cavity, and may be coupled to a controller (control circuitry), a wireless communications circuitry (e.g., BLE chip) for wireless communication, and a power source (e.g., battery for standalone power). These components may be fully scaled between layers of materials of a dental appliance (e.g., aligner, retainer, palatal expander, mouth guard, etc.) as described below. The output from the sealed pressure sensor module may be communicated wirelessly to a remote device, such as (but not limited to) a mobile device (e.g., a mobile phone), a remote server, or other computing device running a custom software (e.g., a mobile app) for receiving, processing, transmitting and/or displaying the pressure data or an output based on the pressure data. The output may be part of a user interface.

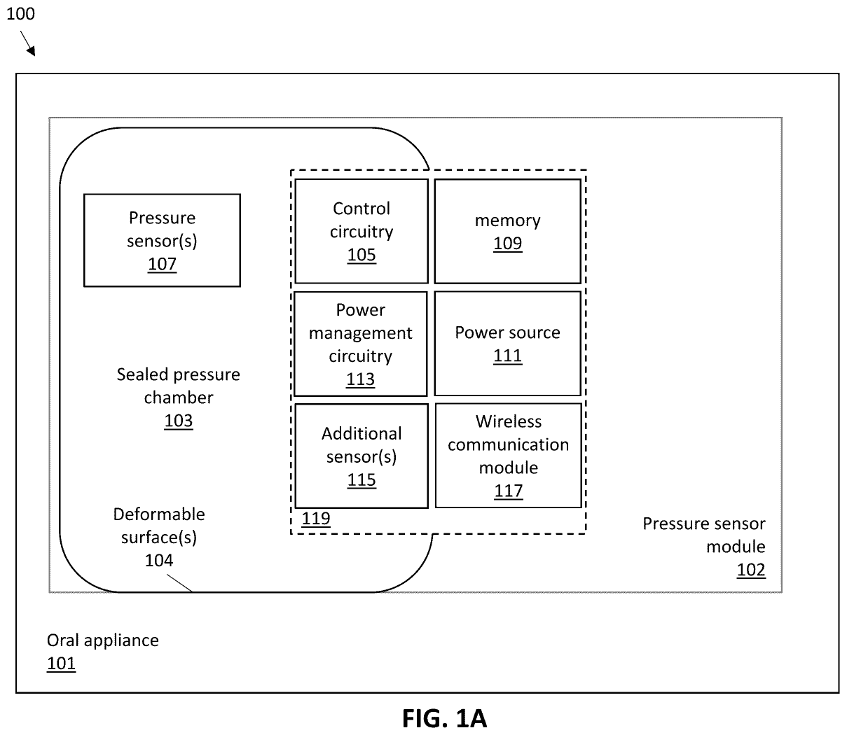

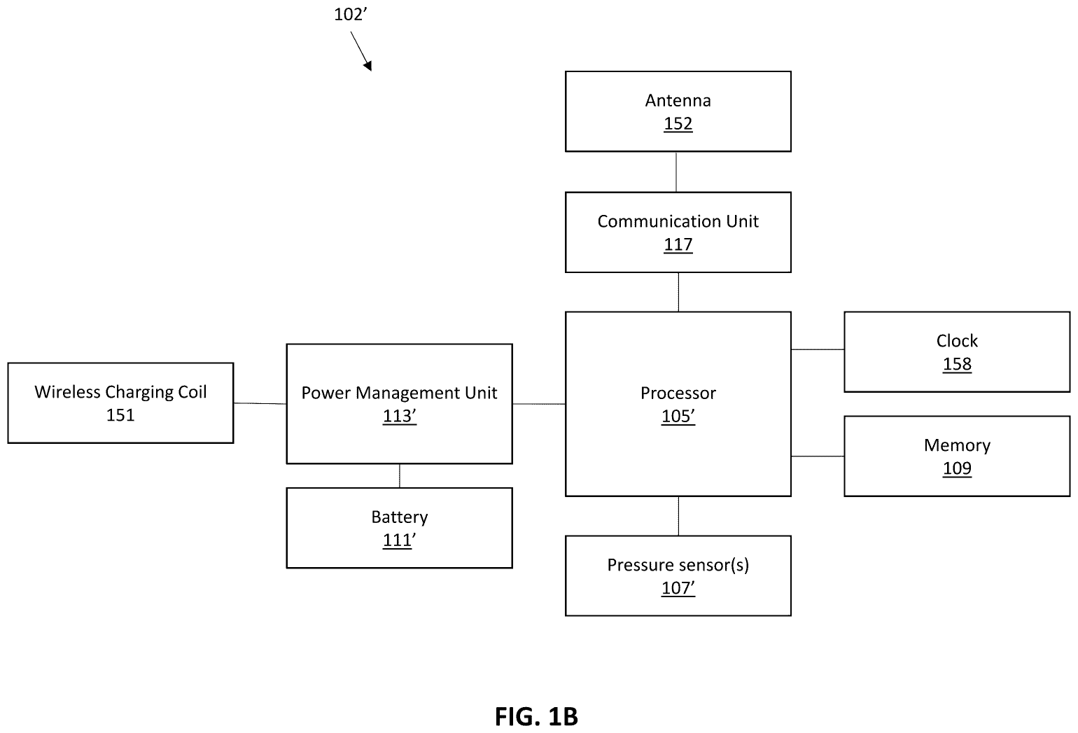

FIG. 1A schematically illustrates a first example of an apparatus 100 as described herein, including an oral appliance 101 (e.g., aligner, retainer, palatal expander, mouth guard, etc.) to which a pressure sensor module 102 is coupled. The pressure sensor module may include a sealed encapsulated cavity 103 configured as an airtight cavity enclosing one or more pressure sensors 107 and a fluid (e.g., air, a liquid, a compressible gel). The scaled pressure sensor may include one or more deformable surfaces 104 that may be continuous with an outer surface of the pressure sensor module.

The one or more pressure sensors scaled within the sealed pressure chamber may be any appropriate pressure sensor. For example, the pressure sensor(s) may be a resistive pressure sensor that detects a change in electrical resistance (e.g., of a strain gauge bonded to a diaphragm exposed to the fluid within the scaled pressure chamber 103). The resistive pressure sensor may include a strain gauge member comprising a metal resistive element on a flexible backing bonded to a diaphragm, or deposited directly using thin-film processes. In some examples the strain gauge element can be deposited on a ceramic diaphragm using a thick-film deposition process. In some examples, the pressure sensor may be a piezoresistive sensors configured to detect a change in resistivity of semiconductor materials when subjected to strain due to deflection of a diaphragm element. In some examples, the pressure sensor(s) may be a capacitive pressure sensor. The pressure sensor(s) may be an optical pressure sensor, which may use, for example, interferometry to measure pressure-induced changes in optical fiber. In some examples the pressure sensor(s) may be a MEMS (Micro Electro-Mechanical System) sensor fabricated on silicon. In some examples more than one pressure sensor may be used; in particular, multiple types or categories of pressure sensors (e.g., optical, piezoelectric, resistive, etc.) may be used. Alternatively, the same type or category of pressure sensor may be used but having different dimensions and/or sensing ranges. As mentioned, the pressure sensor may be any appropriate pressure sensor, including an acoustic sensor. In some examples the pressure sensor encapsulated within the airtight encapsulated cavity comprises a barometric pressure sensor.

The sealed pressure chamber 103 generally encloses the one or more pressure sensors, at least partially, so that pressure within the sealed pressure chamber may be detected by the one or more pressure sensors. In some cases, one or more walls of the sealed pressure chamber may be configured as a deformable surface 104 to transduce a pressure outside of the pressure sensor module into a change in pressure of the fluid (e.g., air) within the airtight and sealed pressure chamber. The deformable surface may deform so as to change the pressure of the fluid within the encapsulated cavity. The deformable surface may be formed as part of the outer covering of the sealed pressure chamber 103 and/or the pressure sensor module 102. In some cases, the deformable surface may be a polymeric material that is elastically deformable. For example, the sealed pressure chamber, and in particular the deformable surface) may be formed of a polymeric material that may include a synthetic or natural material (e.g., synthetic or natural rubbers), a styrene-butadiene block copolymer, a polyisoprene, a polybutadiene, an ethylene propylene rubber, an ethylene propylene diene rubber, a silicone elastomer, a fluoroelastomer, a polyurethane elastomer, a nitrile rubber, etc., or some combination of these. The encapsulation material forming the sealed pressure chamber and/or just the deformable surface may be formed of a polymeric material having a durometer of between about 20 and about 90 Shore 00 (e.g., between about 0 and about 70 on the Shore A scale). For example, in some case the deformable surface comprises a polymeric material having a durometer of between about 0 and about 50 Shore A.

The sealed pressure chamber 103 may be any appropriate volume, such as, e.g., between about 0.1 cm³ and 100 cm³ (e.g., between about 0.2 cm³ and about 70 cm^{3, 0.5} cm³ and about 50 cm^{3, 0.1} cm³ and about 40 cm^{3, 0.1} cm³ and about 30 cm^{3, 0.1} cm³ and about 20 cm^{3, 0.1} cm³ and about 10 cm^{3, 0.2} cm³ and about 7.5 cm³, etc.). Similarly, the deformable surface(s) may have an aggregate surface area of between about 1 mm² and 50 cm² (e.g., between about 1 mm² and 40 cm², between about 1 mm² and 30 cm², between about 1 mm² and 25 cm², between about 1 mm² and 20 cm², between about 1 mm² and 15 cm², between about 1 mm² and 10 cm², between about 1 mm² and 7.5 cm², between about 1 mm² and 5 cm², etc.). The deformable surface may be an outer surface of the pressure sensor module, and may be an outer surface of the oral appliance (e.g., for contacting the teeth, gingiva, and/or palate), or in some examples, for facing into the oral cavity away from the teeth, gingiva and/or palate. In some examples the deformable surface may be in contact with the material forming the body of the dental appliance.

The pressure sensor module 102 may also include a controller comprising control circuitry 105. The controller may include or may be operationally coupled with one or more processors, a memory 109 (e.g., for storing pressure data received by the pressure sensor(s) 107) signal processing circuitry (e.g., for receiving and/or processing pressure data and/or data from other sensors 115), a wireless communication module 117 (e.g., for transmitting/receive signals using a wireless transceiver), and/or power management circuitry 113 (e.g., for regulating power to the sensor(s) and/or other components, including the controller). The pressure sensor module may also include one or more power sources 111. The power source may include a power store, such as one or more of a battery, a capacitor, or the like. In some examples, the power may be received inductively, and may be used as received and/or may be stored (e.g., recharging the power store).

The circuitry 119, including the control circuitry, may be enclosed within the sealed pressure chamber 103 or all or some of it may be separate from the sealed pressure chamber, but in electrical communication with the one or more pressure sensors 107.

Thus, these apparatuses may include any of the sensors described herein, e.g., one or more thermal/temperature sensor(s), one or more accelerometers, one or more optical sensor(s), one or more force sensor(s), etc., In particular, these apparatuses may include one or more photoplethysmography (PPG) sensors, one or more sealed pressure sensors, one or more temperature sensors, and one or more accelerometers. These one or more additional sensors may be positioned on the dental appliance within the sealed pressure chamber or outside of the sealed pressure chamber, as indicated in FIG. 1A by the dashed box.

The pressure sensor module may be fully sealed, e.g., to prevent fluid (e.g., saliva) ingress/egress, or may be partially sealed. For example, the power store may be in electrical communication with the sealed portions of the pressure sensor module but may be separated therefrom and not sealed, to allow removal/recharging of the power store (e.g., battery). In other embodiments, the power store may be sealed along with the other portions of the sensor module (e.g., within the encapsulated cavity).

FIG. 1B schematically illustrates an example of a pressure sensor module 102′ with wireless communication and charging circuitry. In this example the pressure sensor module 102′ includes a controller portion with one or more processors 105′, a clock 158, a memory 109, a power management unit 113′ and a communication unit 117. The communication unit may include one or more antennas 152 or the one or more antennas may be separate. The power management unit 113′ may be included as part of the controller or it may be separate. One or more wireless charging coils 151 may be included as part of the power management unit 113′ or it may be separate. The pressure sensor module may also include a power store (e.g., battery 111′). The power store and/or wireless charging coil may be managed by the power management unit which in turn may be controlled by the controller. The pressure sensor module 102′ may also include one or more pressure sensor(s) 107′ encapsulated within a sealed pressure chamber 103 (not shown in FIG. 1B).

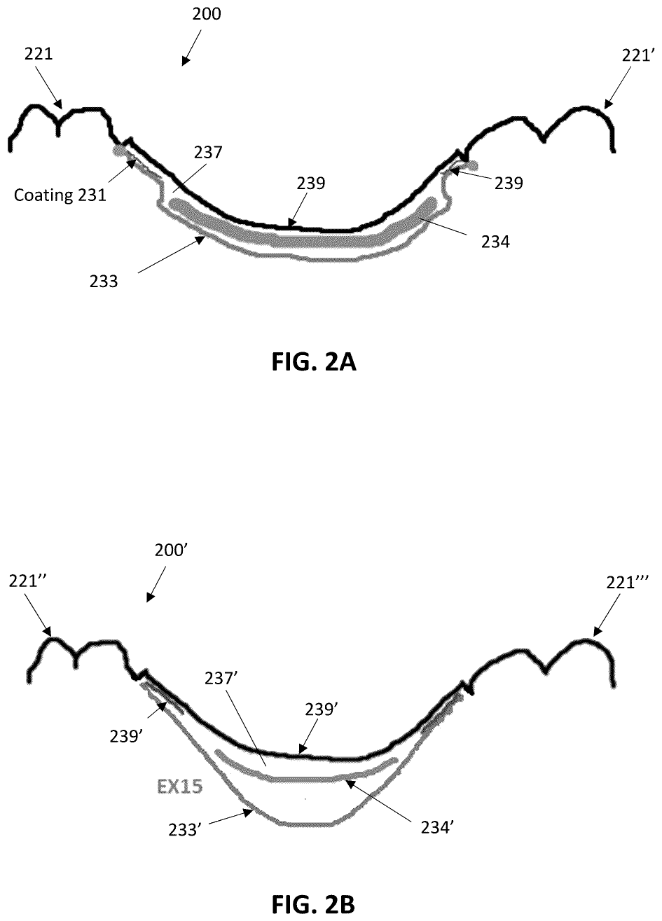

FIGS. 2A and 2B illustrate examples of a dental appliance 200, 200′ having a sealed pressure sensor module integrated into the dental appliance. In FIG. 2A, the dental appliance includes a body having a pair of tooth-receiving portions 221, 221′ and a palatal region 239 spanning between the tooth-receiving portions. The palatal region may be rigid or may include a deformable membrane or layer. The dental appliance 200 includes a scaled pressure sensor module that includes electronics 234 (e.g., control circuitry, pressure sensor, etc.) within an encapsulation cavity 237. The encapsulation cavity 237 is formed by laminating an outer elastically deformable surface 233 to the palatal region of the dental appliance by lamination region 239. In FIG. 2A the outer elastically deformable surface 233 (shown as an elastically deformable layer of material) forming one side of the encapsulation cavity 237 is coupled with an adhesive layer 231 (e.g., Dymax 1187-M) securing the elastically deformable surface 233. In FIG. 2A the elastically deformable surface is formed of a thermoplastic elastomer (e.g., polyurethane). The elastically deformable material may be relatively thin (e.g., in some examples between 0.010 and 0.020 inches) as compared with the body portion, such as the palatal region of the dental appliance (e.g., 0.025 to 0.035 inches thick). The body of the dental appliance may be formed of a biocompatible polymeric material, such as a polyester material (e.g., polystyrene). In some examples, as shown in FIG. 2A, the deformable surface may be configured to be positioned against or opposite from the palatal region when the apparatus is worn by the patient.

FIG. 2B shows another example of a dental apparatus 200′ similar to that shown in FIG. 2A, in which the apparatus includes also include a pair of tooth-receiving regions 221″, 221″″ and a palatal region 239′; the pressure sensor module is coupled to the palatal region by laminating the elastically deformable surface 233′ to the outer upper surface of the palatal region at the lamination region 239. The electrical components 234′, including the pressure sensor module is secured within the airtight encapsulated cavity 237′. In FIG. 2B an additional adhesive is not used, but the elastically deformable surface 233′ forming the fluid-resistant (e.g., fluid-impermeable) outer covering of the encapsulation cavity may be directly laminated to the body of the dental appliance. This may be achieved by selecting the materials forming the body of the dental appliance and/or the encapsulation cavity (e.g., the deformable surface 233′) so that they have properties allowing them to be directly laminated. In FIG. 2B the material forming the elastically deformable surface 233′ may be, for example, a poly (lactic acid) (PLA) material.

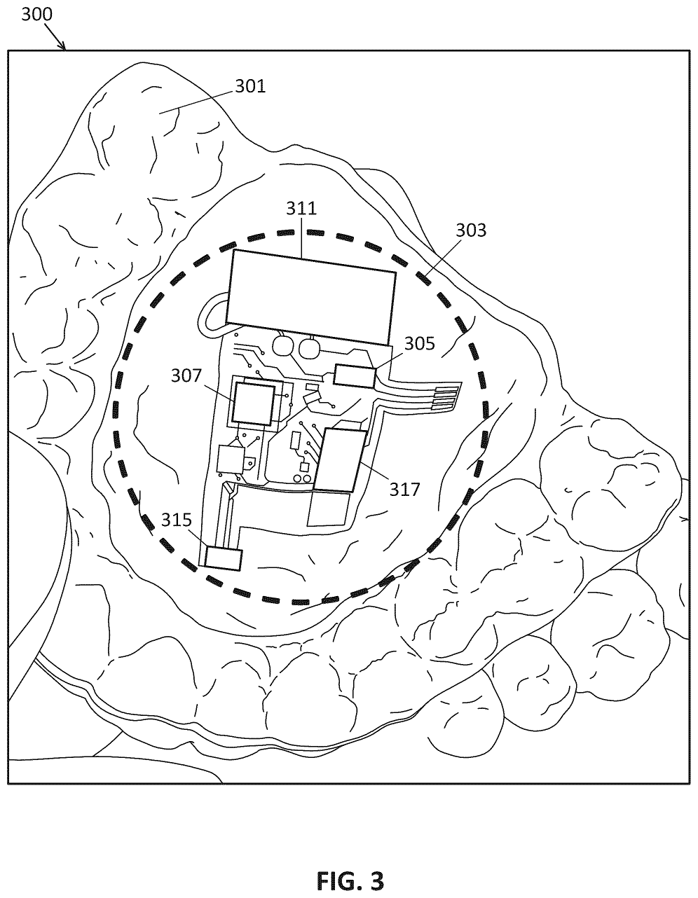

FIG. 3 illustrates one example of a dental appliance 300 including a sealed pressure sensor module that is laminated to a palatal region, similar to those shown in FIGS. 2A-2B, above. In this example the dental appliance includes a tooth-receiving cavity of the oral appliance 301. The scaled cavity is formed by laminating a deformable surface to the palatal region to enclose the pressure sensor electronics. For example, the electronics (e.g., pressure sensor 307, battery 311, control circuitry 305, BLUETOOTH communications module 317, etc.) are encapsulated within the scaled chamber 303. The pressure sensor module may also include one or more additional sensors 315 (e.g., an accelerometer). In FIG. 3, the dental appliance includes a body configured as an aligner. As in FIG. 2A, the deformable surface may be laminated between thermoformed plastic layers and may (e.g., using a light-cured adhesive) create an airtight seal around the pressure sensing electronics with air (e.g., a compressible fluid) within the cavity. Pressure applied to the sealed cavity may be transduced by the one or more pressure sensors. In FIG. 3, the deformable surface may be configured to detect pressure from the subject wearing the apparatus to detect even small changes in intraoral pressure caused by actions like swallowing and breathing. Another example of a dental appliance including a scaled pressure sensor module is shown in FIGS. 24A-24D, described in greater detail below.

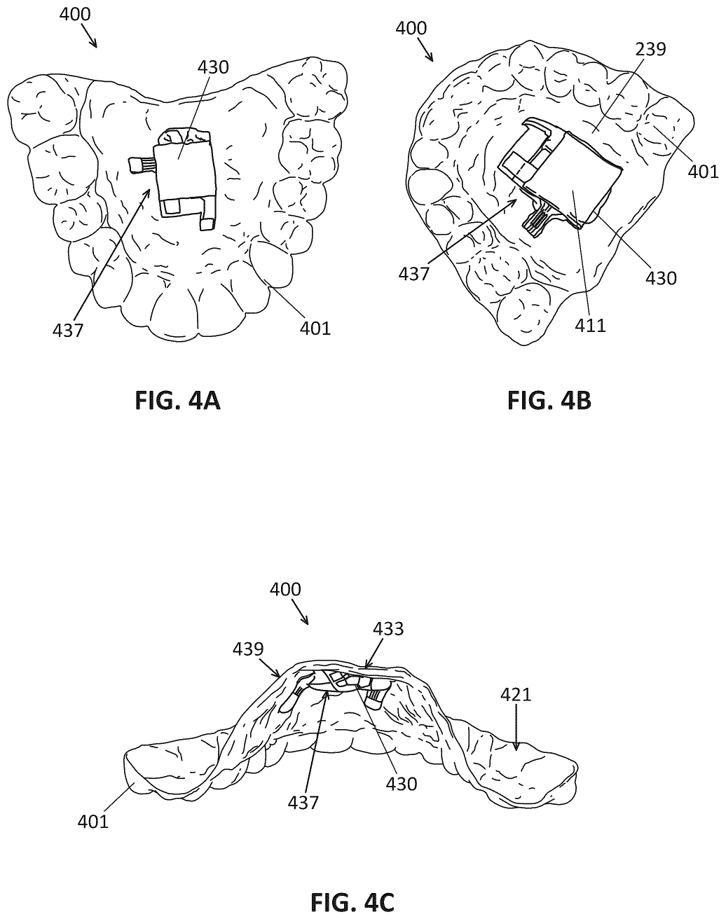

FIGS. 4A-4C illustrate another example of a dental appliance 400 configured as a retainer including a sealed pressure sensing module 430 secured to, or formed as part of, a palatal region of the dental appliance 400. In some examples, the pressure sensor module may be coupled to the tooth-receiving portion of the body of the dental appliance. Note that in some examples the dental appliance does not include a palatal region at all, and the pressure sensor module may be coupled to the tooth-receiving portion of the body of the dental appliance. The electronics, including the one or more pressure sensors, are sealed within the encapsulation cavity 437 and protected from the oral cavity environment. In FIG. 4A, the pressure sensing module is laminated to the palatal region so that the deformable surface or layer 433 is exposed on the bottom side of the apparatus so that it may detect pressure within the airway inside of the oral cavity. In some examples the deformable surface may be positioned against (or integrated with) the body of the dental appliance (e.g., to detect strain or forces as pressures acting on or through the dental appliance). The body of the dental appliance 400 (e.g., configured as a retainer) shown in FIGS. 4A-4C is transparent, so that the encapsulation cavity 437 and encapsulated pressure sensor and pressure sensing module 430, and battery 411 are visible. The body 401 of the retainer includes a tooth-receiving cavity 421.

In FIGS. 4A-4C the apparatus includes pressure sensor module circuitry having a printed circuit board (PCB) to which the components, including the battery and pressure sensor, are coupled. This example may be configured for sleep monitoring, including detection of bruxism (e.g., teeth grinding) and/or respiration. For example, the apparatus shown in FIGS. 4A-4C can be configured to measure and/or analyze the subject wearing the apparatus's breathing pattern, and may distinguish between nose/mouth breathing, detect apnea, snoring, etc. For example, the controller of the apparatus (or a separate processor in communication with the pressure sensor module) may analyze the pressure date and may detect or determine one or more of: respiration, tooth grinding, tooth clenching, snoring, etc.

### Methods of Making

In general, the pressure sensor module may be coupled with or incorporated into a dental appliance having a body that includes one or more tooth-receiving cavities. The dental appliance may also include a palatal region. In some examples an apparatus as described herein may be formed by integrating the components of the pressure sensor module into the body of the dental appliance. As mentioned above, in some examples at least the one or more pressure sensors may be sealed within an airtight encapsulated cavity that may be filed with a fluid (pressure transduction fluid); in some examples the fluid may be a gas, e.g., CO₂, nitrogen, air, or some other combination of gases. In some examples the fluid may be liquid (e.g., water, such as saline or distilled water, etc.). The fluid may be compressible or incompressible. In some examples, the pressure sensor may be embedded in an over-molded or cast housing and can be directly mounted on the surface of an intraoral device. In some examples the sealed pressure sensor module may then be formed by securing (e.g., adhesively, welding, laminating, etc.) a cover, which may include the elastically deformable surface(s) over region in which the pressure sensor is attached, to form the airtight encapsulated cavity. In general, the term “cavity” may be used to describe any enclosed space within or on the body of the dental appliance. Alternatively, in some examples a sealed pressure sensing module that is already enclosed may be coupled (adhesively coupled, welded, etc.) to the body of the dental appliance. In any of these methods the controller (e.g., control circuitry) and electrical components (battery, additional memory, etc.) may be secured within the airtight encapsulated cavity, in a separate cavity, or separate from the airtight encapsulated cavity. In some examples, some of these components are within the airtight encapsulated cavity with the pressure sensor(s) and some are not. In other examples, all of these components are within the same airtight encapsulated cavity.

In some examples the pressure sensor module can be embedded in a 3D printed or additively manufactured dental appliance as part of the direct fabrication process, or separate from the direct fabrication process. For example, a direct fabrication form factor could allow for direct integration of electronics in 3D printed dental appliances. In some examples an additively manufactured material could form the airtight cavity for this application, allowing for further improvements to manufacturability.

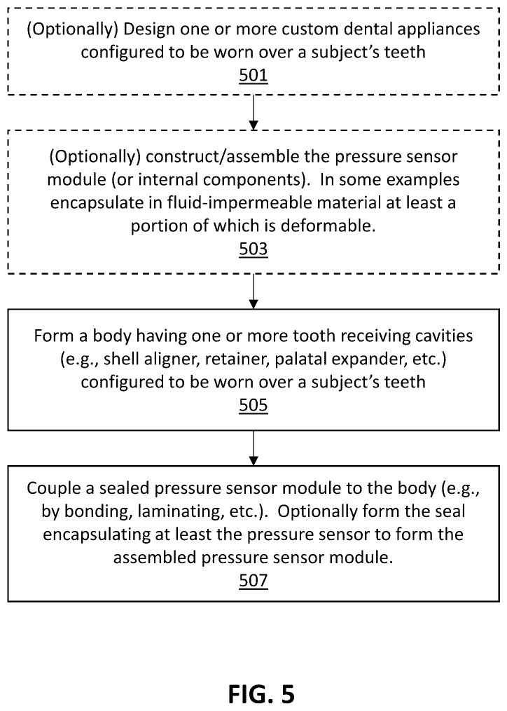

FIG. 5 illustrates one example of a method for fabrication of an apparatus as described herein. In FIG. 5, a plan or design for a dental appliance may be optionally formed as part of the process. For example, the method may include an optional step of designing one or more custom dental appliances configured to be worn over a subject's teeth 501. The design may be performed using software (e.g., digitally). In some examples, the design process may include designing a treatment plan with one or more treatment steps for modifying a patient's dentition.

In some examples the method of forming the dental appliance may also include constructing and/or assembling the pressure sensor module (or a subset of the pressure sensor module). This may include coupling the components to a PCB, multiple PCBs, and/or to a frame or bonding substrate for coupling to the body of the dental appliance 503. In some examples the method may include encapsulating all or some of the pressure sensor module components within an airtight encapsulated cavity having a fluid-impermeable material at least a portion of which is deformable surrounding the pressure sensor.

The method shown in FIG. 5 may include forming the body of the dental appliance (or in some examples, receiving the formed body) 505. The body may have one or more tooth-receiving cavities configured to be worn over a subject's teeth. The body may be configured as a shell aligner, retainer, palatal expander, mouth guard, etc. In any of these methods, the body may be prepared to receive the pressure sensor module, for example, by attaching an adhesive material, forming a portion of the cavity, reinforcing the attachment region, etc.

The method may further include coupling the pressure sensor module to the body (e.g., by bonding, laminating, etc.) 507. Optionally, this step may include forming the seal encapsulating at least the pressure sensor to form the assembled pressure sensor module, e.g., by scaling a layer (which in some examples include the deformable surface) over the components of the pressure sensor module. In some examples this step forms the airtight encapsulated cavity. Attaching the sealing layer may include filling the formed airtight encapsulated cavity with the fluid (e.g., air, water, etc.). The dental appliance may then be post-processed, e.g., to smooth, texture, cure, etc. In some examples, steps 505 and 507 may be performed simultaneously (e.g., forming the encapsulated cavity (with the pressure sensor module therein) on the body as the body is being formed).

### Methods of Use

The apparatuses described herein may be used to monitor a patient; monitor progress or effectiveness of a treatment (including but not limited to a dental/orthodontic treatment, a sleep disorder treatment, etc.); detect, monitor, and/or diagnose a condition (e.g., sleep apnea, bruxism); track overall health or performance; and/or monitor a condition or effectiveness of a dental appliance as it is worn by a patient. These methods may be performed in part by the apparatus, e.g., by the controller (control circuitry) of the apparatus, including by software (e.g., computer-implemented instructions, such as non-transitory computer-readable medium including contents that are configured to cause one or more processors to perform the method). The methods may be performed in part by a computing device separate from the apparatus (e.g., a processor of a mobile device, a remote server).

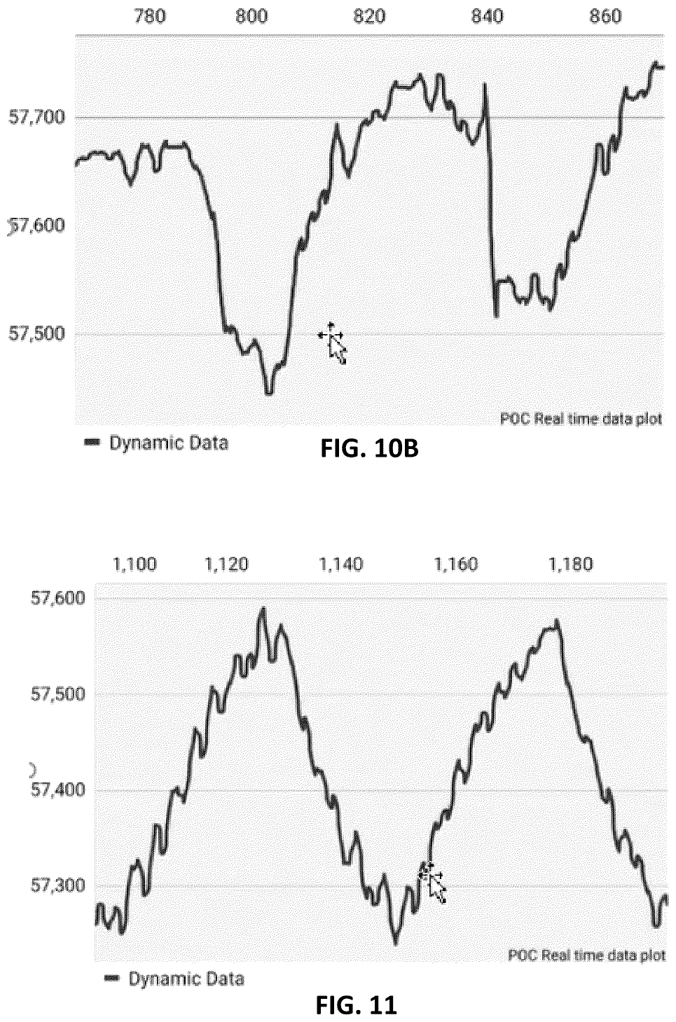

For example, the methods and apparatuses described herein may be configured to monitor respiration. The apparatuses and methods described herein may include one or more sealed pressure sensor modules that are arranged on a dental appliance (e.g., retainer, mouthguard, palatal expander, aligner, etc.) so that intraoral pressure and/or changes to intraoral pressure due to breathing (e.g., mouth and/or nasal breathing) may be detected. For example, a deformable surface of the encapsulating cavity of the sealed pressure sensor module may be positioned on an outward-facing surface of the dental appliance (e.g., a surface that faces the oral cavity when worn). As the subject breathes the air pressure may deflect the deformable surface, resulting in a detectable pressure change on the fluid within the encapsulation cavity of the sealed pressure sensor module. The controller may receive, and may pre-process the pressure data and/or may store, process, and/or transmit the pressure data to a remote processor. The pressure data may be analyzed to determine the pattern of respiration. This data may be used to determine the rate of breathing (respiration) and to infer patient health or activity based on the analyzed respiration. Nasal breathing may be distinguished from breathing through the mouth based on the detected pressure, since nasal breathing has an intraoral pressure signature that is distinct from mouth breathing (e.g., as illustrated and discussed in FIGS. 10A, 10B, and 11 and the associated text). Pauses, delays, and interruptions of breathing may be identified. This data may be paired with other sensor data, including heartbeat/heart rate, head/body movement (e.g., from accelerometer data), etc. The apparatus may trigger one or more alerts or alarms based on respiration.

The methods and apparatuses described herein may be configured to monitor sleep, including detecting sleep apnea, snoring, and/or other sleep-disordered breathing. As mentioned above, the respiration may be monitored, and the resulting pressure data may be analyzed to identify sleep apnea, snoring, and the like. In some examples, the dental appliance is a mouthpiece (mouthguard, mask, etc.) for a sleep apnea treatment device, such as a continuous positive airway pressure (CPAP) machine that includes a sealed pressure sensor module as described herein. The operation of the machine may be regulated by the sensed pressure data. For example, if snoring and/or apneic events are detected the apparatus may trigger activation of positive pressure.

The methods and apparatuses described herein may be configured to monitor a subject's general health, including fitness. For example, any of these apparatuses may be used to monitor activity, including exercise tracking, e.g., by tracking respiration during exercise and/or everyday activity. Respiration rate may be used to calculate a level of effort exerted by the subject and/or a heart rate, as these metrics are correlated and can be derived (e.g., after calibration for the subject). In some examples, the determined respiration rate may be tracked along with other sensor data (e.g., movement data from an accelerometer, heart rate from a heart rate sensor, etc.). The respiration rate and any other suitable sensor data may be stored and/or analyzed. In some examples the pressure data may be used to determine an index of activity, e.g., based on respiration. An index of activity may provide a measure (absolute or scaled, e.g., on a 0-100 or 0-10 scale, et.) of activity based on respiration. For example, an aligner, retainer, mouthpiece, mouthguard, etc. configured as described herein may be worn during activity, including exercise, and may communicate either in real-time or following the activity (as in any of these apparatuses and methods, by transferring stored data to a remote processor) for analysis and correlation with activity monitoring. In some examples, the sensor may continuously/semi-continuously and/or periodically monitor a user's respiration including during a physical activity or exercise, and this information may be sent to a computing device (e.g., a mobile device, an exercise device) for display (e.g., as a raw number, as a visualization). The user may then be able to use this information to adjust exercise intensity so as to maintain a desired respiration rate/heart rate/effort level (e.g., to remain within a heart zone as required for a particular exercise program). In some examples, monitoring respiration rate/heart rate/effort may also be useful for patients who are at risk of overexertion (e.g., patients with heart failure, patients with pacemakers, patients with a respiratory illness, patients recovering from an illness or surgery),

The methods and apparatuses described herein may also be used to diagnose, treat, and/or monitor a condition, illness, or disorder, not limited to dental/orthodontic disorders. For example, the methods and apparatuses described herein may be configured to detect or monitor bruxism (e.g., tooth grinding). Data from the pressure sensor modules described herein may be used to determine (e.g., to detect and/or to quantify) tooth grinding, as pressure is applied by the teeth against the apparatus during tooth grinding. The applied pressure may deform the body of the dental appliance, and this deformation may result in deformation of the deformable surface (which is typically more deformable than the adjacent region of the body), even where the deformable surface of the sealed pressure sensor module is positioned remotely from the occlusal surface of the dental appliance. Bruxism (tooth grinding) may be determined based on the frequency (typically a high-frequency event) and pressure displacement of the encapsulation cavity that may be characteristic as compared to other signals received by the encapsulated pressure sensors. As further explained and illustrated herein, the disclosed methods and apparatuses are both sensitive and discriminating enough that bruxism can even be distinguished from other states like teeth clenching based on the pattern of pressure changes. The processor of the apparatus, or a separate processor in communication therewith, may monitor the pressure data over time to detect likely bruxism events, and may track the frequency, duration, and time of day (e.g., night, day), patient state (e.g., awake, sleeping, etc.) for storage and/or display. In general, the apparatus and methods described herein may be used as part of a sleep study or ongoing sleep monitoring.

Any of the apparatuses described herein may be used for monitoring a dental treatment, including monitoring the status and/or activity of the dental appliance and/or the dentition of the subject wearing the dental appliance. For example, any of these methods and apparatuses may be configured to track or monitor palatal expansion. Palatal expansion involves expanding the palate of the patient by using a palatal expander to apply forces to gradually widen the upper jaw. Palatal expansion typically results in separation of the palate by breaking a midline joint or “suture” to widen the jaw. In some cases, the breaking of the subject's suture may be detected by a relatively rapid change in the pressure on the dental appliance. This pressure change can be detected by incorporating the pressure sensor module described herein (e.g., along the palatal region of a palatal expander). The pressure data may be compared to one or more thresholds to determine if or when the pressure sensed on the apparatus exceeds a predetermined suture break threshold, indicating cracking or breaking of the suture leading to expansion of the palate.

Alternatively or additionally, these methods and apparatuses may determine, based on the sensed pressure, when the force being applied by the dental appliance (e.g., a palatal expander, an aligner) to the patient's dentition falls below a threshold indicating that he appliance is no longer applying sufficient force to effect the desired change in the dentition and the (e.g., a change appliance threshold). The pressure sensor module may be configured to detect pressure applied between the dental appliance and the dentition, including between the dental appliance and the patient's teeth and/or palate. The pressure may be transduced through the body of the dental appliance to the deformable surface of the airtight encapsulated cavity. As long as the apparatus is applying force to drive movement of the dentition, the apparatus may detect a pressure (or pressure offset) on the encapsulation cavity. If the pressure dips below the threshold, indicating that the applied force has decreased to an unacceptable level, the apparatus, or a processor in communication with the apparatus, may indicate that the dental appliance should be changed and replaced with a new dental appliance, including a next stage in a treatment plan. For example, a notification may be sent to a patient via a mobile app or via a communication (e.g., text message, email), recommending that the patient change the dental appliance. Additionally or alternatively, such notification may be sent to a dentist or other healthcare professional/provider. In some examples, the pressure may be tracked and stored on an application for analysis/monitoring. For example, during the course of an orthodontic treatment, the pressure may be monitored to determine the effectiveness of the treatment (e.g., during various stages of the treatment). Thus, the methods and apparatuses described herein may be configured to determine when to replace the dental appliance.

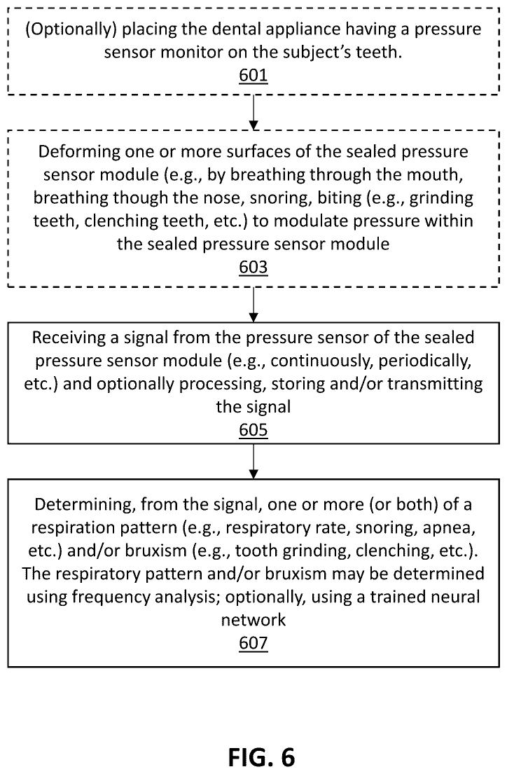

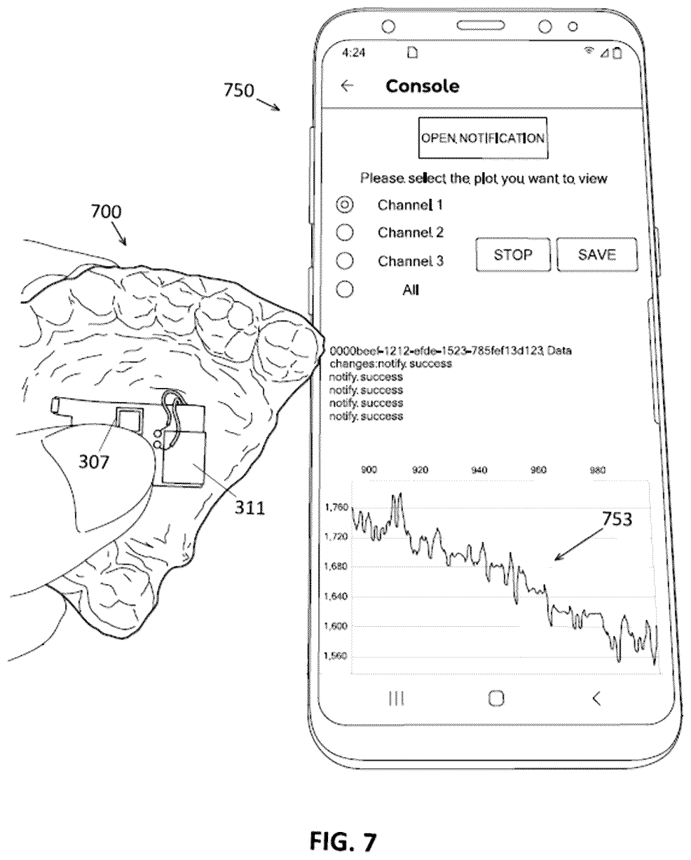

FIG. 6 illustrates one example of a method of using an apparatus as described herein. In FIG. 6, the method may optionally include placing a dental appliance having a pressure sensor monitor on the subject's teeth (e.g., over the teeth) 601. In any of these examples, the method may optionally include deforming one or more surfaces of the sealed pressure sensor module (e.g., by breathing through the mouth, breathing though the nose, snoring, biting, grinding teeth, clenching teeth, etc.) to modulate pressure within the sealed pressure sensor module 603, while the user is wearing the apparatus including the sealed pressure sensor module. Any of these methods may include receiving a signal from the pressure sensor of the sealed pressure sensor module (e.g., continuously, periodically, etc.) and optionally processing, storing and/or transmitting the signal 605. As mentioned, the pressure sensor signals may be sampled continuously or discretely (e.g., at a frequency, e.g., of between about 0.001 Hz to about 1 kHz). The pressure signals may be filtered, normalized, digitized, etc. and may be immediately analyzed (using a running window, or ongoing analysis) and/or may be stored for later analysis. The methods may further include determining, from the signal, one or more (or both) of a respiration pattern (e.g., respiratory rate, snoring, apnea, etc.) and/or bruxism (e.g., tooth grinding, clenching, etc.). The respiratory pattern and/or bruxism may be determined using frequency analysis; optionally, using a trained neural network 607.

### EXAMPLES

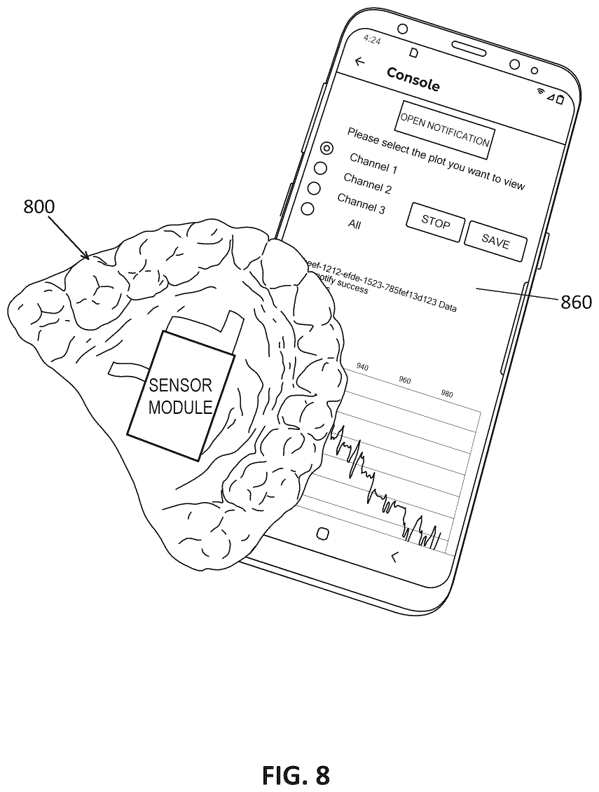

The dental appliances described herein including a sealed pressure sensor module may robustly function in virtually any oral environment for extended periods of time without significant degradation of the signal quality. The dental appliances including fully-sealed pressure sensor modules may directly or indirectly sense mechanical forces that may be applied on the dental appliance which may result in change in the deformable surface(s) of the sealed pressure sensor module, which may result in changes in the internal pressure of the sealed cavity of the sealed pressure sensor module. Pressure is the result of force (e.g., mechanical force) distributed over an area (e.g., the area of the deformable membrane). Thus, these apparatuses may sense external pressure(s) (e.g., environmental pressure within the oral cavity; mechanical forces from grinding, clenching, etc.). FIGS. 7 and 8 illustrate tests showing detection of direct mechanical force on the apparatus 700 including the dental appliance including a sealed pressure sensor module (FIG. 7) and indirect or external pressure on the dental appliance including a sealed pressure sensor module. The sensed pressure data may be transmitted, stored, and/or output as a visualization 753 on a user interface of a computing device 750 (e.g., a tablet, phone, laptop, etc.). In FIG. 8 an external pressure is applied by placing the apparatus 800 in a sealed bag (test chamber 860) and applying a mechanical force without any mechanism to vent to atmosphere, showing the sensitivity of the apparatus to air pressure.

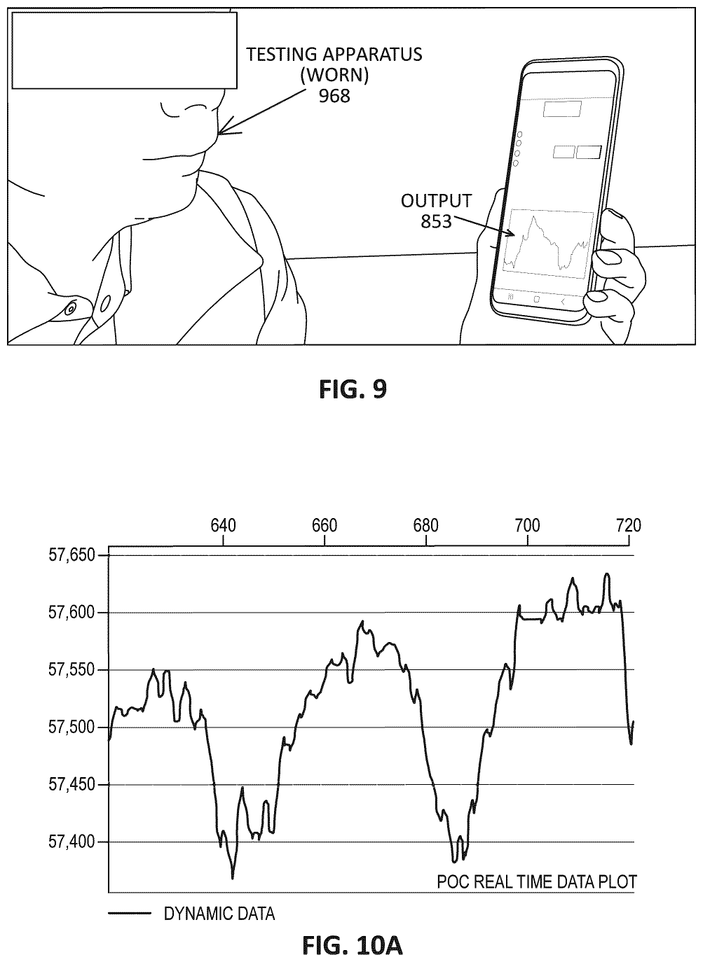

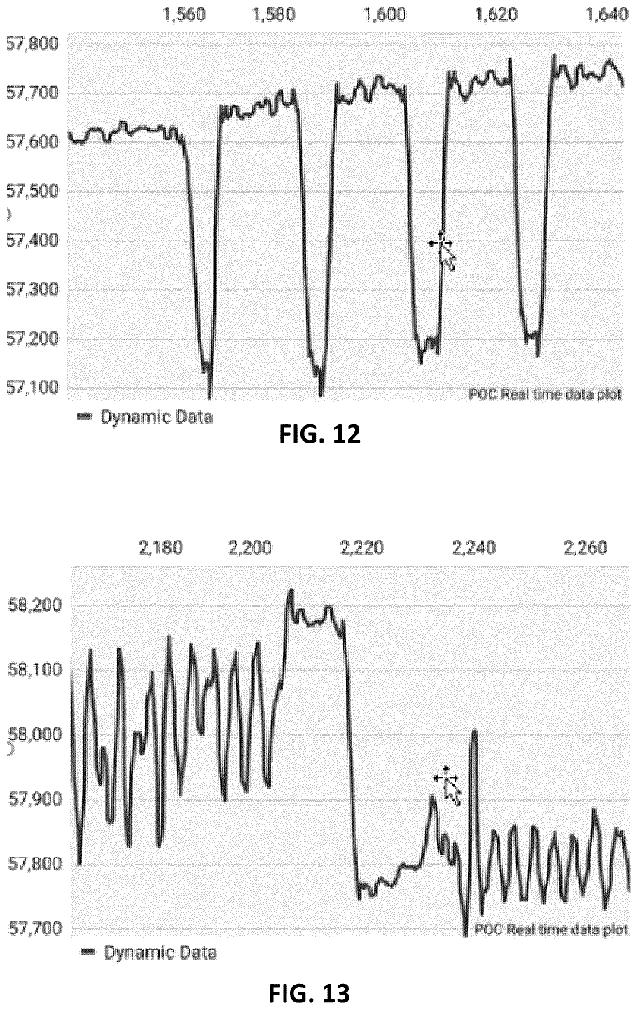

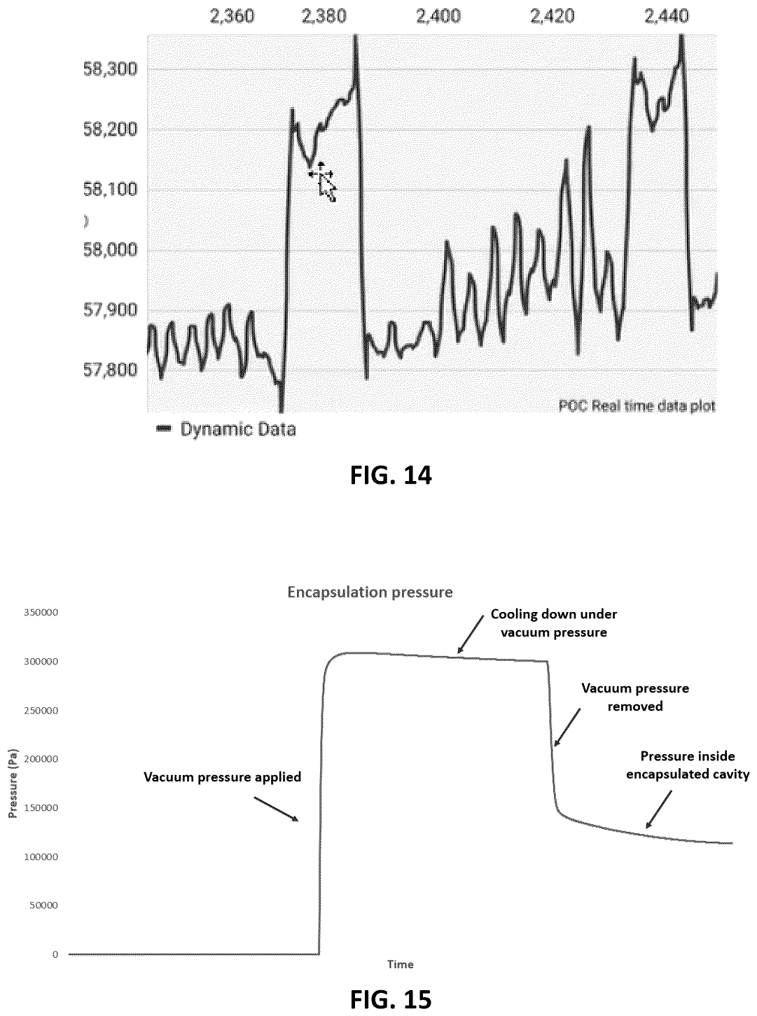

As shown in FIG. 9, an apparatus 968 similar to that shown in FIGS. 7 and 8 (and FIGS. 3 and 4A-4C) was examined for use within an oral cavity of a test subject, and the output 853 examined. The pressure output from the pressure sensor module of the dental appliance worn was correlated with different evoking conditions, including nose breathing (FIGS. 10A-10B), mouth breathing (FIG. 11), teeth clenching (FIG. 12), teeth grinding (FIG. 13), and teeth clenching and grinding (FIG. 14). As illustrated, distinctive characteristic pressure patterns were seen and can be readily resolved both manually and automatically, even when using the same dental appliance. Further distinguishing features may be identified when changing the relative location and/or orientation of the pressure sensor module relative to the dental appliance body.

In the data shown in FIGS. 10A-14, the sensed pressure signals were detected from a palatal area, as the scaled pressure sensor module was positioned on the upper portion of the palatal region of the device. Thus, the apparatus was particularly sensitive to changes in air pressure, which were surprisingly found to be characteristic for different types of respiration and jaw activity, resulting in significant changes in pressure within the encapsulated cavity (in this example, a sealed air-filled cavity). Even relatively small deformations of the deformable surface of the encapsulated cavity caused sufficient pressure changes so as to be detected by the disclosed pressure sensor module. In FIGS. 10A-14, the y-axis shows relative pressure, and x-axis shows time (the units are not calibrated in these examples).

For example, in FIGS. 10A-10B nose breathing resulted in a distinct pattern of flattened peaks and troughs reflecting transitions between inhalation and exhalation as air is entrained from the nasal region to the oral cavity. In contrast, FIG. 11 shows the pressure pattern characteristic of respiration through the oral cavity, with the mouth open. This pattern has a more gradual increase and decrease between inhalation and exhalation.

Distinct patterns for intentional tooth clenching were also seen in FIG. 12, showing a change in pressure resulting from deformation of the body of the dental appliance, resulting in deformation of the attached deformable surface of the encapsulated cavity. Tooth grinding resulted in a different characteristic pattern, as shown in FIG. 13, showing relatively higher frequency pressure waves having a slightly lower amplitude as compared to tooth clenching. A combination of grinding and clenching resulted in a pressure pattern including the combination of characteristic patterns of both FIGS. 12 and 13, as shown in FIG. 14, showing both the smaller amplitude, higher frequency tooth grinding pressure pattern acting on the body of the dental appliance and the larger amplitude, lower frequency tooth clenching pressure pattern.

In general, these apparatuses, and in particular the sealed pressure sensing modules described herein may have excellent moisture resistance, with no visible water ingress and full functionality maintained during and after extended exposure to a wet or moist environment. For example, initial testing with an apparatus such as that shown in FIGS. 3 and 4A-4C above found that even after submersion of a sample in water for ˜5.5 hours or longer, there was no detectable change in pressure sensitivity or the ability of the apparatus to detect changes in pressure on the dental appliance.

All publications and patent applications mentioned in this specification are herein incorporated by reference in their entirety to the same extent as if each individual publication or patent application was specifically and individually indicated to be incorporated by reference. Furthermore, it should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

Any of the methods (including user interfaces) described herein may be implemented as software, hardware or firmware, and may be described as a non-transitory computer-readable storage medium storing a set of instructions capable of being executed by a processor (e.g., computer, tablet, smartphone, etc.), that when executed by the processor causes the processor to control perform any of the steps, including but not limited to: displaying, communicating with the user, analyzing, modifying parameters (including timing, frequency, intensity, etc.), determining, alerting, or the like. For example, any of the methods described herein may be performed, at least in part, by an apparatus including one or more processors having a memory storing a non-transitory computer-readable storage medium storing a set of instructions for the processes(s) of the method.

### Baseline Pressure Within the Airtight Encapsulated Cavity

In any of the apparatuses and methods described herein, the baseline pressure within the airtight encapsulated cavity of the sealed pressure sensor module may be pre-set to increase the sensitivity of the pressure sensor modules described herein, including tuning the pressure sensor module sensitivity. In general, this pressure may be greater than the surrounding atmospheric pressure (e.g., greater than 1 atmosphere, greater than 1.1 atmosphere). In some examples the pressure may be approximately 1 atmosphere.

In some examples, when forming the sealed pressure sensor modules described herein, after or during encapsulation of the pressure sensor within the airtight encapsulated cavity, the cavity may be pressurized. For example, during the thermoforming process, the multi-sensor unit may be sealed in a pressure cavity so that the pressure with the sealed cavity may be at or greater than the external atmosphere pressure (e.g., approximately 1 atm, 1.1 atm, 1.2 am, 1.3 atm, 1.4 atm, 1.5 atm, 1.6 atm, 1.7 atm, 1.8 atm, 1.9 atm, 2.0 atm, etc.). FIG. 15 illustrates an example in which the pressure within the airtight encapsulated cavity of the pressure sensor module is encapsulated through a thermoforming process. During one example of a thermoforming process, an elastically deformable material layer (e.g., a polymer) is arranged over a mold in a thermoforming chamber, and the electronics of the pressure sensor module may be positioned over the elastically deformable material layer. An adhesive or coating may be optionally added as explained previously, e.g., with respect to FIGS. 2A and 2B. A plastic layer may be heated and disposed over this assembly (e.g., over the mold, the elastically deformable material layer, and the electronics). A vacuum pressure may then be applied inside the thermoforming chamber to force the plastic layer to fully cover the mold. Due to this applied vacuum pressure, the trapped air between the base and top layer is squeezed in the region forming the airtight encapsulated cavity. As illustrated in FIG. 15, sensor measurements taken by the encapsulated sensor electronics show the resulting sharp pressure increase (e.g., causing pressure to reach ˜300,000 Pa in this example) within the formed airtight encapsulated cavity. The thermoformed device, including the combination of the appliance and the pressure sensor module, may then be cooled, which results in a slow drop in pressure within the airtight encapsulated cavity, as shown in FIG. 15. Once the apparatus has cooled to a sufficient temperature, vacuum pressure in the thermoforming chamber is removed, resulting in a sudden drop in internal pressure measured by the pressure sensing module, as shown in FIG. 15. The apparatus continues cooling until it achieves its final state, as can be seen by a final gradual decrease in pressure in FIG. 15. At this point, the internal pressure has stabilized to a desired level (e.g., in this example, around 120,000 Pa, approximately 1.2 atm), and the pressure sensor is outputting the cavity's internal pressure. Other methods for forming the pressure sensor modules, including pressuring the airtight encapsulation cavities, may be used, including forming the pressure sensor module separately from the device to which it is coupled, and/or encapsulating the cavity containing the pressure sensor by a bonding process done in a high pressure chamber, etc.

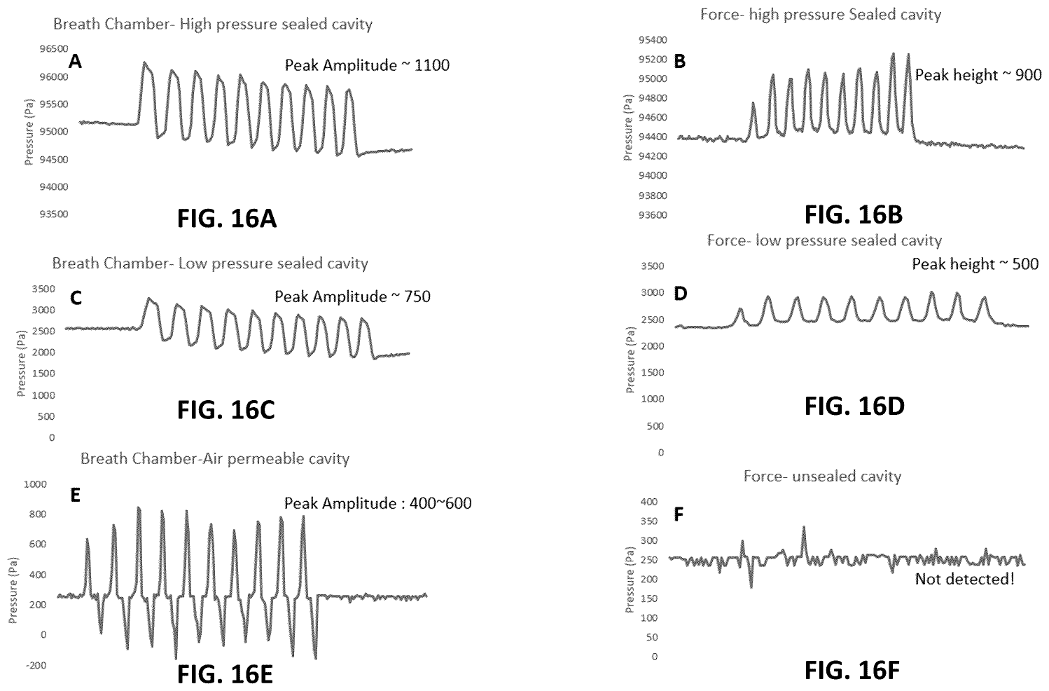

Sensor measurements of internal pressure of the airtight encapsulated cavity may now be used to derive external pressure (e.g., from oral cavity air pressure and/or mechanical forces). Referring to equation 1, sensed external pressure may be based on the amount of pressure change (AP) inside the cavity, which can be derived when the baseline pressure and the internal pressure (as measured by the pressure sensor module) is known. In some examples, the calculation of the external pressure may be further based on the material properties of the elastically deformable surface and the plastic material forming the appliance, such as the stiffness of these layers. In some examples, the calculation of the external pressure may be further based on the cavity baseline pressure as will be explained in further detail below in association with FIGS. 16A-16F. In some examples, the cavity may be pressurized to a relatively high pressure (e.g., as compared to ambient pressure). Creating a pressure cavity (in some examples, a high-pressure cavity) during encapsulation as shown in FIG. 15 (which could be, e.g., in the range of 90,000˜150,000 Pa, e.g., 0.9 atm to about 1.5 atm, or more) may help amplify the AP caused by changes in oral cavity pressure or applied force (e.g., teeth grinding, clenching, breathing, and etc.). In particular, it may be beneficial to use a pressure that is higher than one atmosphere (e.g., 1.1 atm or greater, 1.2 atm or greater, etc.) and/or higher than oral cavity pressure within the airtight encapsulated cavity.

For example, FIGS. 16A-16F illustrate comparisons between different cavity pressures, showing the effect of the cavity pressure in a sealed (FIGS. 16A-16D) versus air-permeable encapsulation (e.g., unsealed, FIGS. 16E and 16F). In these examples, an appliance that included a pressure sensing module was subjected to conditions simulating a subject wearing the appliance, and measurements were taken while simulating breathing or teeth grinding/clenching. In this example, FIGS. 16A and 16B illustrate measurements taken from a first dental appliance having a pressure sensing module including an airtight encapsulated cavity with a relatively high pressure. Pressure was measured during simulated breathing (FIG. 16A) and simulated grinding/clenching (by applied mechanical forces, FIG. 16B). FIGS. 16C and 16D illustrate pressure measurements taken from a second dental appliance having a pressure sensing module including an airtight encapsulated cavity with a relatively low pressure. FIG. 16C shows the pressures measured during simulated breathing and FIG. 16D shows the pressures measured during simulated grinding/clenching. Finally, FIGS. 16E and 16F illustrate examples in which the cavity is not sealed at all, but is an air-permeable cavity partially enclosing the one or more pressure sensor(s). FIG. 16E shows pressures measured during simulated breathing and FIG. 16F shows pressures measured during simulated grinding/clenching. The traces shown in FIGS. 16A-16F therefore illustrate examples of the output of pressure sensor modules having cavities with different internal pressure conditions, under different physiological conditions (respiration or tooth grinding).

As shown in FIGS. 16A-16F, the pressure sensor module having a high-pressure sealed cavity (FIGS. 16A and 16B) had the highest sensitivity when detecting external pressure (both oral cavity pressure during breathing and mechanical force). The low-pressure sealed cavity detected pressure changes during breathing (see FIG. 16C) at lower sensitivity than the high-pressure cavity sensor module (FIG. 16A). Similarly, the pressure sensor module having an air-permeable (unpressurized) cavity sensed pressure changes (with relatively low sensitivity) and were not able to detect sustained pressure differences. The pressure sensor module having a low pressure sealed cavity detected the simulated tooth grinding/clenching (FIG. 16D) with lower sensitivity than the pressure sensor module having a high pressure sealed cavity (FIG. 16B). In contrast, the pressure sensor module having an air-permeable (unpressurized) cavity was unable to resolve applied force at all, as it returned only noise (FIG. 16F). Thus, simulated tooth grinding/clenching was readily detected when the pressure sensor was encapsulated in a fully air-sealed cavity and the amplitude of sensed pressure was amplified by increasing the internal cavity pressure within the airtight encapsulated cavity. Increasing the cavity pressure (e.g., >1 atm) may result in a greater sensitivity at larger internal pressures within the airtight encapsulated cavity.

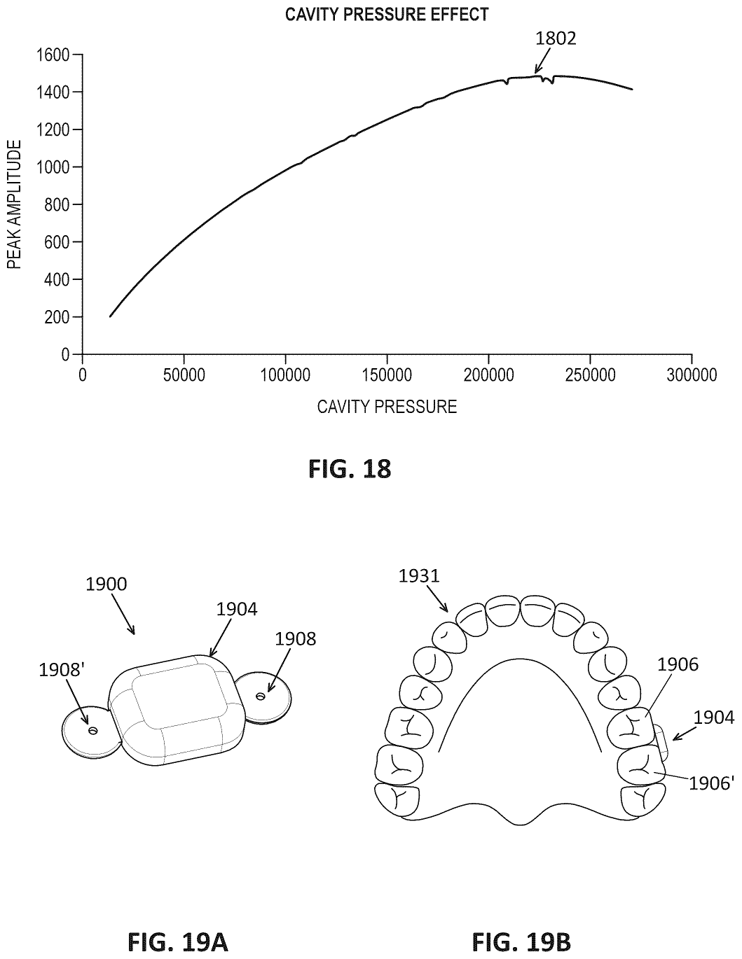

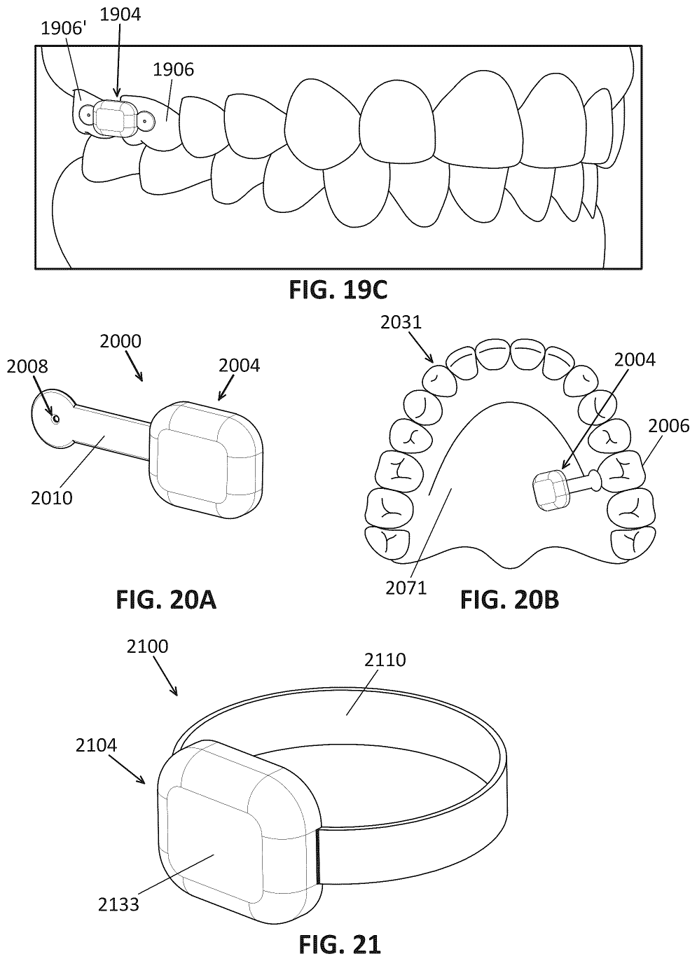

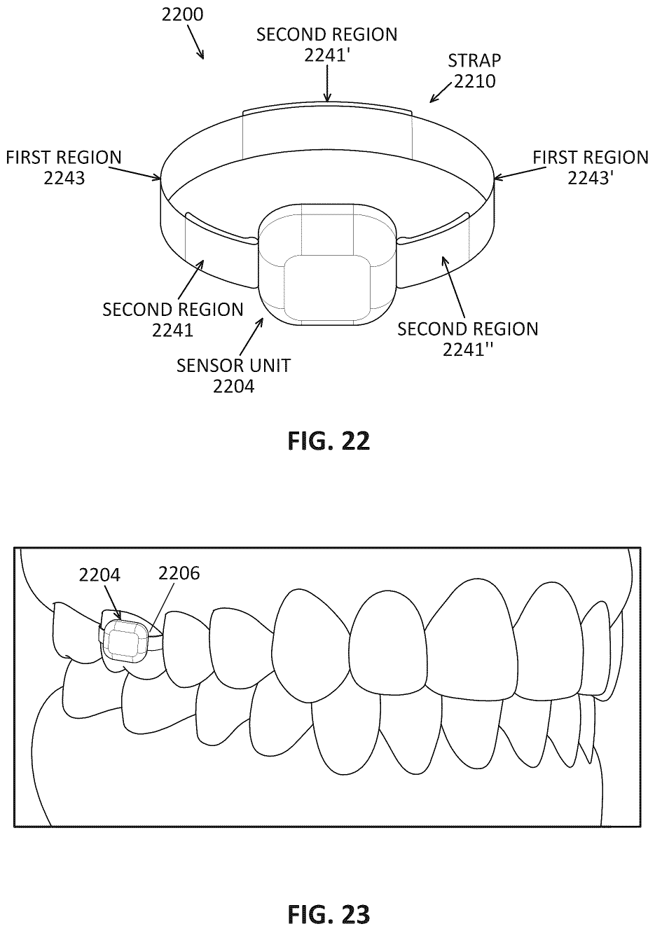

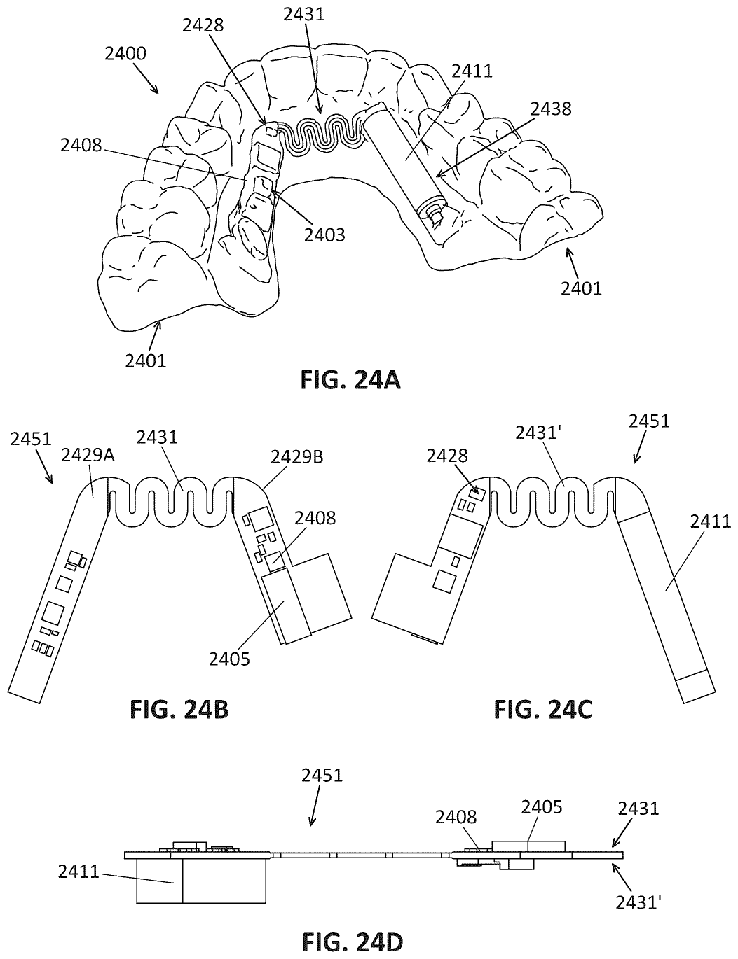

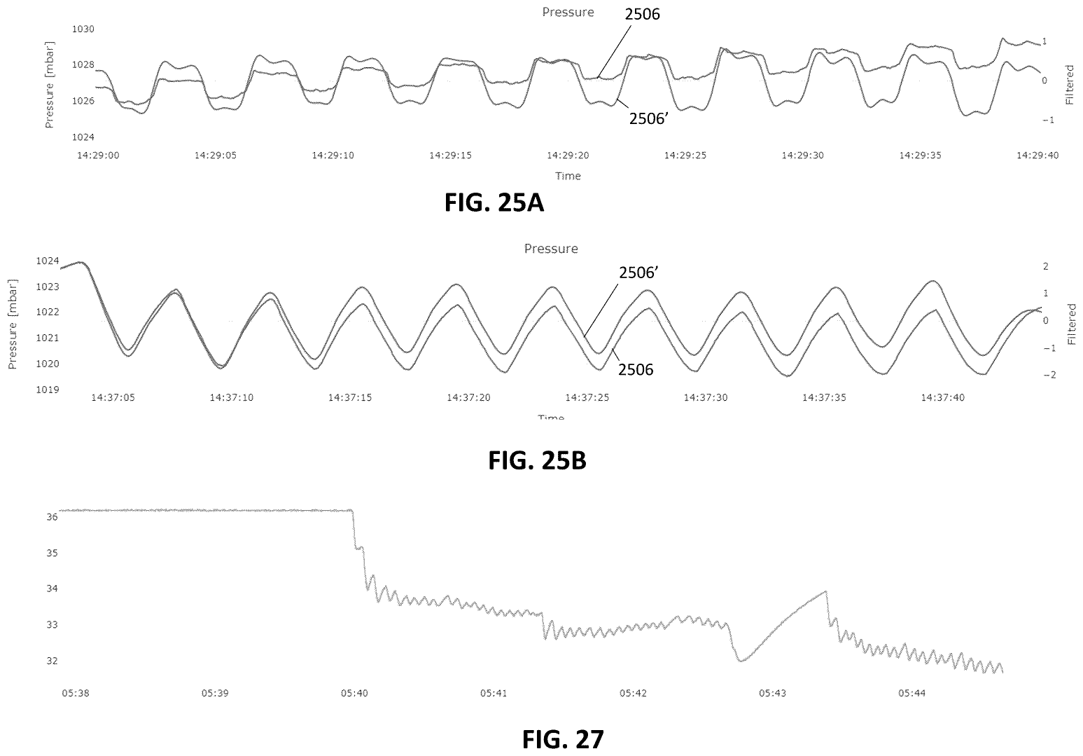

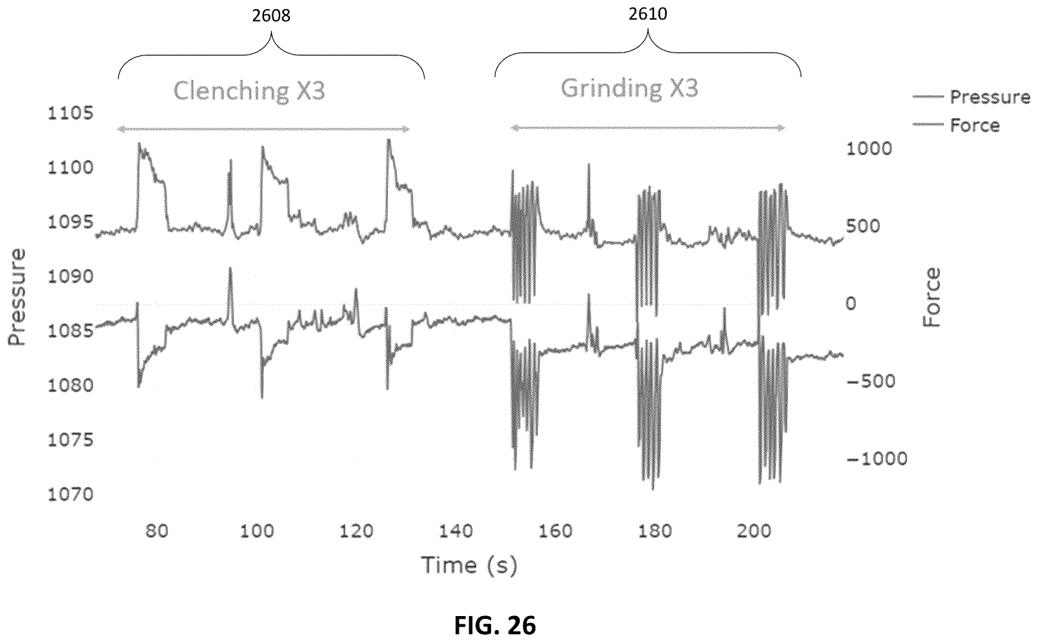

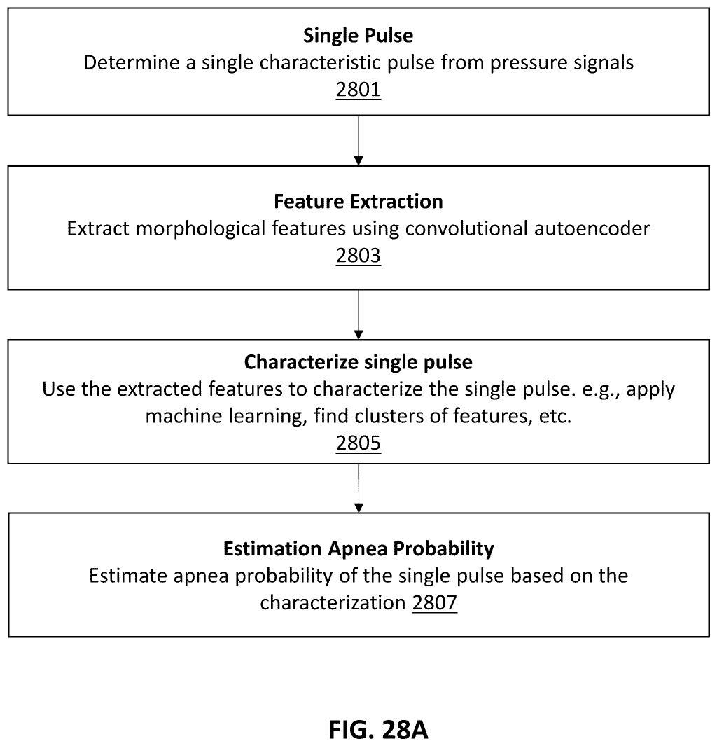

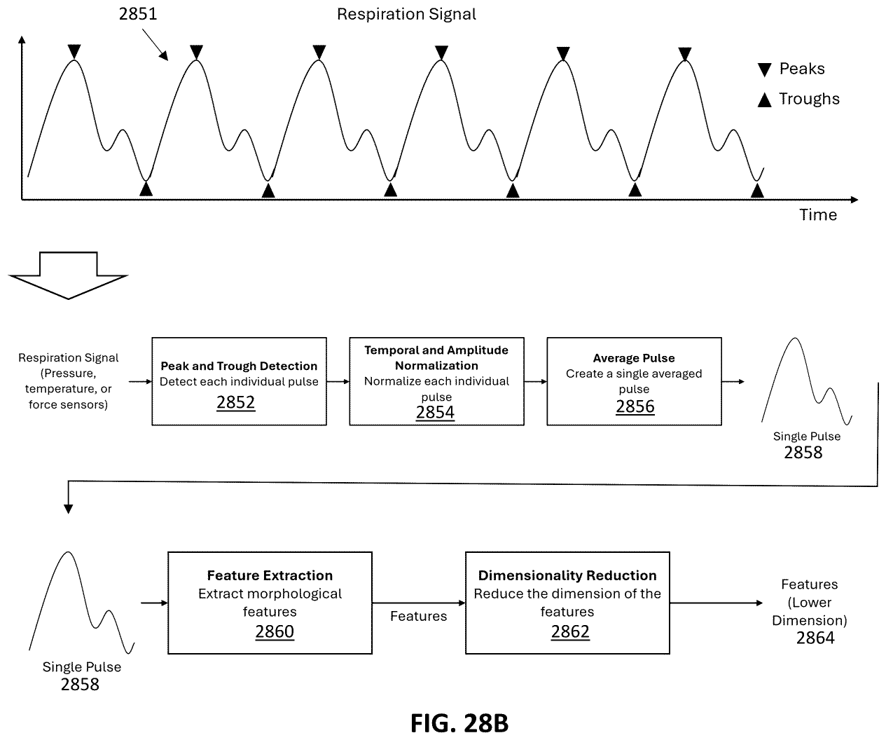

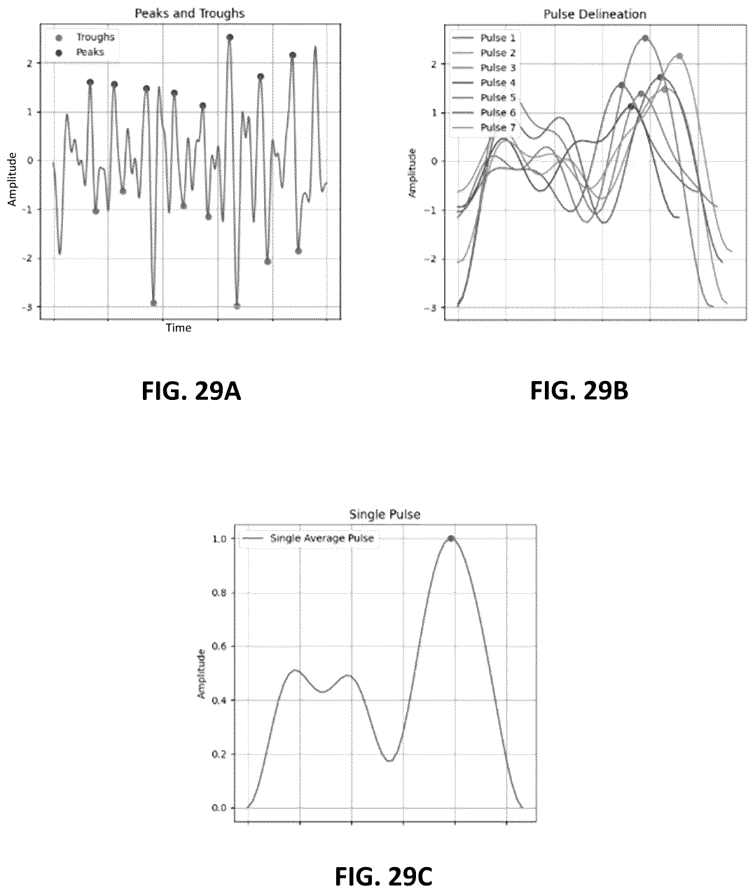

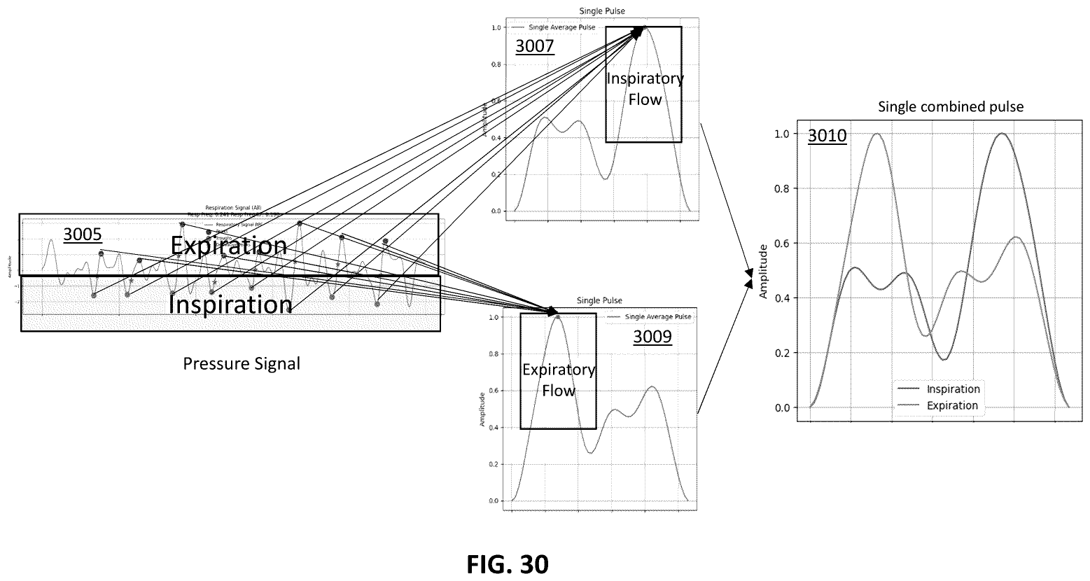

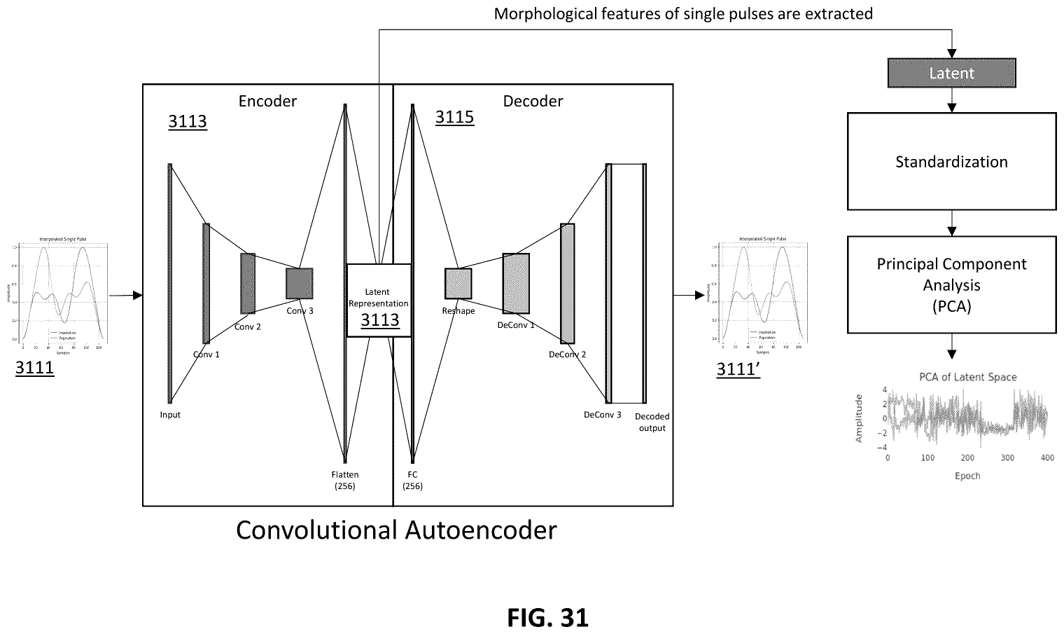

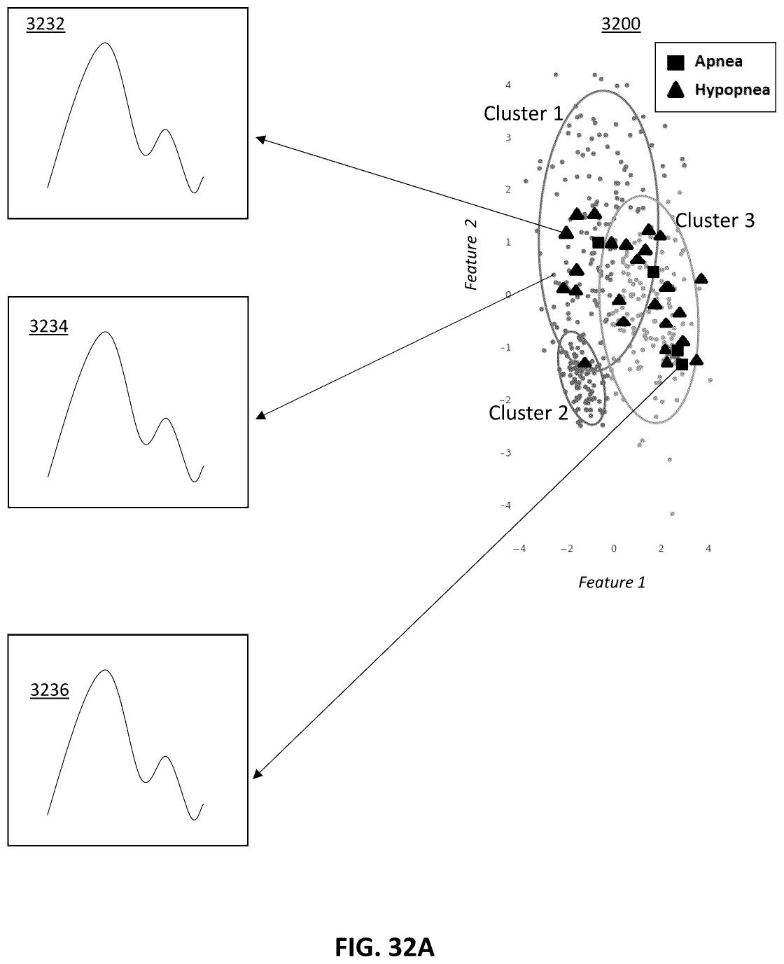

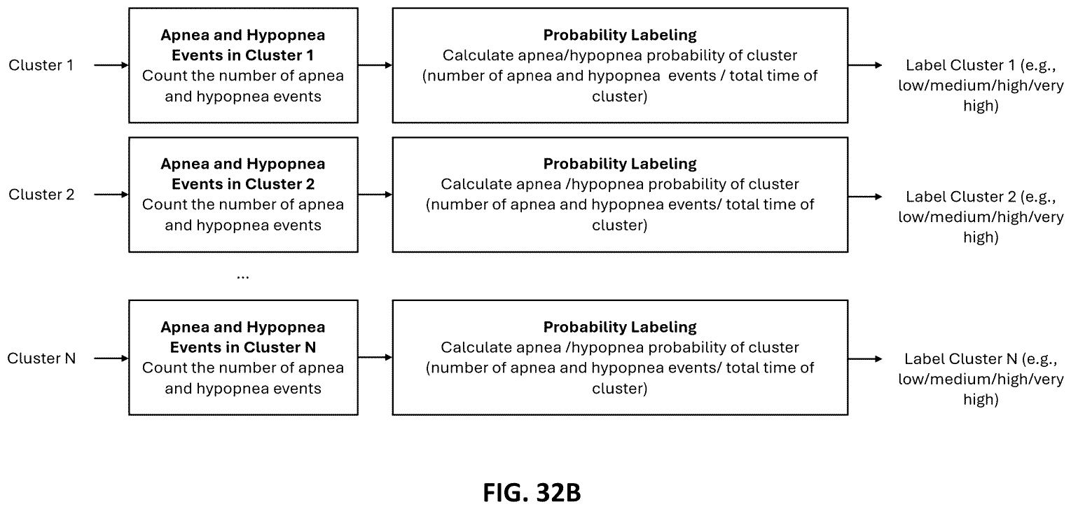

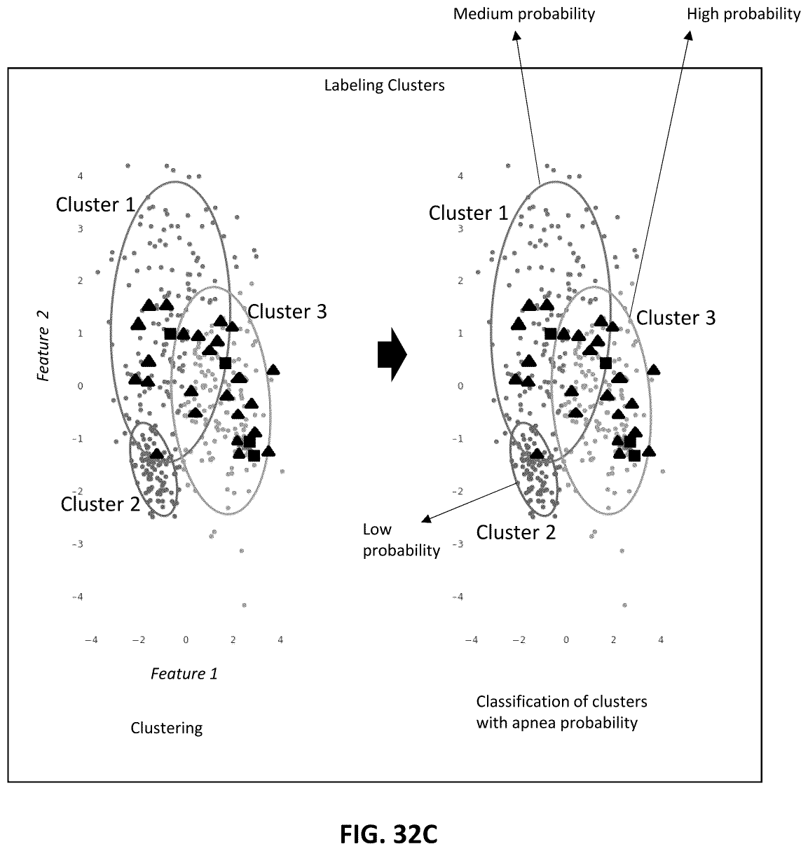

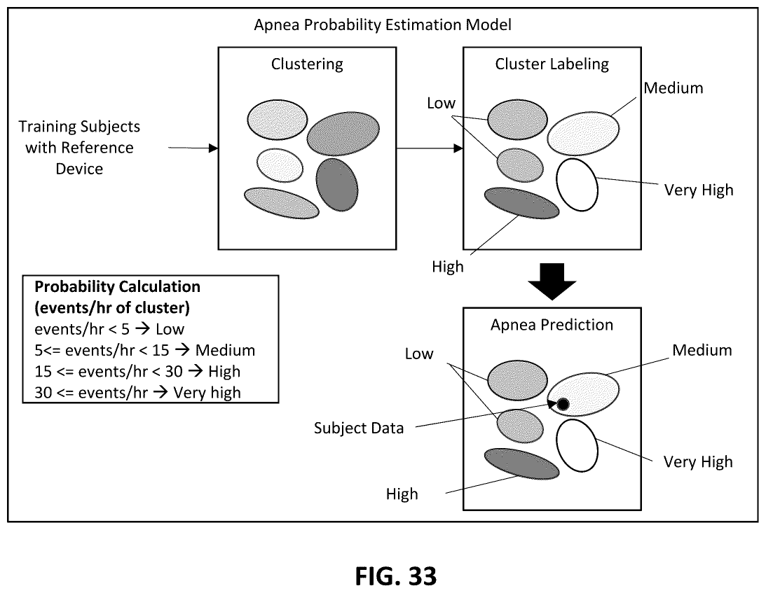

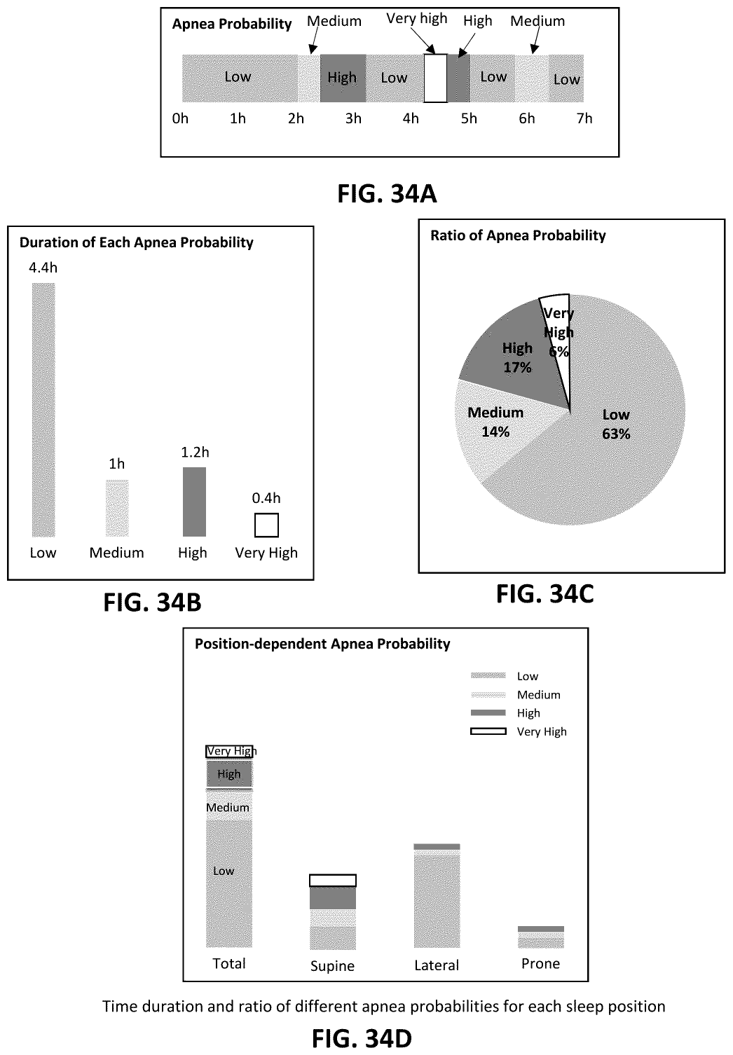

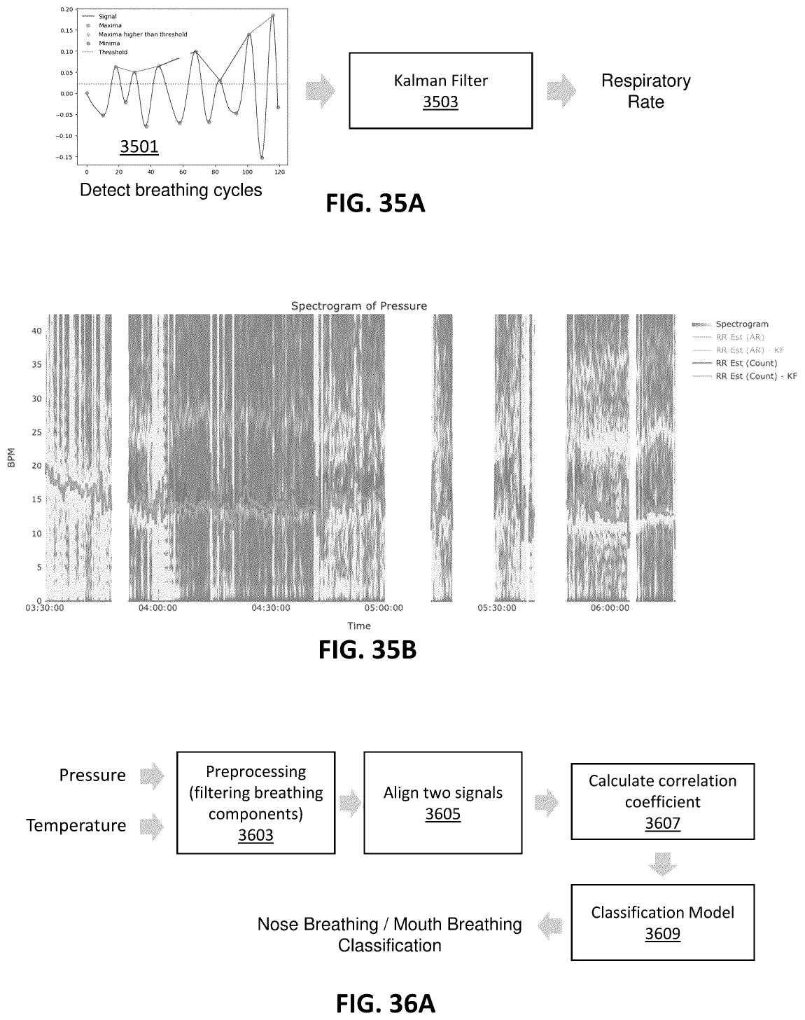

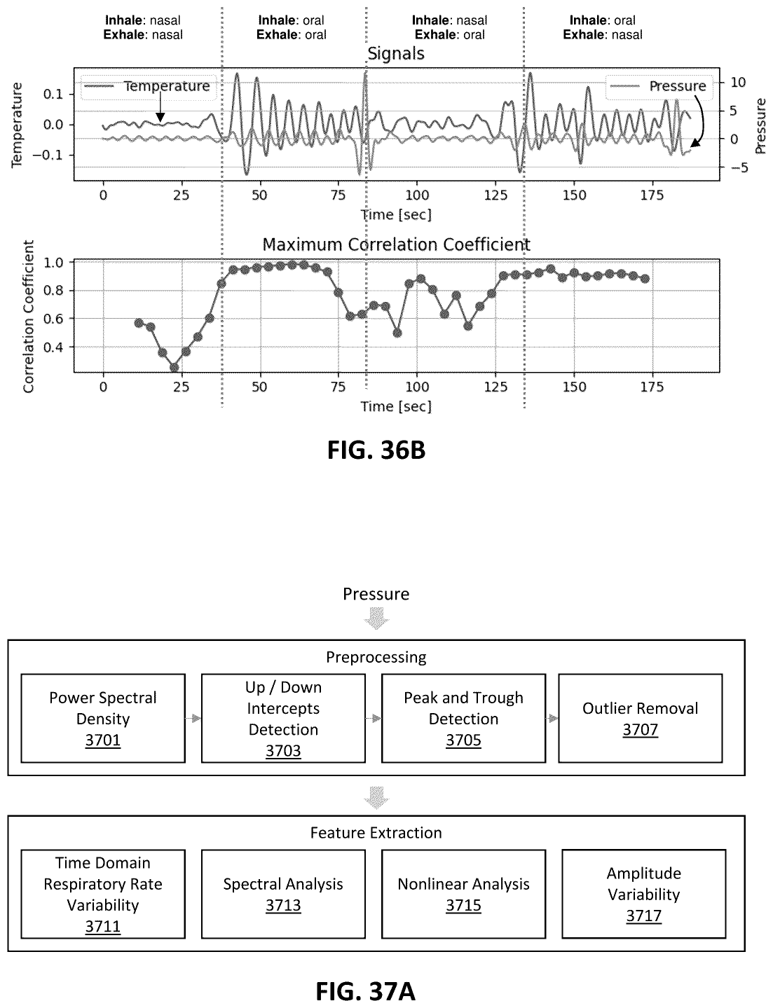

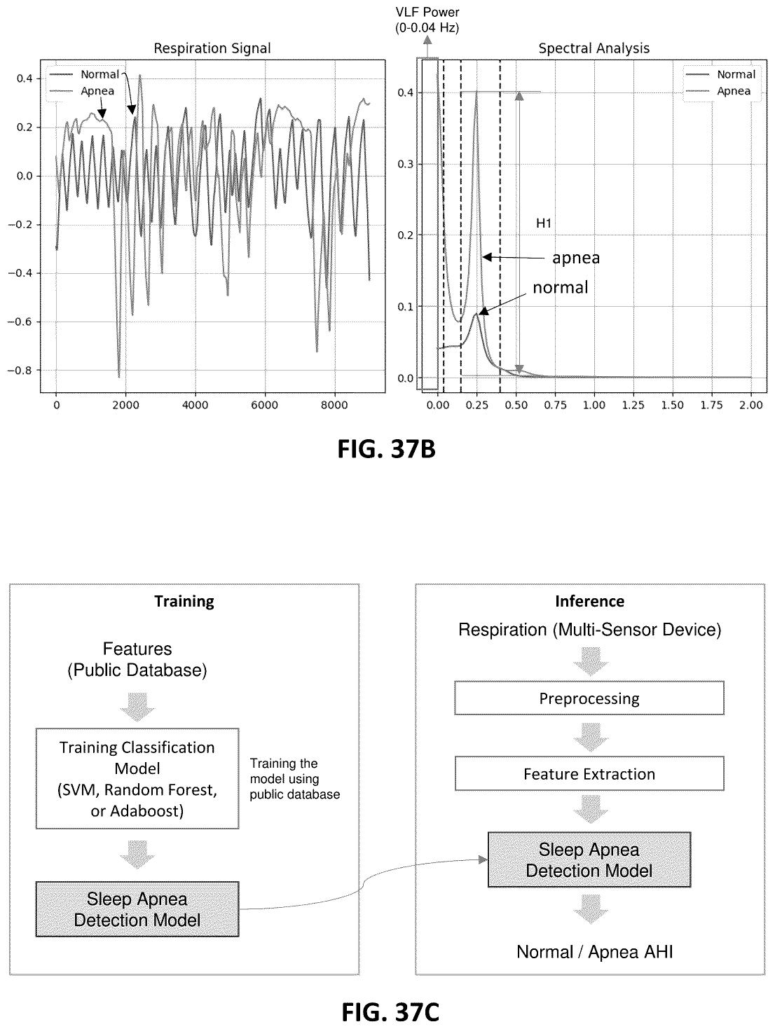

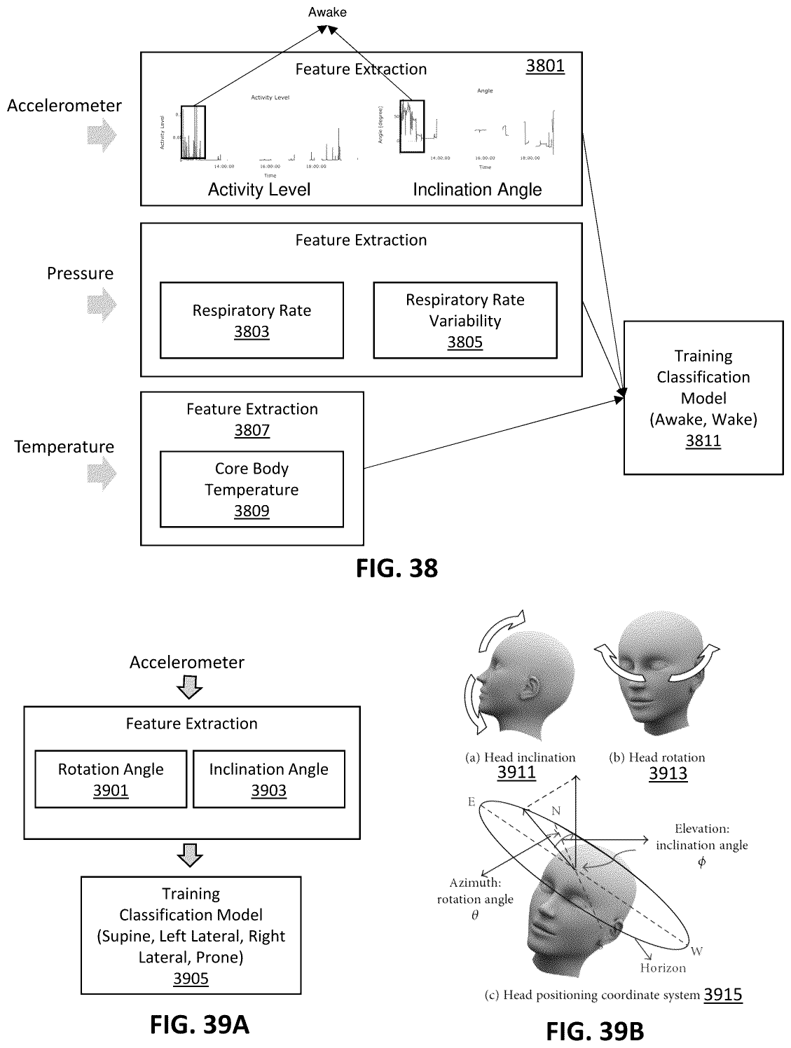

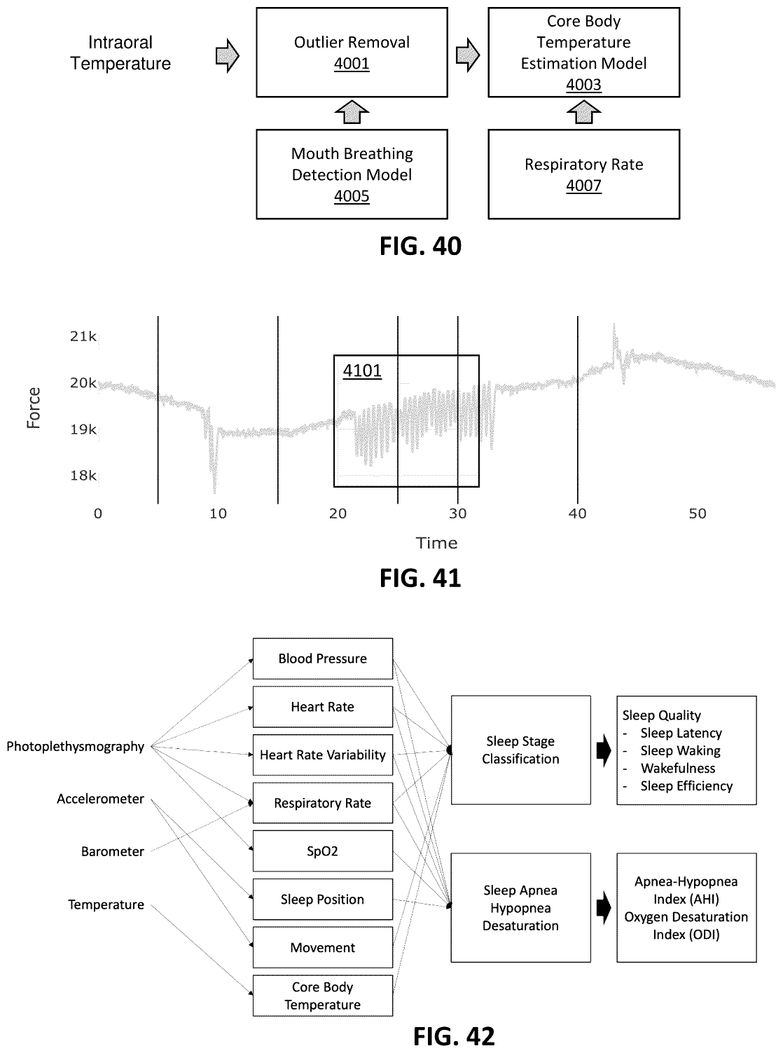

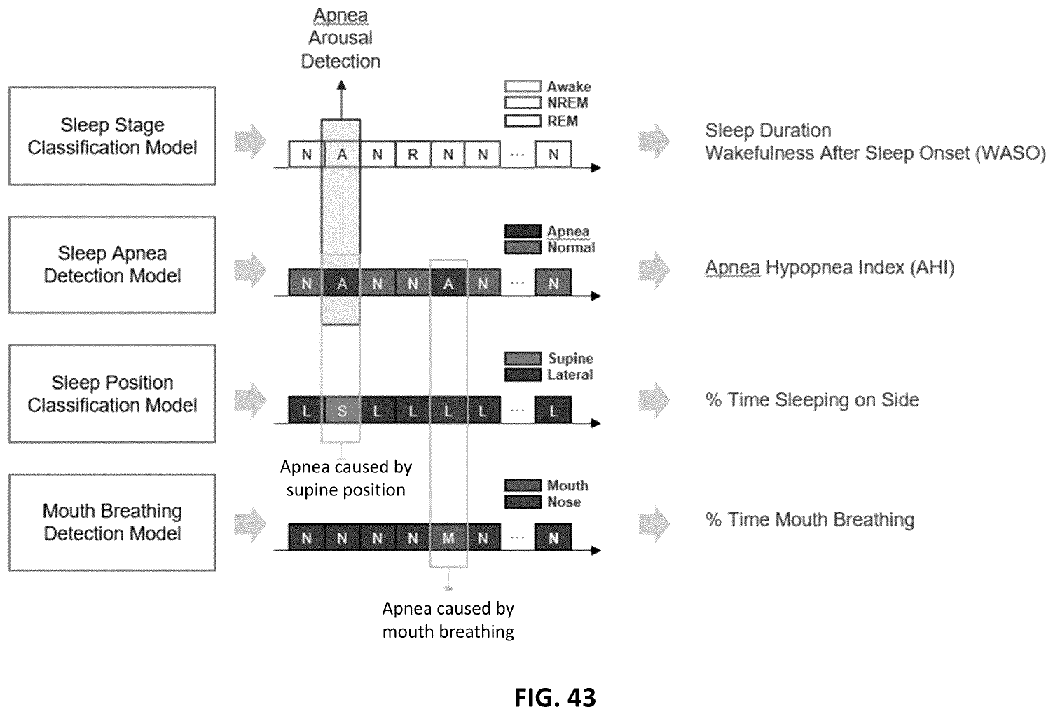

This effect was also seen when modeling of one example of a sealed pressure sensor module, as shown in FIGS. 17A-17B and 18. The 3D model shown in FIGS. 17A-17B was created in a COMSOL Multiphysics. In this model, an air cavity is created between two deformable layers (with approximately the same material properties for the base 1739 and the top 1733 layer) and a solid body 1705 (representing the pressure sensor encapsulated within the airtight encapsulated cavity, which may include one or more sensors (e.g., a multi-sensor unit) enclosed inside the cavity. In this model a point force is applied to the base layer and the pressure change inside the cavity (AP) is recorded under different initial cavity pressures. The pressure peak amplitude (y-axis) versus baseline pressure (x-axis) plot is provided in FIG. 18. In FIG. 18, the graph shows the change in cavity pressure peak amplitude (e.g., change in pressure, e.g., in Pa) as the cavity pressure increase. As shown in FIG. 18, increasing the cavity pressure may increase the sensor output or cavity pressure change (AP. However, after some point (maximal value 1802) the increase in AP is at a maximum and may decline. On the other hand, this internal pressure may be limited to how much the top and base material and laminated binding between them can tolerate, especially when external force and pressure is applied. For example, in some cases the pressure may be limited be greater than 1 atmosphere but less than 2.5 atmospheres (e.g., between 1.1 atmosphere and 2.4 atmospheres, between 1.2 atmospheres and 2.4 atmospheres, etc.). In some examples, the pressure within the airtight cavity may be higher than 170,000 Pa (about 1.6 atm) before the material of the deformable surface or the bonding breaks in a way that there is no longer airtight seal between the internal cavity and the external environment. In some examples, the encapsulated cavities may be pressurized to an optimal pressure value that is determined based on a pressure simulation or function (e.g., the maximal value 1802 in FIG. 18). In some examples, the optimal pressure value is further based on the material qualities of the deformable surface (e.g., by reducing the pressure value below an upper limit for the material). The airtight encapsulated cavity may be pressurized to a pressure that is equal to or slightly less than (e.g., 5%, 10%, 15%, 20%, 25%, 30%, 35%, etc. less than) the pressure the peak cavity pressure effect 1802. In some cases it may be beneficial to use the peak or slightly lower values, which may reduce strain on sealed pressure sensor.

As mentioned above, the airtight encapsulated cavities of any of these apparatuses may be filled with a compressible fluid (e.g., air) or a substantially incompressible fluid (e.g., water). In some examples it may be desirable to use a substantially incompressible fluid within the airtight encapsulated cavity, which may result in a higher sensitivity.

The apparatuses and methods described herein may include any appropriate pressure sensor encapsulated within the airtight encapsulated cavity and configured to measure pressure within the airtight encapsulated cavity. For example the pressure sensor may be a barometric pressure sensor that detects atmospheric pressure. A typical example of a barometric pressure sensor is a piezo-resistive type that uses silicon semiconductor. Piezo-resistive pressure sensors may utilize, e.g., a single Si crystal plate as a diaphragm and diffuses impurities on its surface to form a resistive bridge circuit, making it possible to calculate pressure (atmospheric) by detecting the resistance change resulting from distortion of this resistive bridge when pressure is applied. In some examples, the pressure sensor is an acoustic sensor that is configured to measure a change in sound waves in the external environment (or intraoral space inside mouth).

Multiple pressure sensors may be included with any of these apparatuses and methods, including multiple pressure sensors within the within the airtight encapsulated cavity. In some examples, multiple different pressure sensors may be used, including one or more within the airtight encapsulated cavity and/or multiple different types outside of the airtight encapsulated cavity.

For example, the methods and apparatuses described herein may include a combination of sealed and open pressure sensor modules. The combination of a sealed pressure sensors (e.g., within the airtight encapsulated cavity) and opened pressure sensors (outside of the airtight encapsulated cavity) included as part of the same pressure sensor module may provide, ins some examples, an increase the sensitivity and/or an increase in the ability of the apparatus to distinguish between respiration and other phenomena (e.g., bruxism, clenching, etc.). Two simultaneous pressure sensors, one in a sealed cavity and one in an air-permeable (or open) cavity may detect different pressure phenomena. The pressure sensor in the airtight encapsulated cavity may detect both an applied force (e.g., tooth clenching) and an external pressure change (e.g., respiration) but the pressure sensor outside of the airtight encapsulated cavity (in an open configuration) may only detect the external pressure change (e.g., breathing), as described in FIG. 16. Thus, a differential analysis of these signals may be performed to provide enhanced detection of different phenomena. In some examples signal or peak amplitudes corresponding to the same breathing event or to external pressure changes from both sensors can be added to increase the sensitivity. Thus, comparing and/or combining the output signals from both sensors may help in interpreting data captured from the sensor in the airtight encapsulated cavity.

The apparatuses described herein including one or more pressure sensor modules may have a variety of different form factors, in addition to those shown in FIGS. 2A-2B, 3, 4A-4C and 7, discussed above. For example, the pressure sensor module may be part or may include a ring configure to couple to a tooth, an anchor and/or an attachment. FIGS. 19A-19C, 20A-20B, 21A-21B and 22 illustrate one example of an apparatus configured to attach to one or more tooth for detecting pressure as described herein.

In some examples the pressure sensing module is part of a device that includes a strap as part of a mount. The device can be a standalone device that can be clipped, snapped, placed around, or otherwise secured onto a structure (e.g., a tooth, a set of teeth, an implant, gingiva, tissue) inside the mouth. For example, a device including a pressure sensing module having a strap can be attached to a tooth (or multiple teeth) or placed and formed around a palatal area as shown in FIGS. 19A-19C. In this example, the device including a pressure sensing module 1900 can be removed when it needs to be washed, when teeth need to be brushed, or for any other purpose, but may otherwise remain securely attached to the teeth (or a tooth). As shown in FIG. 19A, the device 1900 includes an enclosed pressure sensing module 1904 and a pair of anchor attachments 1908, 1908′ on either side of the pressure sensing module. In one example the tooth or teeth to which the device is to be attached can be configured to receive the device. For example, the tooth 1906 and/or the tooth 1906′ can include one or more anchors (e.g., attachments) on the surface of the tooth (or teeth) so that the device may be easy to attach and remove the device with a mating feature, e.g., anchor attachments, through the anchors on the tooth surface.

In some embodiments, the device including a pressure sensing module may be configured to be secured to one or more molar teeth. In such devices, the strap may accordingly be dimensioned for molar teeth. Positioning the oral thermal sensing device on the molar teeth may be advantageous due to its location at the back of the mouth, closer to respiratory airways. Furthermore, the device including a pressure sensing module may be less intrusive during everyday wear and less visible when placed on the molar teeth.

FIGS. 20A-20B illustrate another example of a device including a pressure sensing module 2000 configured as a stand-alone device. In this example, the device also includes a pressure sensing module 2004 and an anchor attachment 2008. The anchor attachment may be similar to the anchor attachment in FIGS. 19A-19C including an engagement channel or opening into which an anchor may be removably inserted. In the example device shown in FIG. 20A the anchor attachment is coupled to a strap 2010 that is coupled to the pressure sensing module. In some examples the strap may be relatively stiff, to hold the pressure sensing module against or near the target region of the oral cavity when the anchor attachment is engaged with an anchor in the oral cavity, as shown in FIG. 20B. in this example the device is secured in position against the palatal region 2071. In other examples the strap can configured to be shapable or moldable by hand; thus, for each individual anatomy, the strap can be manually shaped to fit into the space inside the mouth that may allow it to fit better and be more comfortable to wear.

Also described herein are apparatuses in which the device including the pressure sensing module also includes an elastically adjustable strap to secure the attached sensor unit to one or more teeth. An example of this configuration is shown in FIG. 21. In this example the strap is configured to attach to one or more teeth (although FIG. 21 illustrates a strap around one tooth, the disclosure contemplates that a similar strap may be extended around multiple teeth, e.g., adjacent teeth). The strap is configured as a flexible ring or band, that can be wrapped around a tooth (or teeth) for secure placement of the device. As shown in FIG. 21, the device 2100 including the pressure sensing module also includes a strap 2110 that is configured to attach to a tooth (or in some examples two teeth, or more than two teeth). The strap is formed of an elastic material (e.g., a material that is elastically deformable) and the strap is elastically adjustable. This may include all or a portion of the strap. A pressure sensing module 2104 (which may include a pressure sensor within the airtight encapsulated cavity, power source, communication circuit, and one or more controllers, not shown) may be coupled to the strap and may include a smooth (atraumatic) outer surface 2133. In FIG. 21, a subject wearing the device including the pressure sensing module may apply the device by manually attaching it over the one or more teeth so that the deformable surface of the pressure sensing module is facing the proper orientation and/or against the check, gingiva, etc. As used herein the strap may be referred to as an elastically adjustable strap, as it may be elastically deformed by stretching the strap over the one or more teeth (by the action of the subject or caregiver applying the device) such that the clastic strap elastically wraps around the tooth and thus holds the pressure sensing module against the tooth.

In some examples the strap may be configured to fit between the teeth in some regions and may include one or more regions around the circumference of the strap. The strap may have different regions with different properties. This is illustrated in FIG. 22. At least one of these regions may be elastic. In the example illustrated in FIG. 22, the device 2200 including a pressure sensing module may also include a strap 2210 that is elastically adjustable and a sensor unit 2204 similar to that shown in FIG. 21. The strap 2210 includes first regions 2243, 2243′ that are relatively thin and second regions 2241, 2241′ that are relatively thick. In some examples, the first region(s) 2243, 2243′ may be configured to fit between the teeth (e.g., interproximally or interdentally), and the second regions 2241, 2241 may be configured to engage the buccal or lingual side of a tooth. In these examples, the first regions are made thinner so as to facilitate placement in the tight interproximal/interdental space. In some examples the thickness of the strap in at least these first regions 2243, 2243′ is 0.5 mm or less (e.g., 0.4 mm or less, 0.3 mm or less, 0.2 mm or less, 0.1 mm or less, etc.), for example 0.2 mm or less. In some examples, the first regions may be relatively less elastic (e.g., more rigid) than the second regions 2241, 2241′, 2241″. Thus, in use, the strap may be elastically expanded from the more elastic first regions while the second regions that are configured to fit between adjacent teeth are strong and relatively less elastic. This may be advantageous in that stronger, more rigid materials may be used for the first regions, which may undergo more frictional stress or wear from adjacent teeth in the interproximal/interdental space. The different regions may be formed of different materials, including different polymeric materials.

FIG. 23 illustrates an example of a device such as the one shown in FIG. 21 attached to a physical model of a subject's tooth. In this example the pressure sensing module 2204 of the device is held against the buccal side of the teeth in the upper jaw, in a mid-posterior region so that the pressure sensing module 2204 may be held against the cheek when worn.

As mentioned above, the pressure sensor modules described herein are not limited to orthodontic or dental applications, but may be used in a variety of different contexts and for monitoring and/or treating different indications where pressure determinations are advantageous. In addition to sleep monitoring/sleep tracking, monitoring and/or tracking athletic activity, and/or clinical patent monitoring, other examples may include medical devices. For example, the pressure sensor modules described herein may be included as part of a medical device for insertion and/or implantation into the body. For example, a pressure sensor module as described herein may be integrated into a stent such as a vascular stent for inserting into the body to provide blood pressure monitoring and/or to determine the effectiveness of the stent (e.g., by using measured pressure changes to determine whether there is stent endothelialization or restenosis, by using measured pressure to determine whether the stent is effectively holding open a blood vessel or collapsing). In some examples the pressure sensor modules described herein may be included as part of a guidewire, catheter and/or cannula. For example these apparatuses, including the pressure sensor module, may be used to determine pressure in the patient's vasculature (e.g., blood vessels, such as but not limited to coronary vessels, peripheral vessels, etc.). These apparatuses, including the pressure sensor module, may be used to determine contact with a blood vessel wall or other tissue during diagnostic procedures (e.g., angiography, colonoscopy) or interventional procedures (e.g., neurostimulation, ablation, heart valve repair, heart valve replacement, endoscopic surgery, robotic surgery, catheter-based ventricular assist devices), or the like. For example, these methods and apparatuses may be used as part of an apparatus such as a catheter balloons, to determine contact with tissue or blood vessel walls (e.g., for ablation of nerves around a blood vessel such as a renal artery, for cardiac ablation to treat arrhythmias). As another example, the pressure sensor module may be placed at or near the distal end of guidewires to determine contact (e.g., against a blood vessel wall or tissue surface) for helping navigate the guidewire and an associated catheter/medical device within the body. In this example, an operator may receive a signal from the pressure sensor module apparatus indicating a sudden increase in pressure (from contact with a wall) and may use this information to help locate the guidewire and turn the guidewire accordingly. Other examples may include, but are not limited to heart implants (e.g., implanted valves, implantable pacemakers, heart pumps, etc.), heart pressure sensors, pulmonary artery pressure monitors, etc.

FIGS. 24A-24D illustrate another example of a dental appliance 2400 including a sealed pressure sensor module, similar to that shown in FIG. 3. In FIG. 24A, the dental appliance 2400 is configured as dental aligner or retainer, including a plurality of tooth-receiving cavities 2401 that is configured to be worn over the patient's teeth. The internal components of the pressure sensor module 2451 are shaped and/or arranged such that they conform generally to the contours of the lingual surfaces of a subject's teeth (e.g., generally U-shaped), as shown schematically in FIGS. 24B-24D. The pressure sensor module may be sealed within a cavity that may be formed integrally with the body of the dental appliance 2400 (e.g., as a single unitary structure), or a sealed pressure sensor module may be formed separately and attached to the body of the dental appliance. In general, the sealed pressure sensor module may be coupled to the dental appliance in any appropriate region. In FIG. 3, the sealed pressure sensor module was included as part of the palatal region. In FIG. 24A, the sealed pressure sensor module is coupled to a lingual side of the dental appliance. Alternatively, the sealed pressure sensor module may be coupled to the buccal side (or to both the buccal and lingual sides).

The scaled pressure sensor module may include a substrate, such as a printed circuit board (PCB) material having an upper surface 2431 and a lower surface 2431′ to which components such as, but not limited to, a pressure sensor 2408, power source (e.g., battery 2411), control circuitry 2405 (e.g., processor, transmitter/receiver, etc.), other sensors (e.g., temperature sensor 2428, force sensor, capacitance sensor, optical sensor), etc. are coupled. At least a portion of the substrate may be configured to be flexible and may have a pattern that permits movement (e.g., flexing, stretching, compression, etc.) of the module. For example, the substrate may be configured to have a serpentine, zig-zag, or curved pattern that may be formed of the flexile material and may allow a range of compression, expansion, and/or bending without disrupting the function and/or connectivity of the attached components. In the example of FIGS. 24A-24D, an intervening portion of the substrate between the two lateral regions 2429A and 2429B of the generally ‘U’ shaped module. The PCB may include traces electrically connecting the components. Both sides of the substrate may be used, as shown in FIGS. 24B (showing the top view of the module) and 24C (showing a bottom view of the module). The use of a flexible substrate may allow the same general pressure sensing module form factor to be used in appliances having a variety of different arch shapes and sizes.

In this example the pressure sensor 2408 is on the bottom surface of the substrate and is held within a sealed chamber 2403. The pressure sensor within the sealed cavity may face the palatal region for sealed pressure sensing. The sealed chamber may be formed within the dental appliance, as described above.

The dental appliance including the sealed pressure sensor module may include any number of additional sensors, including, but not limited to: a pressure sensor, a photoplethysmography (PPG) sensor, accelerometer, etc. For example, a PPG sensor may include one or more optical sensors for detecting blood SpO2. The PPG sensor may be configured to measure reflective PPG and may include an optical emitter and receiver that may capture physiological data from the soft tissue (e.g., gingiva). In some examples, the PPG sensor may detect heart rate. In some examples, the dental appliance may include any appropriate accelerometer, such as but not limited to a piezoelectric, piezoresistive, or capacitive accelerometer. In some cases a triaxial accelerometer may be used. In general, these sensors may be part of the sealed pressure sensor module or may be separately coupled with the dental appliance. In variations in which the sealed pressure sensor module includes the additional sensor(s) (e.g., pressure sensor, PPG sensor, etc.) the additional sensor(s) may be included within the sealed chamber or separate from the sealed chamber.

Any of these apparatuses may include a force sensor which may be coupled to the substrate. The force sensor may be used to detect teeth clenching/grinding (e.g., for determining bruxism) and/or other forces acting on the dental appliance. In some examples, the force sensors may be used to measure forces on teeth from biting, e.g., for determining wear on teeth and/or inferring other oral health conditions generally. In FIGS. 24A-24D the force sensor is positioned on the substrate and configured to face the palatal region for detecting bruxism.

The sealed pressure sensor module shown in FIGS. 24A-24D has a generally U-shaped configuration that follows the contour of the lingual side of a subject's dental arch. In examples where the sensor module is on an upper arch dental appliance, the palatal region is thus not utilized (in contrast with the design shown in FIG. 3). This configuration may provide a compact form factor that may reduce interference when swallowing and may have enhanced comfort as compared to other configurations. In some examples, the sensor module may alternatively or additionally be placed on a lower arch dental appliance. Again, disposing the sensor module components along the lower dental arch allows for reduced interference with features such as the tongue. The substrate 2431, 2431′ may be a single piece (e.g., in FIGS. 24A-24D, having two ‘legs’ connected by a serpentine region) or multiple pieces (e.g., separate legs that may be connected by pieces of substrate and/or wire connectors). All or a portion (e.g., the portion including the pressure sensor) may be encapsulated. In some examples the sealed chamber including the pressure sensor may be formed by double layer thermoforming. For example, the thermoformed base layer may cover the palatal area and includes a narrow margin on the teeth area; the top layer may be thermoformed to create the sealed pressure cavity. In some examples the pressure cavity may be formed under relatively high pressure (e.g., higher than atmospheric pressure) and the high pressure may be released from the cavity afterwards.

The one or more sensors, including the pressure sensor, of the module may be arranged on either side of the substrate. The substrate may support the sensors. In FIGS. 24A-24D the force sensor and the pressure sensor are shown on the back sides of the substrate (e.g., FIG. 24B), on opposite legs (e.g., opposite sides) of the module. In this example a temperature sensor 2428 is on the frontside of the substrate (FIG. 24C). In some examples, an accelerometer may also be included and may be used to determine, for example, head/body position and/or orientation as described elsewhere herein. In some examples, as illustrated in FIG. 24C, the temperature sensor 2428 is configured to be worn at an anterior position near the subject's incisors or canine. This positioning of the temperature sensor 2428 at this anterior position in this examples is deliberately done so that airflow through the mouth due to breathing will introduce local temperature fluctuations that may be sensed by the temperature sensor(s). These local temperature fluctuations, while undesirable in other contexts, may be used to infer certain subject activities or conditions of interest.

For example, FIGS. 25A and 25B illustrate operation of dental appliance such as that shown in FIGS. 24A-24D during nasal breathing (FIG. 25A), when the subject is breathing through their nose, with their mouth closed, and during mouth breathing (FIG. 25B). In FIG. 25A the pressure sensed by the encapsulated pressure sensor in the oral cavity when nasal breathing is consistent with that shown in FIGS. 10A-10B. Similarly, FIG. 25B shows data recorded from the oral cavity during mouth breathing, similar to FIG. 11. The pressure signal is shown both unfiltered 2506 and filtered 2506′ (e.g., filtered using a band pass filter). Nasal breathing may be distinguished from mouth breathing based on the general shapes of the traces. For example, the nasal breathing measurements, shown in the traces in FIG. 25A, illustrate plateaus and flat valleys (e.g., as pressure levels are generally maintained within each phase of inhalation or exhalation when the mouth is closed). By contrast, the mouth breathing of FIG. 25B show sharper peaks and valleys (e.g., as pressure levels within the intraoral cavity are more constantly in flux when the mouth is open, and air is breathed in or out). The pressure change during mouth breathing may result from both pressure and temperature change, as the temperature within the mouth may vary with airflow and the external temperature of the air being inhaled.

In some examples, a second temperature sensor may be positioned along a posterior region of the dental appliance in cases where local temperature fluctuations are not necessary or desirable (e.g., to measure intraoral temperature).

In addition to measuring respiration, as well as determining if the respiration is nasal or oral (mouth) breathing, these methods and apparatuses may also identify oral behaviors while sleeping or awake, such as, but not limited to, clenching of the jaws and grinding of the jaws. FIG. 26 shows an example of the detection of these behaviors using an apparatus similar to that shown in FIG. 24A. In FIG. 26, the subject wearing the device first clenched their jaws three times 2608, and then ground their teeth together three times 2610. In FIG. 26, data from both pressure from the encapsulated pressure sensor and force, form the associated force sensor are shown. The data from the force sensor confirms the pressure data, showing detection of both tooth clenching and grinding. Such data can be used to determine the likelihood of conditions such as bruxism.

As discussed above, any of the apparatuses described herein may include one or more temperature sensors, e.g., integrated into the apparatus. FIG. 27 illustrates the sensing of temperature using an integrated temperature sensor within the dental appliance, as shown in FIGS. 24A-24D. In FIG. 27, the sensed temperature is approximately body temperature (e.g., approximately 36 degrees C.) on the left, when the subject's mouth is closed. After 5:40 (hour 5, 40 minutes), the subject's mouth is opened, and the change, shown as higher frequency changes in temperature reflects respiration through the mouth.

### Identification and Characterization of Apnea

Any of these apparatuses may be used to identify and/or characterize a subject's sleep apnea. FIG. 28A schematically illustrates one example of a method of detecting and/or characterizing apnea in a subject wearing a dental appliance including a sealed pressure sensor module. The method of detecting and/or characterizing apnea may be performed in real time or near-real time in a subject wearing the dental appliance, e.g., during sleep, or it may be detected later. The detection may be performed locally, e.g., in the processor(s) of the dental appliance, and/or remotely in one or more external computing devices (e.g., a smart phone, a computer) in communication with the dental appliance). In FIG. 28A, the method may include determining a single characteristic pulse corresponding to an inhalation and/or exhalation for a first epoch 2801. The single characteristic pulse may be derived from a plurality of respirations (inhalation/exhalations) over an epoch (e.g., 10 seconds, 15 seconds, 20 seconds, 25 seconds, 30 seconds, 45 seconds, 1 minute, 1.5 minutes, 2 minutes, etc.). Thus, the single pulse may a representative pulse selected from or constructed from a set of pulses recorded over the epoch. Alternatively, in some cases the single pulse may be an actual pressure signal recorded. For example, the method may include analyzing and/or characterizing each pulse recorded.

As used herein, a pulse refers to the pressure signals recorded during a period of inspiration and expiration in the subject wearing the apparatus. Both the inspiration and expiration portions of the cycle may be used in the method of detecting and/or characterizing apnea, or in some cases just the inhalation portion or just the exhalation portions may be used. In general the methods described herein may identify a single pulse of a respiration signal from the recorded pressure data. In some cases the pulse may be identified by computing a typical pulse from all or a subset of the pulses recorded during the respiratory cycle. The pressure pulse may be standardized, and may be modified to reduce noise that may otherwise impact the comparison between other pulses.

Any sensed signal that corresponds to respiration may be used by the methods and apparatuses described herein. For example, the signal may be a pressure signal (e.g., from the mouth and/or nose), a temperature signal (e.g., when breathing through the mouth), a force signal, etc. The sensed signal may generally be referred to as a respiratory signal. For example, FIG. 28B illustrates one example of a method of sensing a characteristic pulse from a signal including a plurality of generic pulses corresponding to respiration 2851. In some examples, a single, characteristic, pulse 2858 may be determined as a representative pulse from one or more pulses detected over a period of time (epoch). Alternatively, a characteristic single pulse may be determined by combining (e.g., averaging) a plurality of the pulses detected over the epoch, etc. In FIG. 28B, the characteristic single pulse 2858 is determined by first sensing individual pulses within an epoch 2852, such as by identifying peaks and troughs (marked in the example respiration signal 2851). Individual pulses may extend from trough-to-trough and peak-to-peak and may be identified within the signal using the trough and/or peaks. The identified individual pulses may then be normalized, e.g., temporally and/or in amplitude 2854, and a characteristic single pulse may be generated by, e.g., averaging the normalized pulses from the signal 2856. Signal processing may be applied to reduce the noise and/or amplify the signal. For example, the individual pulses and/or the average pulse may be filtered, smoothed, etc. Morphological features may then be extracted from the resulting characteristic pulse 2858 for an epoch 2860, and the dimensionality of these features may be reduced 2862.

FIGS. 29A-29H graphically illustrate the identification (e.g., construction) of a single pulse of pressure from a plurality of pressure signals detected over a window of time (e.g., 30 seconds). FIG. 29A shows an example of a pressure signal recorded over an epoch (e.g., a suitable time window over which pulses are analyzed, e.g., 30 seconds, 1 minute, 5 minutes) from a patient wearing a dental appliance including a sealed pressure sensor module as described herein. The signal shows amplitudes for the pressure measured, and includes peaks and troughs sensed by the scaled pressure sensor module that result from phases of the respiratory cycle (e.g., inhalation and exhalation). In FIG. 29B, individual raw pulses (seven pulses are shown) from the window of time shown in FIG. 29A are shown approximately overlaid. These individual pulses may then be cleaned up and aligned, so that a single representative pulse may be identified. For example, outliers may be removed, the pulses may be normalized (e.g., amplitude and/or temporal normalization), and further signal process (e.g., filtering, smoothing) may be employed. Once normalized and processed, the resulting subset of pulses may be averaged to generate a single characteristic pulse, shown in FIG. 29C.

The single characteristic pulse (e.g., the pulse in FIG. 29C) corresponds to a normalized, noise-reduced signal characteristic of the epoch (e.g., 30 seconds) over which it was estimated. FIG. 30 illustrates an example method where single characteristic pulses are generated separately for each of multiple phases of breathing (e.g., inspiration and expiration) and then concatenating them into a single combined pulse that includes information about the multiple phases (e.g., inspiration and expiration). For example, FIG. 30 shows a pressure signal 3005 of the window of time (e.g., 30 seconds) including a plurality of expiration and inhalation regions. The troughs may correspond to inspiratory flow features 3007, and the peaks may correspond to expiratory flow features 3009. As described above, these features may be used to determine an inspiratory single pulse and an expiratory single pulse for the epoch. In some examples, the inspiratory single pulse and the expiratory single pulse may be concatenated by overlaying them (e.g., as visualized in the graph 3010), resulting in a single combined pulse that includes information about both inspiration and expiration. Alternatively, the inspiratory single pulse and the expiratory single pulse may be combined in any other suitable manner to yield a single combined pulse. In some examples, a single characteristic pulse is determined without having to separately determine single pulses for different breathing phases and then combining them. That is, a single characteristic pulse for an epoch may be directly determined based on, e.g., the raw pressure signals from the epoch.

Returning to the method outlined in FIG. 28A, features of a single characteristic pulse (e.g., the single combined pulse 3111) may then be extracted 2803. Extracted features may generally correspond to morphological features of the single characteristic pulse and may be used for comparison in order to determine a probability that the pulse corresponds to apnea. Any appropriate feature extraction technique may be used, including, e.g., extracting morphological characteristics using convolution (e.g., using a convolutional encoder as shown in FIG. 31). In this example, the selected single pulse 3111 may be convolved multiple times using an encoder 3113 to extract a reduced number of characteristics to create a reduced order representation (e.g., the latent feature representation 3113 in FIG. 31, which includes a subset of features extracted following the multiple convolution steps FIG. 31). These extracted characteristics may optionally then be standardized, and then analyzed, e.g., using principal component analysis (PCA). In some examples, a subset of the features of the PCA analysis, e.g., two of the PCA features, may be extracted as characteristic features in order to categorize the single pulse so as to reduce dimensionality (and thus saving computing time and resources). In FIG. 31, the two terms of the PCA analysis may be tracked over time for each epoch allowing comparison of individual single pulses over time, e.g., during sleep. For completeness, FIG. 31 also shows that the extracted morphological features from the single pulse (e.g., the features of the latent feature representation) may be decoded, by deconvolution 3115 back to the original single characteristic pulse 3111′.

As shown in the method outlined in FIG. 28A, the reduced-dimension extracted features (e.g., from the PCA analysis or otherwise) may then be used to characterize the single characteristic pulse 2805, and these characteristics may be used to determine a probability of apnea 2807. Although the disclosure focuses on apnea, it contemplates that the methods disclosed herein (e.g., mapping, clustering, cluster labeling, and classification) can be applied to any other suitable condition (e.g., breathing disorders).

FIG. 32A illustrates an example of mapping pulses on a feature space 3200 (e.g., in the illustrated example, reduced to two dimensions Feature 1 and Feature 2) and clustering the pulses based on their mapped position in the feature space during a training phase using a supervised learning method. Referencing the feature space 3200, each data point (represented as triangles, squares, and small circles) corresponds to a single pulse that may have been sampled from one or more subjects using the disclosed pressure sensor. The locations of each data point reflect the feature values (the values of Feature 1 and Feature 2) of the corresponding pulse. In this training process, each of the pulses have been characterized as being associated with normal, apnea, or hypopnea events based on ground truth data (e.g., data known to indicate normal, apnea, or hypopnea) that may have been determined using, for example, reference sleep monitoring devices. For example, in the example shown in FIG. 32A, a reference sleep monitoring device has been used to determine that the pulse 3232 likely corresponds to a hypopnea event, the pulse 3234 likely corresponds to normal breathing, and the pulse 3236 likely corresponds to an apnea event. All of the sampled pulses may be clustered (e.g., referencing FIG. 32A, Clusters 1, 2, and 3) based on their similarities in features (as represented by relative proximity in an n-dimensional space where n corresponds to the number of features, e.g., the 2-dimensional space of the feature space 3200). In some cases, the reference data is used to determine the cluster boundaries (e.g., size, shape of the cluster). For example, the boundaries of Cluster 3 may be determined in part by the high concentration of the labeling of hypopnea and apnea events.

FIG. 32B illustrates an example process of training a model to label identified clusters (e.g., labeling them with apnea probability values) based on the number of apnea and hypopnea events within each cluster. Once pulses for a particular epoch have been clustered (e.g., as explained with respect to FIG. 32A), one or more of the clusters may be labeled based on a likely characteristic. For example, the clusters may be labeled based on a likelihood that a future hypothetical pulse falling within the cluster will result in apnea (or hypopnea). In some examples a cluster may be labeled low, medium, high, or very high to indicate the probability that a pulse having features that map it within the cluster corresponds to an apnea event. The probability may be expressed numerically (e.g., between 0 to 1, as a percentage, as a score, etc.). In one method, as illustrated in FIG. 32B, the number of apnea and hypopnea events are counted, and then an associated probability of apnea is determined by dividing that number by the total time represented by the pulses in the cluster (e.g., if the cluster consists of 100 pulses that are each 5 seconds, the total time represented by the pulses in the cluster is 500 seconds). For example, if a cluster has 10 apnea and hypopnea events and the total time of the cluster is 500 seconds, a value of 0.02 may be calculated. This value may then be correlated to a probability of apnea for the cluster. As explained further below with respect to FIG. 33, after the clusters have been defined in the feature space following the training, pulses received from a future subject wearing a sensor module (e.g., the disclosed pressure sensor, a force sensor, or another sensor that correlates to respiration) can be mapped into the feature space. If a particular pulse falls into a cluster (or if it is within a threshold distance from its closest cluster), the probability associated with the cluster may be associated with the particular pulse. For example, if the pulse has features that fall within Cluster 3, it may be determined that there is a high probability that the pulse is associated with apnea. FIG. 32C illustrates one example showing classification of clusters with apnea probability, using a graph similar to that shown in FIG. 32A.

FIG. 33 schematically illustrates an example of a technique for predicting the probability for apnea in a sleeping subject using clustering. As explained previously with respect to FIG. 32B, the probability for a cluster may be based on a number of apnea/hypopnea events per total time of a cluster. In the example of FIG. 33, a cluster with less than 5 events per hour is determined to have a low probability, a cluster with 5 or more events but less than 15 events is determined to have a medium probability, a cluster with 15 or more events but less than 30 events is determined to have a high probability, and a cluster with more than 30 events is determined to have a very high probability. As explained previously, a model may be created by mapping training data that has been annotated by a reference device (e.g., a sleep apnea monitoring device) based on their features in a feature space, clustering the training data within the feature space, and then labeling the clusters with an apnea probability (e.g., low, medium, high, very high). Once this model has been created, a pulse from a subject (“Subject Data”) acquired by an intraoral device (e.g., using a pressure sensor, force sensor, etc.) may be mapped onto the feature space and classified based on a corresponding cluster. In this example, the pulse falls into a cluster that is associated with a medium probability. The pulse may thus be classified as having a medium probability of corresponding to an apnea event. In some examples, pulse that does not fall within a cluster, but is nonetheless within a threshold distance of a cluster (e.g., its closest cluster) will be classified as having the probability associated with that cluster. Although the clustering models shown in FIGS. 32A-32C and 33 are two dimensional (e.g., using the first two components of a PCA), more than two dimensions may be used (e.g., three dimensions, four dimensions, etc.), using additional components of the PCA and/or alternative characteristics. In some examples, the PCA may not be used to reduce the dimensionality, but the full set of characteristics of the latent representation may be used.

Any of these methods may also or alternatively be performed using a trained machine learning agent (e.g., trained neural network). For example, a machine learning agent may be trained on the training data and used to recognize and label each pulse.

The method described above may be used to estimate the probability of apnea in a continuous manner, e.g., scoring each epoch (e.g., 30 seconds, 1 minute, 5 minutes) during sleep, and/or may provide an overall score of apnea. The score may alternatively be referred to as a score of the severity of apnea, as it may indicate the number and/or duration and/or intensity of apnea episodes during sleep.

FIGS. 34A-34D illustrate different examples presenting the results of the methods described herein, showing exemplary outputs that may be included as part of the method. FIG. 34A shows a chronologic illustration of apnea probability over time (e.g., per epoch), illustrating the probability of the apnea for each pulse as a different color/shade. FIGS. 34B and 34C are bar and pie graphs, respectively, showing the duration of each apnea probability for a particular subject over a particular sleeping period. FIG. 34D shows apnea probability correlated to body position (e.g., which may be measured by an accelerometer on the dental appliance) during an 8 hour sleep period. In this example, the positions include supine, lateral, and prone positions, and the distribution of apnea probabilities for each position are illustrated. The examples of methods of visualizing/displaying the results shown in FIGS. 34A-34D are representative only, and other visualizations may be used, including textural and/or graphical.

In general, the apparatuses described herein may be configured to perform any of these methods. For example, the apparatuses described herein may include a sleep apnea detection and/or classification module configured to perform any of the steps described above, including identifying the representative pulse (e.g., over an epoch that may be predetermined or set by the user, e.g., clinician), and may include feature extraction, and characterization and estimation of sleep probability. Although the discussion above focuses on apnea, the disclosure contemplates that pressure data may be used to determine probability of any suitable condition (e.g., a respiratory condition) using similar methods (e.g., extracting features of pulses, and clustering the pulses as described above).

### Multi-Sensor Apparatuses and Methods

As mentioned, the apparatuses and techniques described above may be used to detect one or more physiological states or conditions using data from one or more sensor on the dental appliance. These sensors may include, but are not limited to: the sealed pressure sensor module, one or more accelerometer, one or more temperature sensors (thermistors, resistance temperature sensor, thermocouple, infrared sensor, etc.), one or more photoplethysmography sensors (PPG), etc. As described above with reference to FIGS. 1A, 3, 4A-4C and 24A-24D, the dental appliances described herein may include any of these sensors as well as the control circuitry for operating these sensors. The dental appliances may also include one or more modules (e.g., software, hardware and/or firmware) to record, transmit and/or analyze the data received from these sensors to monitor or detect one or more physiological states or conditions.

The methods to analyze data derived from these sensors, and in particular, to analyze combinations of data derived from these sensors, may be performed locally (e.g., on the dental appliance) or remotely (e.g., on a processor remote to the dental appliance that receives data from the dental appliance, such as a wearable processor (e.g., smartwatch, monitor, etc.), tablet, laptop computer, desktop computer, cloud-based processor, etc. Processing of the data may be divided between a local and remote processor.

Examples of the one or more physiological states or conditions that these apparatuses may detect and/or monitor may include, but are not limited to: respiration rate, mouth breathing, sleep stage, sleep apnea, sleep position, core body temperature, and bruxism. Detection of the physiological states/conditions as described herein may provide insight and help determine the underlying cause of a condition (e.g., sleep monitoring, bruxism, mouth breathing, etc.) and in some cases may be used treat the underlying cause.

FIGS. 35A-35B show an example of the detection of respiration rate using an apparatus as described herein. Respiration rate may provide the number of breaths per unit of time (e.g., per minute), and is a vital sign that may indicate a person's respiratory health, as well as fitness level. In FIG. 35A, the sealed pressure sensor (e.g., barometer) on the dental appliance may record breathing cycles (e.g., pulses) 3501 as described above and may detect individual cycles, including sensing maxima (and/or maxima greater than a threshold), and minima (including minima less than the same, or a different, threshold). The sensor data may be processed using one or more signal processing techniques (e.g., filtering, such as Kalman filtering 3503), and a respiratory rate may be determined. This may be performed in real time. FIG. 35B is a graph (spectrogram) showing tracking of pressure over time using a dental appliance as described herein, indicating respiration rate. The respiratory rate estimation tracks the spectral peaks of the spectrogram in this example.

FIG. 36A schematically illustrate an example of detection and/or monitoring of mouth breathing. Mouth breathing includes inhaling and exhaling through the mouth, rather than the exclusively through the nose. Nasal breathing may be considered an optimal mode of breathing. When breathing through the mouth, the correlation between the barometer and temperature sensor increases, and this correlation may provide an estimate of mouth/nose breathing. In FIG. 36A the method includes processing (e.g., pre-processing) 3603 both pressure, e.g., from the scaled pressure sensor of the dental appliance, and temperature from the dental appliance. This may include filtering of the breathing components. The pressure and temperature signals may be aligned (e.g., in time) 3605, and a correlation coefficient may be calculated 3607 from the aligned signals. The correlation coefficient may then be used with a classification model 3609 to characterize the subject's breathing as mouth and/or nose breathing. FIG. 36B shows an example of aligned temperature and pressure traces (top) and the maximum correlation coefficient (bottom), showing regions of nasal inhalation/exhalation, oral (mouth) inhalation/exhalation, nasal inhalation/oral exhalation, and oral inhalation/nasal exhalation.

These methods and apparatuses may also be used to detect and/or monitor, including characterizing, sleep apnea, as described above. FIG. 37A schematically illustrates another example of detecting sleep apnea from the pressure signal using a sealed pressure sensor as described herein. In FIG. 37A, the method may generally include preprocessing of the pressure signal, e.g., to determine the power spectral density 3701, detect up/down intercepts 3703, detect peak and troughs 3705, and remove outliers 3707. Features may then be extracted, e.g., including but not limited to a time-domain respiratory rate variability 3711, performing spectral analysis 3713, performing a non-linear analysis 3715, and/or determining amplitude variability 3717. FIG. 37B shows an example of a respiratory signal and the resulting spectral analysis.

As mentioned above, the methods described herein may include the use of a trained model (e.g., a trained machine learning agent). FIG. 37C schematically illustrates an example of training a model and applying the trained model to detect and/or monitor apnea (e.g., detecting periods of normal respiration and/or apnea AHI).

These methods and apparatuses may also be used to detect and/or monitor sleep stages of a subject wearing the dental appliance. Sleep stages typically occur in a cyclic pattern throughout the night and include periods of Awake, REM, and NREM (N1, N2, N3) sleep. Monitoring sleep stages may help assess the quality of sleep. FIG. 38A schematically illustrates one example of a method for detecting sleep stages using a multi-sensor apparatus. In this example, the method includes using movement data from an accelerometer and extracting features 3801 such as activity level and body inclination angle, using pressure data from the sealed pressure sensor and extracting feature such as respiration rate 3803 and respiration rate variability 3805. Temperature data from one or more temperature sensors on the apparatus may be processed 3807 to determine core body temperature 3809. All of these physiological indicators may then be used with a trained classification model 3811 to determine the sleep stage 3811 of the subject wearing the dental appliance.

Sleep position is another physiological state that may be detected using these apparatuses and methods. For example, FIGS. 39A-39B illustrate detection of posture or bodily orientation during sleep, e.g., supine, left/right lateral, prone, using an accelerometer on the dental appliance. FIG. 39A shows the extraction of features indicating rotation angle 3901 and/or inclination angel 3903, and the use of a trained classification model 3905 to determine body position (e.g., supine, left lateral, right lateral, prone) from the extracted features. FIG. 39B illustrates the corresponding movements that may be determined, including head inclination 3911, head rotation 3913, and head positioning within a coordinate system 3915. Sleep position can influence the case of breathing and airway function during sleep. For example, sleeping in a left lateral position can help reduce symptoms of acid reflux.

As mentioned, these apparatuses and methods may estimate core body temperature using intraoral temperature sensor on the dental appliance. FIG. 40 schematically illustrates the detection of core body temperature, including receiving an intraoral temperature, e.g., from a temperature sensor on the intraoral appliance. The temperature data may then be processed, e.g., by removing outliers, smoothing/filtering, etc. 4001. The temperature data may be combined with the detection of mouth breathing 4005; as mentioned above, core body temperature from an intraoral sensor may be accurately detected when the mouth is closed (e.g.,. when nose breathing); in some cases temperature data recorded during mouth breathing may be removed. The temperature data may be used along with respiratory rate 4007 estimates to determine core body temperature using a core body temperature estimation model 4003. In some examples the core body temperature estimation model may be trained using reference data.

As described in reference to FIGS. 26, these apparatuses and methods may detect bruxism (e.g., teeth grinding or clenching). Bruxism may damage a subject's teeth and may disrupt the person's sleep patterns. In some cases, Bruxism may be sensed by the pressure signal (e.g., from a sealed pressure sensor, as described) and/or a force sensor. FIG. 41 shows another example showing detection of bruxism using a force sensor on the dental appliance; the boxed region 4101 shows the force signal during bruxism.

The apparatuses and methods described herein may also or alternatively be configured for the detection of the subject's sleep stage; as used herein sleep stage may include the awake state, sleep onset, REM sleep and non-REM sleep. Other sleep stages may be indicated. Sleep stage may be determined, for example, from one or more sensor inputs, including respiratory rate (e.g., from sealed pressure sensor module), heart rate/heart rate variability (e.g., from a PPG sensor), blood pressure and/or SpO2 (e.g., from a PPG sensor), body position and/or movement (e.g., from an accelerometer), and/or core body temperature (e.g., from a temperature sensor). For example, the apparatuses described herein (e.g., dental appliances) may include a sleep stage classification module that is configured to receive input from one or more of these sensors and may include sleep stage classification logic that may estimate sleep stage based on the sensor date. The sleep stage classification module may output a time or percentage in one or more of these stages.

Similarly, these methods and apparatuses described herein may be configured to determine sleep position for the subject based on data from one or more of the sensors, including (but not limited to) the sealed pressure sensor module. Sleep position may be classified by the system into supine, lateral and/or prone positions, and may be inferred from accelerometer data, which may be combined with additional sensor data, and/or with output from the sleep classification module. For example, body position data (e.g., from an accelerometer in the dental apparatus) may be interpreted to provide body position data when the subject is sleeping, e.g., as indicated by the sleep classification module. The sleep position classification module may output a time and/or percentage of time sleeping in one or more of these positions.

In some cases the methods and apparatuses described herein may also be configured to determine when the subject is breathing through the mouth and/or nose. For example, as illustrated in FIGS. 10A-10B and 11 and 25A-25B, the pressure data from the sealed pressure sensor may distinguish between mouth (oral) and nasal respiration. A mouth breathing detection module may determine nasal or oral (e.g., nasal/oral) breathing. The mouth breathing detection module may also indicate a time and/or percentage of time breathing through mouth vs. nose.

FIG. 42 illustrates examples of the sensors that may be included in any of these dental apparatuses (e.g., sealed pressure sensor module, PPG sensor, temperature sensor and accelerometer), types of biological parameters that may be derived from these sensors (e.g., blood pressure, heart rate, heart rate variability, respiratory rate, etc.), and sleep-related phenomena that may be derived from these biological parameters (e.g.,, sleep stage classification, sleep quality classification, sleep apnea/hypopnea/desaturation, and AHI index/oxygen desaturation index). Thus, described herein are sleep monitoring systems that can be incorporated into any of the dental appliances described herein to continuously monitor changes of any of these physiological parameters over time.

For example, a sleep monitoring apparatus can include a dental appliance that may be configured to include a sealed pressure sensing modulate, a PPG sensor, an accelerometer, and a temperature sensor, and can extract features for sleep stage classification and sleep apnea/hypopnea detection, or other parameters. For example, sleep quality can be quantified using several parameters, including sleep latency, sleep waking, wakefulness, sleep efficiency. Apnea-hypopnea index (AHI) and oxygen desaturation index (ODI) can be calculated using the prediction results of sleep apnea/hypopnea detection and desaturation event detection algorithms. These apparatuses may provide not only multiple vital signs such as blood pressure, heart rate, heart rate variability, SpO2, respiratory rate, and core body temperature, but also advanced features like sleep stages and sleep apnea with compact and portable devices.

In general, these apparatuses may fit inside the subject's mouth without interfering with sleep or causing discomfort, and may provide accurate measurements for sleep monitoring. Compared with vital signal monitoring at other body locations (e.g., wrist, chest, ear), intraoral monitoring may provide a more convenient and comfortable way for long term usage, as well as more accurate data that immune to motion artifacts.

For example, blood pressure related features can be extracted from a PPG sensor receiving a PPG signal. The PPG signal may include components useful for blood pressure estimation may include the systolic peak, diastolic peak, dicrotic notch, and the time intervals between systolic and diastolic peaks. In some examples, blood pressure may be derived from the PPG, and may be useful for sleep monitoring. For example, the relative level of blood pressure may be indicated if the subject is awake of asleep. An awake subject may generally have a higher blood pressure than an asleep subject. The sleep stage, e.g., Non-Rapid Eye Movement Sleep (NREM) and REM may be related to blood pressure measurements as well. For example, decreases during NREM sleep may indicate a reduced sympathetic nervous systema activity and may be reflected in a lowest blood pressure during slow-wave sleep. In contrast, Rapid Eye Movement Sleep (REM) may be identified by a higher blood pressure, as blood pressure may rise to levels comparable to wakefulness or even higher during REM. Normally, blood pressure may dip by at least 10% during sleep. Sleep apnea (e.g., obstructive sleep apnea, OSA) is associated with high blood pressure.

Heart rate can be estimated using the spectral peak of the PPG signals. Heart rate variability, HRV, refers to the variation in the time intervals between consecutive heartbeats and is an indicator of autonomic nervous system activity. Heart rate is higher in the awake subject compared to other stages. Heart rate during Non-Rapid Eye Movement Sleep (NREM) gradually decreases as an individual transition from wakefulness to NREM sleep. During slow-wave sleep, heart rate typically reaches its lowest levels. During Rapid Eye Movement Sleep (REM) the HRV may be low and variable. HR is higher and HRV is lower during sleep apnea.

As mentioned, SpO2 can be estimated using the IR and red PPG sensors. It is normal for oxygen levels to slightly decrease during sleep compared to wakefulness. However, significant drops in SpO2 levels can indicate underlying sleep disorders such as sleep apnea and hypopnea. During REM sleep, the oxygen saturation is generally lower than during NREM sleep. Respiratory-induced variations (frequency, intensity, and amplitude) in PPG signal can be used to estimate respiratory rate. Also, changes in pressure caused by breathing can also or alternatively be used. An intraoral dental apparatus can be affected by factors such as snoring or speaking, which can cause changes in pressure levels. By combining PPG and barometer measurements, the methods and apparatuses described herein may estimate respiratory rate very robustly.

Respiratory rate variability in OSA is typically higher than normal. Respiratory rate may be lower during OSA and can be increased as the body tries to compensate for the obstruction.

As mentioned, any of these apparatuses may be configured to determine body position. For example, any of these dental apparatuses may include an accelerometer that can be used to classify sleep position such as supine, left, and right. These sleep positions can be used as a factor to manage or alleviate symptoms of sleep apnea. For example, these methods and apparatuses may receive a preferred sleep position from the subject. Sleeping on the side, particularly the left side, can help reduce the occurrence of obstructive sleep apnea (OSA) events. This is because the tongue and soft tissues are less likely to relax and obstruct the airway in this position. The dental appliances described herein may measure the head orientation and inclination accurately to help with identifying a right treatment method or tool. In some variations these apparatuses and methods may provide the subject with output indicating the body position and/or apnea occurrence (see, e.g., FIG. 34). In some cases the apparatus and/or method may include suggesting ways for the subject to reduce apnea and/or snoring. For example, the apparatus or method may suggest a pillow height that can be adjusted to prevent or reduce the OSA events even in the same sleeping position.

Accelerometer output may provide measurements of acceleration along three axes (X, Y, and Z). A movement level can be estimated using the Euclidean distance of three axes. As sleep onset approaches, movement may gradually decrease. During REM sleep, the body may experience temporary muscle paralysis known as REM atonia. This inhibits significant bodily movement, except for minor twitches or eye movements. The methods and apparatuses described herein may detect body movement and therefore derives sleep stage. In addition, these methods and apparatuses may identify disorders from this information, such as, but not limited to, Periodic limb movement disorder (PLMD) or restless legs syndrome (RLS), sleepwalking, sleep talking, and sleep bruxism. OSA can lead to brief awakenings, accompanied by movements as the body tries to restore normal breathing.

As mentioned above, these methods and apparatuses may monitor temperature, e.g., core body temperature. For example, an intraoral temperature sensor may be located on a dental appliance worn inside the mouth, near the body's core temperature. This temperature sensing may be less affected by external factors such as ambient temperature or temperature variations in the environment. The mouth has a rich blood supply, and the temperature of the oral cavity is regulated by a continuous flow of blood. Skin temperature is influenced by sweat evaporation. Intraoral temperature measurement may avoid this factor. Core body temperature typically follows a circadian rhythm, which is a 24 h biological cycle that regulates various physiological processes, including sleep (gradually decreases as the evening approaches). Core body temperature may change during the different stages of sleep, e.g., temperature may continue to decrease during NREM sleep, reaching its lowest point in the early morning hours. Individuals with insomnia my experience higher body temperature at night. Thus, the methods and apparatuses described herein may provide a report on the core body temperature during sleep, may track temperature to assist in identifying sleep stage, and may be configured to identify disorders associated with body temperature (including fever, and disruptions of sleep).

As mentioned, the methods and apparatuses described herein may be used to determine and indicate a sleep stage for the subject wearing the apparatus. In some cases PPG features related to blood pressure can be utilized. For example, a PPG stiffness index (e.g., time delay between the systolic peak and diastolic peak), a reflection index (e.g., amplitude ratio between the systolic and diastolic peaks), pulse amplitude, pulse width, area under curve, normalized harmonic area, and inflection point area are associated with the blood pressure and may be determined and used by the methods and apparatuses described herein. The most relevant features may be selected for sleep stage classification. This can be done by correlation analysis, feature importance ranking. Various algorithms can be used for the classification model such as support vector machine (SVM), random forest, convolutional neural network, and recurrent neural network. The output of the classification model can be five stages: Wake, N1, N2, N3, and REM sleep. The model may be evaluated using F1-score, precision, recall, and confusion matrix. The model may be tested using independent test set to assess its performance on unseen data.

Sleep quality can be quantified using several parameters, including sleep latency, sleep waking, wakefulness, sleep efficiency. Sleep Latency may refer to how long it takes one to fall asleep. Sleep Waking may refer to how often one wakes up during the night. Wakefulness may refer to how many minutes one spends awake during the night. Sleep Efficiency may refer to the amount of time one spends actually sleeping while in bed. The methods and apparatuses may use any combination of the movement (accelerometer) and sleep stage classification to estimate one or more of these sleep quality parameters.

The methods and apparatuses described herein may also include a sleep apnea/hypopnea detection model that can utilize similar features as sleep stage classification model. Features related to blood pressure in PPG, heart rate, heart rate variability, respiratory rate, as well as additional features like SpO2 and sleep position can be used in the detection of sleep apnea and hypopnea. As mentioned, the apnea-hypopnea index (AHI) is the combined average number of apneas and hypopneas that occur per hour of sleep. According to the American Academy of Sleep Medicine (AASM) it is categorized into mild (5-15 events/hour), moderate (15-30 events/hour), and severe (>30 events/hour). The prediction results of sleep apnea/hypopnea detection model may be used to calculate the AHI during sleep.

The Oxygen Desaturation Index (ODI) is the average number of desaturation episodes occurring per hour. Desaturation episodes are defined as a decrease in the mean oxygen saturation of >3% (over the last 120 seconds) that lasts for at least 10 seconds. Desaturation events can be detected using PPG-based SpO2.

The methods and apparatuses described herein, including dental appliances having a sealed pressure sensor module, may be integrated with a therapeutic element for treating a breathing and/or sleeping disorder. For example, the apparatuses described herein may be configured to include a mandibular advancement member in order to treat sleep apnea. A mandibular advancement device has been successfully used to treat obstructive sleep apnea by moving the mandibular forward. However, it may be undesirable to wear this type of device for a long period. An active member can be integrated into the mandibular advancement device to displace the mandibular only when needed instead of continuously applying a load. For example the methods and apparatuses for sensing apnea described herein can be used as a feedback mechanism to detect obstructive sleep apnea in a sleeping subject and may activate the mandibular advancement device for treatment in real time, as needed.

The dental appliances described herein may be used in a variety of cases and applications. For example, these methods and apparatuses may be configured to track things like steps, basic movement, resting and/or casual heart rate, etc. Thus, in some examples, these methods and apparatuses may be useful to track movement while wearing the apparatus and report to the subject any of these movement indicators for health and fitness analysis.

In some examples real-time biometric measurements may be provided during a specific activity such as working out, running, biking, etc. Stability and accuracy are highly valued in these scenarios, and the dental appliances described herein may provide a convenient and highly accurate measure of activity and biometric information (e.g., pulse/heart rate, blood pressure, body temperature, etc.). In some cases these methods and apparatuses may be used for determining one or more personal health metrics. For example, these methods and apparatuses may provide a metric of personal health indicators such as heart rate, blood pressure, oxygen saturation, etc. These measurements can be used in conjunction with a prevention plan (in healthy populations) or a disease management plan (for those managing a health condition such as hypertension, diabetes, cardiovascular disease, etc.).

The methods and apparatuses described herein may use any of the detection models described above in any combination, which may provide additional information to the patient and/or clinician, including information about potential causes of the condition being managed, including but not limited to apnea, and/or may provide information to treat the condition.

For example, FIG. 43 schematically illustrates methods of inferring or detecting causes or effects of apnea using some of the measurement and models described above. For example, in FIG. 43, a model for classifying sleep stage (e.g., a sleep stage classification model 3622) or a module configured to perform this method, may be combed with a sleep apnea detection model, or a module configured to perform this method, to detect arousals in the patient caused by apnea. As shown in FIG. 43, correlation, e.g., in time, between awake periods determined by the sleep stage classification model/module with a determination of sleep apnea from the sleep apnea detection model/module may detect or predict apnea arousal.

Similarly, as shown in FIG. 36, the output of the sleep apnea detection model/module and the sleep position classification model or a module configured to perform the method may result in the detection of apnea caused by the position of the subject (e.g., the subject being in a supine position while sleeping). Other combinations of models/modules may provide evidence of other inferences. For example, the sleep apnea detection model/module and the sleep position classification model/module may also be combined with the mouth breathing detection model, or a module configured to perform this method to provide an indicator that the subject's apnea is caused by mouth breathing.

Thus, in general, any of the methods (and modules configured to perform them) described herein may be integrated together in a combinations or sub-combination that may provide additional patient-specific information about the effects and possible causes of the subject's apnea.

While various embodiments have been described and/or illustrated herein in the context of fully functional computing systems, one or more of these example embodiments may be distributed as a program product in a variety of forms, regardless of the particular type of computer-readable media used to actually carry out the distribution. The embodiments disclosed herein may also be implemented using software modules that perform certain tasks. These software modules may include script, batch, or other executable files that may be stored on a computer-readable storage medium or in a computing system. In some embodiments, these software modules may configure a computing system to perform one or more of the example embodiments disclosed herein.

As described herein, the computing devices and systems described and/or illustrated herein broadly represent any type or form of computing device or system capable of executing computer-readable instructions, such as those contained within the modules described herein. In their most basic configuration, these computing device(s) may each comprise at least one memory device and at least one physical processor.

The term “memory” or “memory device,” as used herein, generally represents any type or form of volatile or non-volatile storage device or medium capable of storing data and/or computer-readable instructions. In one example, a memory device may store, load, and/or maintain one or more of the modules described herein. Examples of memory devices comprise, without limitation, Random Access Memory (RAM), Read Only Memory (ROM), flash memory, Hard Disk Drives (HDDs), Solid-State Drives (SSDs), optical disk drives, caches, variations or combinations of one or more of the same, or any other suitable storage memory.

In addition, the term “processor” or “physical processor,” as used herein, generally refers to any type or form of hardware-implemented processing unit capable of interpreting and/or executing computer-readable instructions. In one example, a physical processor may access and/or modify one or more modules stored in the above-described memory device. Examples of physical processors comprise, without limitation, microprocessors, microcontrollers, Central Processing Units (CPUs), Field-Programmable Gate Arrays (FPGAs) that implement softcore processors, Application-Specific Integrated Circuits (ASICs), portions of one or more of the same, variations or combinations of one or more of the same, or any other suitable physical processor.

Although illustrated as separate elements, the method steps described and/or illustrated herein may represent portions of a single application. In addition, in some embodiments one or more of these steps may represent or correspond to one or more software applications or programs that, when executed by a computing device, may cause the computing device to perform one or more tasks, such as the method step.

In addition, one or more of the devices described herein may transform data, physical devices, and/or representations of physical devices from one form to another. Additionally or alternatively, one or more of the modules recited herein may transform a processor, volatile memory, non-volatile memory, and/or any other portion of a physical computing device from one form of computing device to another form of computing device by executing on the computing device, storing data on the computing device, and/or otherwise interacting with the computing device.

The term “computer-readable medium,” as used herein, generally refers to any form of device, carrier, or medium capable of storing or carrying computer-readable instructions. Examples of computer-readable media comprise, without limitation, transmission-type media, such as carrier waves, and non-transitory-type media, such as magnetic-storage media (e.g., hard disk drives, tape drives, and floppy disks), optical-storage media (e.g., Compact Disks (CDs), Digital Video Disks (DVDs), and BLU-RAY disks), electronic-storage media (e.g., solid-state drives and flash media), and other distribution systems.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

The processor as described herein can be configured to perform one or more steps of any method disclosed herein. Alternatively or in combination, the processor can be configured to combine one or more steps of one or more methods as disclosed herein.

When a feature or element is herein referred to as being “on” another feature or clement, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under”, or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

In general, any of the apparatuses and methods described herein should be understood to be inclusive, but all or a sub-set of the components and/or steps may alternatively be exclusive and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A dental appliance comprising:
a body forming one or more tooth-receiving cavities configured to be worn on a subject's teeth;
a sealed pressure sensor module coupled to the body, the sealed pressure sensor module including:
an airtight encapsulated cavity, wherein the airtight encapsulated cavity is encapsulated by one or more fluid-resistant materials, wherein at least a portion of the one or more fluid-resistant materials forms a deformable surface;
a pressure sensor encapsulated within the airtight encapsulated cavity and configured to measure pressure within the airtight encapsulated cavity,
wherein the sealed pressure sensor module is held within a subject's mouth when the one or more tooth-receiving cavities are worn on the subject's teeth.

2. The dental appliance of claim 1, further comprising a processor configured to receive pressure data from the pressure sensor and configured to modify, store and/or transmit the pressure data.

3-4. (canceled)

5. The dental appliance of claim 1, further comprising a palatal region extending between a first tooth-receiving cavity and a second tooth-receiving cavity of the one or more tooth-receiving cavities.

6-9. (canceled)

10. The dental appliance of claim 1, wherein the sealed pressure sensor module is positioned on a buccal or lingual side of the body.

11. The dental appliance of claim 1, further comprising one or more additional sensors within the sealed pressure sensor module.

12. The dental appliance of claim 1, further comprising one or more of: a temperature sensor, a photoplethysmography (PPG) sensor, a force sensor, and/or an accelerometer.

13. The dental appliance of claim 1, wherein the airtight encapsulated cavity has a volume of between about 0.1 cm³ and 100 cm³ in an uncompressed configuration.

14. The dental appliance of claim 1, wherein the deformable surface comprises a polymeric material having a durometer of between about 20 and about 90 Shore 00.

15. The dental appliance of claim 1, wherein the deformable surface has a surface area of between about 1 mm² and 1.5 cm².

16-19. (canceled)

20. The dental appliance of claim 1, wherein the sealed pressure sensor module is laminated to a body forming the one or more tooth-receiving cavities.

21-36. (canceled)

37. A method of monitoring intraoral pressure using a dental appliance, the method comprising:
receiving a signal from a pressure sensor sealed within an airtight encapsulated cavity of a pressure sensor module coupled to a dental appliance being worn by a subject, wherein the airtight encapsulated cavity is encapsulated by one or more fluid-resistant materials; and
determining, from the signal, intraoral pressure.

38. The method of claim 37, wherein determining intraoral pressure further comprises determining a respiration pattern.

39. The method of claim 37, wherein determining intraoral pressure further comprises determining one or more of: mouth breathing, nasal breathing, snorting, and/or apnea.

40. The method of claim 38, wherein determining the respiration pattern comprises determining physical activity.

41. The method of claim 37, further comprising monitoring a patient for a respiratory disorder based on the intraoral pressure.

42. The method of claim 37, further comprising determining if the intraoral pressure indicates if the subject is snoring or experiencing apnea.

43. The method of claim 37, further comprising determining, from the intraoral pressure, if the subject is experiencing bruxism.

44-46. (canceled)

47. The method of claim 37, further comprising generating the signal by deforming a deformable outer surface of the pressure sensor module while the dental appliance is worn by the subject to change the internal pressure within the airtight encapsulated cavity.

48. (canceled)

49. The method of claim 37, further comprising applying the dental appliance to the subject's mouth so that a pair of tooth-receiving portions fit onto the subject's teeth and the pressure sensor module is positioned adjacent to the subject's palate.

50-78. (canceled)

79. A dental appliance comprising:
a body forming one or more tooth-receiving cavities configured to be worn on a subject's teeth;
a sealed pressure sensor module coupled to the body, the sealed pressure sensor module including an airtight encapsulated cavity that is encapsulated by one or more fluid-resistant materials, wherein at least a portion of the one or more fluid-resistant materials forms a deformable surface, and a pressure sensor encapsulated within the airtight encapsulated cavity and configured to measure pressure within the airtight encapsulated cavity;
a power source;
a processor configured to receive pressure data from the pressure sensor and configured to process and store the pressure data; and
a circuitry configured to transmit information associated with the pressure data to an external device;
wherein the sealed pressure sensor module, power source, and circuitry are arranged along a curve of a lingual side the body that is configured to be positioned along a lingual portion of the subject's dental arch when the one or more tooth-receiving cavities are worn on the subject's teeth.

80. The dental appliance of claim 79, wherein the sealed pressure sensor module, power source, and circuitry are within a pocket formed on the lingual side of the body.

81. The dental appliance of claim 79, further comprising a temperature sensor that is located at an anterior position of the body.

82. The dental appliance of claim 79, where the sealed pressure sensor module, power source, and circuitry are arranged on a substrate including a serpentine and/or zig-zag pattern configured to flex.
